(19) United States
(12) Patent Application Publication
(10) Pub. No.: US 2023/0212571 A1
(43) Pub. Date: Jul. 6, 2023

(54) COMPOSITIONS AND METHODS FOR DELIVERING POLYNUCLEOTIDES

(71) Applicant: NeOnc Technologies, Inc., Los Angeles, CA (US)

(72) Inventor: Thomas C. CHEN, La Canada, CA (US)

(21) Appl. No.: 18/001,082

(22) PCT Filed: Jun. 8, 2021
(86) PCT No.: PCT/US2021/036310

§ 371 Date: Dec. 8, 2022

Related U.S. Application Data
(60) Provisional application No. 63/049,282, filed on Jul. 8, 2020.
(60) Provisional application No. 63/035,958, filed on Jun. 8, 2020.

(51) Int. Cl. C12N 15/113; A61P 7/00

(52) U.S. Cl. CPC C12N 15/113; A61P 7/00; C12N 2310/141

(57) ABSTRACT

The disclosure relates to compositions, methods, and kits for using perillyl alcohol and/or an Argonaute protein (e.g., Ago-2) to deliver a polynucleotide to a cell. The disclosure also relates to compositions, methods, and kits for treating a condition by using perillyl alcohol and/or an Argonaute protein (e.g., Ago-2) to deliver a polynucleotide to a cell. The conditions may be brain vascular diseases and brain tumors.

### CROSS REFERENCE TO RELATED APPLICATIONS

This application claims priority to U.S. Provisional Application No. 63/035,958 (filed on Jun. 8, 2020) and 63/049,282 (filed on Jul. 8, 2020), the disclosure of each of which is incorporated by reference herein in their entirety.

### FIELD OF THE INVENTION

The invention relates to using perillyl alcohol (POH) and/or an Argonaute protein to deliver a polynucleotide (e.g., a microRNA or miRNA) to a cell, particularly a brain endothelial cell. The invention also relates to compositions, methods, and kits for inhibiting angiogenesis and/or treating a condition by using perillyl alcohol (POH) and/or an Argonaute protein to deliver a polynucleotide (e.g., a miRNA) to an endothelial cell. The conditions include cerebrovascular disorders and brain tumors.

### BACKGROUND

MicroRNAs (miRNAs or miRs) are a class of regulatory RNAs that post-transcriptionally regulate gene expression. MiRNAs are evolutionarily conserved, small non-coding RNA molecules of approximately 18 to 25 nucleotides in length. Weiland et al., (2012) RNA Biol. 9(6):850-859. Bartel D P (2009) Cell 136(2):215-233. Each miRNA can downregulate hundreds of target mRNAs comprising partially complementary sequences to the miRNAs. MiRNAs act as repressors of target mRNAs by promoting their degradation, or by inhibiting translation. Braun et al. (2013) Adv. Exp. Med. Biol. 768:147-163.

MicroRNAs are promising targets for drug and biomarker development. Weiland et al. (2012) RNA Biol. 9(6):850-859. Target recognition requires base pairing of the miRNA 5′ end nucleotides (seed sequence) to complementary target mRNA regions located typically within the 3′UTR. Bartel D P (2009) Cell 136(2):215-233. Additionally, the recent detection of miRNPs (ribonucleoproteins), which contain associated miRNAs, in body fluids points towards their potential value as biomarkers for tissue injury. Laterza et al. (2009) Clin. Chem. 55:1977-1983; Ai et al. (2010) Biochem. Biophys. Res. Commun. 391:73-77. Additionally, it is also possible that miRNPs can act as paracrine and endocrine regulators of gene expression. Valadi et al. (2007) Nat. Cell Biol. 9:654-659; Williams et al. (2013) Proc. Natl. Acad. Sci. USA 110:4255-4260.

“RNA interference, or RNAi” is a form of post-transcriptional gene silencing (“PTGS”) and describes effects that result from the introduction of double-stranded RNA into cells (reviewed in Fire, A. Trends Genet 15:358-363 (1999); Sharp, P. Genes Dev 13:139-141 (1999); Hunter, C. Curr Biol 9: R440-R442 (1999); Baulcombe. D. Curr Biol 9: R599-R601 (1999); Vaucheret et al. Plant J 16: 651-659 (1998)). RNA interference, commonly referred to as RNAi, offers a way of specifically inactivating a cloned gene, and is a powerful tool for investigating gene function. The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. While RNAi was shown initially to work well in lower eukaryotes, for mammalian cells, it was thought that RNAi might be suitable only for studies on the oocyte and the preimplantation embryo. More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, “short interfering RNA” (siRNA, also referred to as small interfering RNA) were applied to cultured cells by transfection in oligofectamine micelles. These RNA duplexes were too short to elicit sequence-nonspecific responses like apoptosis, yet they efficiently initiated RNAi. Many laboratories then tested the use of siRNA to knock out target genes in mammalian cells. The results demonstrated that siRNA works quite well in most instances.

The systemic delivery of miRNA without transfection reagents (naked delivery) has been successfully accomplished for the reduction of tumor metastasis in the mouse liver, by showing that naked miRNA can be internalized by tumor cells. In addition, intravenous injection of naked miRNA was shown to also enter virally infected liver cells. These applications were focused solely on the systemic delivery of miRNA into highly vascularized liver as the target organ, and not relevant to the neurovasculature.

Intranasal delivery of a drug offers a novel non-invasive therapy to bypass the blood brain barrier and to rapidly deliver pharmaceutical agents to the CNS directly. Intranasally administered drugs reach the parenchymal tissues of the brain, spinal cord and/or cerebrospinal fluid (CSF) within minutes. In addition to delivery via the olfactory tract and trigeminal nerves, it appears from animal studies that the therapeutic drug is also delivered systemically through the nasal vasculature. Hashizume et al. New therapeutic approach for brain tumors: intranasal delivery of telomerase inhibitor GRN163. Neuro-oncology 10: 112-120, 2008. Thorne et al. Delivery of insulin-like growth factor-1 to the rat brain and spinal cord along olfactory and trigeminal pathways following intranasal administration. Neuroscience 127: 481-496, 2004. Intranasal delivery of therapeutic agents may provide a systemic method for treating other types of cancers, such as lung cancer, prostate cancer, breast cancer, hematopoietic cancer, and ovarian cancer, etc.

There is still a need to effectively deliver polynucleotides (e.g., miRNA, siRNA, oligonucleotides) to cells and in the treatment of various conditions.

### SUMMARY OF THE INVENTION

In a first embodiment, the present invention provides a method of delivering a polynucleotide to a cell, the method comprising: contacting the cell with the polynucleotide and perillyl alcohol (POH). Further embodiments exist, wherein the polynucleotide and POH are provided in one composition. Further embodiments exist, wherein the polynucleotide and POH are provided in separate compositions. Further embodiments exist, wherein the polynucleotide and POH are mixed prior to contacting the cell. Further embodiments exist, wherein the polynucleotide is a microRNA (miRNA). Further embodiments exist, wherein the miRNA is miR-18a. Further embodiments exist, wherein the polynucleotide is a short interfering RNA (siRNA) or a short-hairpin RNA (shRNA).

In a second embodiment, the present invention provides a method of delivering a polynucleotide to a cell, the method comprising: contacting the cell with the polynucleotide, perillyl alcohol (POH) and an Argonaute protein or a variant thereof. Further embodiments exist, wherein the Argonaute protein is Argonaute-2 (Ago-2). Further embodiments exist, wherein the polynucleotide, POH and the Argonaute protein or a variant thereof are provided in one composition. Further embodiments exist, wherein the polynucleotide, POH and the Argonaute protein or a variant thereof are provided in two or three compositions. Further embodiments exist, wherein the polynucleotide, POH and the Argonaute protein or a variant thereof are mixed prior to contacting the cell. Further embodiments exist, wherein the polynucleotide is a microRNA (miRNA). Further embodiments exist, wherein the miRNA is miR-18a. Further embodiments exist, wherein the polynucleotide is a short interfering RNA (siRNA) or a short-hairpin RNA (shRNA).

In a third embodiment, the present invention provides a method of delivering a polynucleotide to a subject, the method comprising administering the polynucleotide, perillyl alcohol (POH), and optionally an Argonaute protein or a variant thereof to the subject. Further embodiments exist, wherein the Argonaute protein is Argonaute-2 (Ago-2). Further embodiments exist, wherein the polynucleotide is a microRNA (miRNA). Further embodiments exist, wherein the polynucleotide is a short interfering RNA (siRNA) or a short-hairpin RNA (shRNA). Further embodiments exist, wherein the miRNA is miR-18a. Further embodiments exist, wherein the polynucleotide, POH and optionally the Argonaute protein or a variant thereof are provided in one composition. Further embodiments exist, wherein the polynucleotide, POH and optionally the Argonaute protein or a variant thereof are provided in two or three compositions. Further embodiments exist, wherein the polynucleotide, POH and optionally the Argonaute protein or a variant thereof are mixed prior to administration to the subject.

In a fourth embodiment, the present invention provides a method of treating a condition in a subject, the method comprising: administering a polynucleotide; perillyl alcohol (POH); and optionally an Argonaute protein or a variant thereof to the subject. Further embodiments exist, wherein the polynucleotide is a microRNA (miRNA). Further embodiments exist, wherein the miRNA is miR-18a. Further embodiments exist, wherein the polynucleotide is a short interfering RNA (siRNA) or a short-hairpin RNA (shRNA). Further embodiments exist, wherein the Argonaute protein is Argonaute-2 (Ago-2). Further embodiments exist, wherein the polynucleotide, POH and optionally the Argonaute protein or a variant thereof are provided in one composition. Further embodiments exist, wherein the polynucleotide, POH and optionally the Argonaute protein or a variant thereof are provided in separate compositions. Further embodiments exist, wherein the polynucleotide, POH and optionally the Argonaute protein or a variant thereof are mixed prior to administration to the subject. Further embodiments exist, wherein the condition is a neurovascular disease. Further embodiments exist, wherein the condition is stroke. Further embodiments exist, wherein the condition is a tumor. Further embodiments exist, wherein the condition is brain tumor, glioma, glioblastoma, and/or glioblastoma multiforme (GBM). Further embodiments exist, wherein the condition is spinal cord injury. The method of claim 24, wherein the administration is intranasal, intratumoral, intracranial, intraventricular, intrathecal, epidural, intradural, intravascular, intravenous, intraarterial, intramuscular, subcutaneous, intraperitoneal, or oral. Further embodiments exist, wherein the administration is given once, twice, three or more times. Further embodiments exist, wherein the method further comprises administering an anti-angiogenic drug to the subject. Further embodiments exist, wherein the method further comprises administering a chemotherapeutic agent to the subject.

In a fifth embodiment, the present invention provides a kit comprising: a polynucleotide; perillyl alcohol (POH); and instructions for delivering the polynucleotide to a cell or a subject using POH. Further embodiments exist, wherein the polynucleotide is a microRNA (miRNA). Further embodiments exist, wherein the miRNA is miR-18a. Further embodiments exist, wherein the polynucleotide is a short interfering RNA (siRNA) or a short-hairpin RNA (shRNA).

In a sixth embodiment, the present invention provides a kit comprising: a polynucleotide; perillyl alcohol (POH); an Argonaute protein or a variant thereof; and instructions for delivering the polynucleotide to a cell or a subject using the POH and the Argonaute protein or a variant thereof. Further embodiments exist, wherein the polynucleotide is a microRNA (miRNA). Further embodiments exist, wherein the miRNA is miR-18a. Further embodiments exist, wherein the polynucleotide is a short interfering RNA (siRNA) or a short-hairpin RNA (shRNA).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Exemplary embodiments are illustrated in referenced figures. It is intended that the embodiments and figures disclosed herein are to be considered illustrative rather than restrictive.

FIGS. 1A-1E. MiR-18a normalizes the levels of the pro-angiogenic factor PAI-1 in AVM-BEC. In all cases, patient-derived ECs underwent the indicated treatments under shear flow conditions (12 dyn/cm2) to reproduce arterial flow. FIG. 1A: Proteome array of 55 angiogenesis-related factors performed in lysates obtained from AVM-BECs treated with a scramble microRNA as the negative control (Scr miRNA, black bars) or with miR-18a (white bars). The levels of the factors whose expression was observed in the AVM-BECs, are shown as fold change relative to scramble microRNA-treated AVM-BECs (n=1, no statistics performed). FIG. 1B: Representative images of western blot analysis of bFGF, ET-1, PAI-1, and VEGF from the indicated cell lysates. GAPDH was used as a loading control. Ns, not significant; **, P<0.01; ***, P<0.001 (bFGF: P=0.1856, n=4; ET-1: P=0.3903, n=3, PAI-1: P<0.001, n=8; VEGF: P=0.0029, n=4. In all cases, scramble microRNA-treated vs miR-18a-treated AVM-BECs are compared using 1-way ANOVA followed by Bonferroni's multiple comparison test). FIG. 1C: ELISA analysis of PAI-1 and VEGF using supernatants from control BEC (black bars) or AVM-BEC (white bars) untreated (unt) or treated with scramble microRNA (Scr), miR-18a, thrombospondin 1 (TSP-1), or small interfering RNA against ID1 (siID1) (at least, n=3 per condition). Stock solution of TSP-1 recombinant protein was prepared in PBS and further diluted in culture medium to be administered to the cells. Results are expressed as fold-change relative to scramble microRNA-treated BEC. **, P<0.01 (In PAI-1 graph: P=0.0032, AVM Scr vs AVM miR-18a; P=0.0027, AVM Scr vs AVM TSP-1; P=0.0022, AVM Scr vs AVM siID1. In VEGF graph: P=0.0079, AVM Scr vs AVM miR-18a; P=0.6036, AVM Scr vs AVM TSP-1; P>0.9999, AVM Scr vs AVM siID1. In all cases, 1-way ANOVA followed by Bonferroni's multiple comparison test was used). Completely untreated BECs and AVM-BECs were included as controls for microRNA treatments. No significant differences were observed between the untreated cells and the corresponding scramble microRNA-treated cells (P>0.9999). Additionally, a small interfering control construct (siCTL) was used as a control of transfection using Lipofectamine for siID1 transfections. No significant differences were observed either between the untreated cells and the corresponding siCTL-treated cells (P>0.9999), or between the siCTL-transfected cells and the corresponding siID1 cells (P>0.9999), except in the case of PAI-1 in AVM-BECs, which showed statistical significance [^{##}, P<0.01 (P=0.0026, AVM siID1 vs AVM siCTL)]. FIG. 1D: Proteins from three patient-derived AVM-BEC samples were immunoprecipitated with TSP-1 antibody and analyzed for PAI-1. TSP-1 protein content in each sample was also determined. Negative control (C−) received the same concentration of TSP-1 antibody, but the coupling resin was replaced with control agarose resin that is not amine-reactive. FIG. 1E: ELISA analysis of VEGF using control BEC (black bars) or AVM-BEC (white bars) untransfected or transfected either with a small interfering control construct (siCTL) or a small interfering RNA against PAI-1 (siPAI-1), and untreated (unt) or treated with scramble microRNA (Scr) or miR-18a (at least n=5 per condition). Results are expressed as fold-change relative to untransfected BECs. Ns, not significant (P>0.9999, AVM siPAI-1 Scr vs AVM siPAI-1 miR-18a, 1-way ANOVA followed by Bonferroni's multiple comparison test); ***, P<0.001 (P=0.0007, AVM siCTL Scr vs AVM siCTL miR-18a, 1-way ANOVA followed by Bonferroni's multiple comparison test)). ^{###}, P<0.001 (P=0.0003, AVM siCTL unt vs AVM siPAI-1 unt, 1-way ANOVA followed by Bonferroni's multiple comparison test).

FIGS. 2A-2F. MiR-18a blocks BMP4 signaling in AVM-BEC. In all cases, patient-derived ECs underwent the indicated treatments under shear flow conditions (12 dyn/cm²) to reproduce arterial flow. FIG. 2A: Sequence alignment of miR-18a and BMP4 obtained from microRNA.org. FIG. 2B: ECs were transiently transfected with a BMP4 3′UTR luciferase reporter, and then left untreated or treated with scramble microRNA (Scr miR) or miR-18a. MiR-18a reduces the luciferase activity of the BMP4 3′UTR reporter in ECs. Results are expressed as fold change relative to untreated BMP4 3′UTR luciferase reporter transfected ECs. ***, P<0.001 (n=6, 1-way ANOVA followed by Bonferroni's multiple comparison test). FIG. 2C: RT-qPCR data of BMP4, ALK2, ALK1, ALK5, MGP and BMP9 expression. RNA was extracted from cell lysates of BEC and AVM-BEC treated with scramble microRNA (Scr) or miR-18a (18a). Results are expressed as fold-change relative to scramble microRNA-treated BEC. B2M was used for sample normalization. Ns, not significant; *, P<0.05; **, P<0.01; ****, P<0.0001 (BMP4: P>0.9999, BEC Scr vs BEC miR-18a; P=0.0022, AVM-BEC Scr vs AVM-BEC miR-18a; n=3. ALK2: P=0.1607, BEC Scr vs BEC miR-18a; P<0.0001, AVM-BEC Scr vs AVM-BEC miR-18a; n=3. ALK1: P=0.0482, BEC Scr vs BEC miR-18a; P=0.0013, AVM-BEC Scr vs AVM-BEC miR-18a; n=3. ALK5: P>0.9999, BEC Scr vs BEC miR-18a; P=0.0072, AVM-BEC Scr vs AVM-BEC miR-18a; n=3. MGP: P>0.9999, BEC Scr vs BEC miR-18a; P<0.0001, AVM-BEC Scr vs AVM-BEC miR-18a; n=6. BMP9: P=0.4360, BEC Scr vs BEC miR-18a; P=0.2011, AVM-BEC Scr vs AVM-BEC miR-18a; n=3. In all cases, 1-way ANOVA followed by Bonferroni's multiple comparison tests were used). FIG. 2D: Representative western blots of mature TGF-β, phospho Smad3 (P-Smad3), total Smad3 (T-Smad3), phospho Smad1/5 (P-Smad1/5), total Smad1/5/9 (T-Smad1/5/9), total Smad4 (T-Smad4), and GAPDH as the loading control, extracted from the from the indicated cell lysates. FIG. 2E: Bar graphs represent the data from western blot studies as fold change relative to scramble microRNA treated BECs. Ns, not significant; ****, P<0.0001 (TGF-β/GAPDH: P>0.9999, n=3; P-SMAD3/T-SMAD3: P>0.9999, n=3; P-SMAD1/5/T-SMAD1/5/9: P<0.0001, n=5; SMAD4/GAPDH: P>0.9999, n=5. In all cases, scramble microRNA-treated vs miR-18a-treated AVM-BECs are compared using 1-way ANOVA followed by Bonferroni's multiple comparison test). FIG. 2F: ELISA analysis of VEGF in supernatants from BECs (black bars, n=3 per condition) and AVM-BECs (white bars, n=4 per condition) transfected with ALK2 wild-type (ALK2^{WT}) or constitutively active ALK2 (ALK2^{Q207D}) and treated with scramble microRNA (Scr miR) or miR-18a. non-transfected cells served as basal level controls. Data from untreated transfected cells have been added as controls for transfections. No significant differences were observed between the corresponding untreated and scramble microRNA-treated cells (P>0.9999). Ns, not significant (in all cases P>0.9999, 1-way ANOVA followed by Bonferroni's multiple comparison test); **, P<0.01 (P=0.0030, 1-way ANOVA followed by Bonferroni's multiple comparison test).

FIGS. 3A-3C. MiR-18a blocks HIF-1α expression in AVM-BEC only when cultured under normoxia. In all cases, patient-derived ECs underwent the indicated treatments under shear flow conditions (12 dyn/cm²) to reproduce arterial flow. For hypoxic experiments, cells were cultured at 3% O₂. FIG. 3A: Sequence alignment of miR-18a and HIF-1α obtained from microRNA.org. FIG. 3B: qPCR data of HIF-1α expression in cell extracts of BECs and AVM-BECs treated with either scramble microRNA (Scr) or miR-18a (18a) in normoxic (left) and in hypoxic (right) conditions. Results are expressed as fold-change relative to scramble microRNA-treated BEC. B2M was used for sample normalization. Ns, not significant; **, P<0.01 (HIF-1α in normoxia: P>0.9999, BEC Scr vs BEC miR-18a; P=0.0025, AVM-BEC Scr vs AVM-BEC miR-18a; n=3. HIF-1α in hypoxia: P=0.4837, BEC Scr vs BEC miR-18a; P>0.9999, AVM-BEC Scr vs AVM-BEC miR-18a; n=3. In all cases, 1-way ANOVA followed by Bonferroni's multiple comparison tests were used). FIG. 3C: ELISA analysis of PAI-1 and VEGF in supernatants from BECs (black bars, n=3 per condition) and AVM-BECs (white bars, n=3 per condition) treated with scramble microRNA (Scr), miR-18a, thrombospondin 1 (TSP-1), or small interfering RNA against ID1 (siID1) in hypoxia. Stock solution of TSP-1 recombinant protein was prepared in PBS and further diluted in culture medium to be administered to the cells. Results are expressed as fold-change relative to scramble microRNA-treated BEC. *, P<0.05 (In PAI-1 graph: P=0.0378, AVM Scr vs AVM miR-18a; P=0.3917, AVM Scr vs AVM TSP-1; P=0.1701, AVM Scr vs AVM siID1. In VEGF graph: P>0.9999, AVM Scr vs AVM miR-18a; P>0.9999, AVM Scr vs AVM TSP-1; P>0.9999, AVM Scr vs AVM siID1. In all cases, 1-way ANOVA followed by Bonferroni's multiple comparison tests were used).

FIGS. 4A-4E. MiR-18a decreases AVM-BEC invasiveness. In all cases, patient-derived ECs underwent the indicated treatments under shear flow conditions (12 dyn/cm²) to reproduce arterial flow. FIG. 4A: Representative images of the invasion assays through a Matrigel-coated Boyden chamber, performed with BEC (upper panels) and AVM-BEC (lower panels) treated with scramble microRNA or miR-18a. Scale bar, 200 μm. After overnight treatments, the number of invaded cells per field was counted. Ns, not significant (P>0.9999); **, P<0.01 (P=0.0012) (n=3 per condition, 1-way ANOVA followed by Bonferroni's multiple comparison test). FIG. 4B: RT-qPCR data of MMP2 and MMP9 expression in cell extracts of BEC and AVM-BEC treated with either scramble microRNA (Scr) or miR-18a (18a). Results are expressed as fold-change relative to scramble microRNA-treated BEC. Ns, not significant; **, P<0.01; ***, P<0.001 (MMP2: P=0.9946, BEC Scr vs BEC miR-18a; P=0.0004, AVM-BEC Scr vs AVM-BEC miR-18a; n=3. MMP9: P>0.9999, BEC Scr vs BEC miR-18a; P=0.0091, AVM-BEC Scr vs AVM-BEC miR-18a; n=3. In all cases, 1-way ANOVA followed by Bonferroni's multiple comparison tests were used.). FIG. 4C: Western blot analysis of MMP2, MMP9, ADAM10, and GAPDH as loading control from the indicated cell lysates. Representative images of western blot. Bar graphs represent fold-change relative to scramble microRNA-treated BECs. Ns, not significant (P=0.9409); *, P<0.05 (P=0.0132); ***, P<0.001 (n=3. In all cases, 1-way ANOVA followed by Bonferroni's multiple comparison tests were used). FIG. 4D: Representative images of BEC and AVM-BEC stained for MMP2, MMP9 treated with scramble microRNA or miR-18a. Darker-shading denotes positive staining. Scale bars, 200 μm. FIG. 4E: Ratio of MMP2/MMP9 protein levels. Ns, not significant (P=0.4002) *, P<0.05 (P=0.0146); **, P<0.01 (P=0.0022) (n=3, 1-way ANOVA followed by Bonferroni's multiple comparison test).

FIGS. 5A-5C. Pharmacokinetic studies of miR-18a in serum and brain. MiR-18a (0.8 nmol/mouse of miR-18a) was administered intravenously (IV) or intranasally (IN) to C57BL/6 mice. Brains and serum were harvested immediately after administration (0 h), and after 0.5, 1, 2, 4, 24 and 48 h. Mice treated with scramble microRNA served as the background control. For positive controls, serum and brain tissue homogenates were spiked with the same amount of miR-18a. RT-qPCR analysis were performed to detect the levels of miR-18a in serum (FIG. 5A) or brains (FIG. 5B) at each time-point. FIG. 5C: Bar graphs represent fold change of miR-18a in mice treated IN with miR-18a with 0.3% NEO100 (white bars) or without 0.3% NEO100 (black bars). *, P<0.05; ****, P<0.0001 (Exact P values comparing miR-18a alone vs miR-18a+NEO10 for each time point in the serum, IN administration: 0 h, P=0.0851; 0.5 h, P=0.0148; 1 h, P<0.0001; 2 h, P>0.9999; 4 h, P=0.7449; 24 h, P=0.7305; 48 h, P>0.9999. Exact P values comparing miR-18a alone vs miR-18a+NEO10 for each time point in the brains, IN administration: 0 h, P<0.0001; 0.5 h, P>0.9999; 1 h, P=0.0746; 2 h, P=0.4369; 4 h, P>0.9999; 24 h, P>0.9999; 48 h, P>0.9999) (n=6 per time-point and condition, 2-way ANOVA followed by Bonferroni's multiple comparison test). In all cases, the average fold changes relative to the basal levels of miR-18a detected in the scramble microRNA-treated mice were represented as mean±SEM using bar graphs.

FIGS. 6A-6C. In vivo effects of miR-18a on brain AVMs. Mice co-treated IN with 0.08 nmol/mouse of miR-18a or scramble microRNA and Ago-2 for 2-weeks underwent in vivo ultra-high-resolution computed tomography angiography (CTA). Untreated Mgp^{+/+} (WT), Mgp^{+/-} and Mgp^{-/-} (AVM) were included as controls. In FIG. 6A and FIG. 6B, three distinct views from the in vivo CTA scans of the mouse cerebrum are exhibited: transverse, coronal, and sagittal views of the brain. To be noted in the transverse views of both FIG. 6A and FIG. 6B is the presence of thalamic penetrating vessels in the inferior/ventral (bottom) region of the image by arrows, and the cortical vessels in the superior/dorsal (top) region of the image by arrows. In the coronal views of FIG. 6A and FIG. 6B, the Circle of Willis (CoW) is prominently displayed in the top area of the image asterisks. The CoW is formed from the two internal carotid arteries (ICA), which are derived from the two anterior cerebral arteries (ACA); the basilar artery (BA) branches into the posterior (PCA) and superior (SCA) cerebral arteries, and two vertebral arteries (VA). In the sagittal views in FIG. 6A and FIG. 6B, both the azygous of the anterior cerebral artery (AzACA) and the basilar artery is indicated in the top right and bottom regions of the image by the arrows. FIG. 6A: Volume-rendered 3D images of cerebral vasculature in Mgp^{+/+} (WT), Mgp^{+/-} and Mgp^{-/-} (AVM) mice. Mgp^{-/-} shows poor circulation, lower vascular density, abnormal vessels and direct connections between arteries and veins characteristic of AVM niduses, when compared to heterozygous and WT mice. FIG. 6B: Volume-rendered 3D images of the cerebral vasculature in Mgp^{-/-} mice treated with scramble microRNA (left), miR-18a (middle) and miR-18a+0.3% NEO100 (right). Treatment with miR-18a improves circulation in cerebral vasculature of the Mgp^{-/-} mice, as evidenced by the lower distortion of the CoW and fewer direct shuntings of arteries and veins, indicative of AVM niduses. Arrows point at vascular features where miR-18a-induced improvements are observed, showing a mature development of middle cerebral arteries (MCA). MiR-18a-treated Mgp^{-/-} mice with and without co-administration of NEO100 appear to have a similar vasculature, suggesting that NEO100 does not interfere with the therapeutic effects of miR-18a. Black bars, 0.1 cm. The videos showing the volume-rendered 3D cerebral vasculature were included in Data. FIG. 6C: Quantitative analysis of in vivo CTA data showed significant improvement in brain vasculature in miR-18a-treated mice compared to untreated and scramble microRNA-treated (Scr) mice. The bar graphs represent the data from untreated (unt) (n=3), scramble microRNA-treated (Scr) (n=3) and miR-18a-treated [with (18a/NEO) (n=3) or without (18a) (n=3) 0.3% NEO100] Mgp^{-/-} (AVM) mice, as well as of untreated Mgp^{+/-} (n=5) and Mgp^{+/+} (WT) (n=5) mice. Vascular density (expressed as percentage, %) for regions of interest in CoW and AzACA and its branches were normalized with respect to wildtype mice. Each condition was tested for significance relative to untreated Mgp^{-/-} (AVM) mice, using 1-way ANOVA followed by Dunnett's multiple comparison test. Ns, not significant; *, P<0.05; **, P<0.01; ***, P<0.001; ****, P<0.0001 [P-values of CoW graph (vs unt Mgp^{-/-}): P>0.9999, Scr Mgp^{-/-}; P<0.0001, 18a Mgp^{-/-}; P=0.0002, 18a+NEO100 Mgp^{-/-}; P<0.0001, Mgp^{+/-}; P<0.0001, Mgp^{+/+}. P-values of AzACA graph (vs unt Mgp^{-/-}): P=0.5830, Scr Mgp^{-/-}; P=0.0237, 18a Mgp^{-/-}; P=0.0186, 18a+NEO100 Mgp^{-/-}; P=0.0146, Mgp^{+/-}; P=0.0033, Mgp^{+/+}]. No significant differences were observed between miR-18a-treated Mgp^{-/-} mice with or without NEO100 (CoW: P=0.4710; AzACA: P=8605; unpaired two-tailed t-test).

FIG. 7. MiR-18a blocks the expression of BMP4, PAI-1, VEGF, HIF-1α and the activation of the BMP4-downstream effectors Smad1/5 in the Mgp^{-/-} in vivo model of AVM. Representative images of brain sections from mice co-treated IN with 0.08 nmol/mouse of scramble microRNA (n=6), miR-18a (n=10) or miR-18a+0.3% NEO100 (n=3), and Ago-2 for 2-weeks. Untreated Mgp^{+/+} (WT, n=7), Mgp^{+/-} (n=8) and Mgp^{-/-} (AVM, n=7) were included as controls. Darker-shading (in some cases indicated with arrows for clarity purposes) denotes positive staining. The expression of BMP4, PAI-1, VEGF, and HIF-1α, as well as the activation (phosphorylation) of Smad1/5 was higher in brains from untreated and scramble microRNA-treated Mgp^{-/-} (AVM) mice, compared to Mgp^{+/+} (WT) and/or Mgp^{+/-} mice. Treatment with miR-18a, with or without NEO100, decreased the expression of BMP4, PAI-1, VEGF, and HIF-1α, as well as the activation of the BMP4-downstream effectors Smad1/5. No differences were observed in the phosphorylation of the TGF-β-downstream effector Smad3 amongst the different conditions. White scale bars, 50 μm; black scale bars, 100 μm.

FIGS. 8A-8C. MiR-18a restores the functionality of the bone marrow, lungs, spleens, and livers. Mice were co-treated IN with 0.08 nmol/mouse of miR-18a (n=10) or scramble microRNA (n=6) and Ago-2 for 2-weeks. Then, their organs were harvested in formalin and stained with hematoxylin & eosin. Untreated Mgp^{+/+} (WT, n=7), Mgp^{+/-} (n=8) and Mgp^{-/-} (AVM, n=7) were included as controls. FIG. 8A: Representative macroscopic pictures of the spleens of untreated Mgp^{+/+} (WT), untreated Mgp^{+/-} and of Mgp^{-/-} (AVM) mice with the following treatments: untreated (untr), scramble microRNA (Scr) and miR-18a (18a). FIG. 8B: Representative microscopic images of the bone marrow, lungs, spleens, livers, and kidneys. The bone marrows of scramble microRNA-treated Mgp^{-/-} mice showed high levels of adipose cells (asterisks) and very few, if any, megakaryocytes (arrows), a phenotype that was normalized by miR-18a treatment. The lungs of the scramble microRNA-treated Mgp^{-/-} had macrophages in their alveoli (arrows), indicating lung inflammation, not present with miR-18a treatment. The spleens of the scramble microRNA-treated Mgp^{-/-} mice showed absence of white matter, as evidenced by the lack of germinal center structures. The livers of the scramble microRNA-treated Mgp^{-/-} mice lacked structured hepatic lobules/bile ducts and showed blood vessels with abnormally thick walls (arrows) surrounded by stained cells indicative of inflammatory cell infiltration. No significant differences were observed in the kidneys from different conditions. Black scale bars, 50 μm. White scale bars, 200 μm. FIG. 8C: Bar graphs represent the levels of red blood cells (RBC), hemoglobin (HGB) and hematocrit (HCT) in the Mgp^{+/+} (WT), Mgp^{+/-}, scramble microRNA-treated Mgp^{-/-} (Scr) and miR-18a-treated Mgp^{-/-} mice. Each condition (n=3) was tested for significance relative to untreated Mgp^{+/+} (WT) mice, using 1-way ANOVA followed by Dunnett's multiple comparison test. Only the data from scramble microRNA-treated mice were found significantly different from WT mice. *, P<0.05; **, P<0.01 (relative to WT, exact P-values: P=0.0240, RBC; P=0.0086, HGB; P=0.0351, HCT).

FIGS. 9A-9C. Ago-2 increases the uptake of miR-18a to the nuclei of the AVM-BEC. Biotinylated hsa-miR-18a-5p sense and hsa-miR-18a-3p sense probes (FIGS. 9A-9B) or a biotinylated probe containing the scramble sequence of the miR-18a (FIG. 9C) were administered to AVM-BEC, previously transfected with a small interfering RNA against Ago-2 (siRNA Ago2) or small interfering control, as indicated in the figure. In some cases, the biotinylated microRNA probes were co-administered with Ago2 as the microRNA carrier and stabilizer. To detect the presence of the probes, cells were incubated with Alexa Fluor 488 streptavidin (lighter colored areas). DAPI was used to detect cell nuclei. FIGS. 9A-9B: Administration of miR-18a alone is enough to obtain AVM-BEC uptake without the need of any transfection reagent. Co-administration of miR-18a and Ago-2 as a microRNA carrier and stabilizer (Braksick S A, Fugate J E. Management of brain arteriovenous malformations. Curr Treat Options Neurol. 2015; 17(7):32), increases the levels of miR-18a detected inside the AVM-BEC, and specifically, inside their nuclei, as evidenced by the higher colocalization with DAPI. The nuclear localization is more evident with the miR-18a-5p (used for the studies presented in this manuscript) than with the miR-18a-3p biotinylated probes. Downregulation of the Ago-2 expression using a siRNA (Thermo Fisher Scientific) decreased the levels of miR-18a detected inside the cells, suggesting a role of the intracellular Ago-2 regardless of the presence of exogenous Ago-2. Colocalization data was quantified using Zeiss LSM 510 confocal microscope software and ImageJ and normalized to the data obtained with each corresponding miR-18a biotinylated probe administered alone. Statistical analyses were performed using GraphPad Prism 8. P<0.05 was considered statistically significant, using 1-way ANOVA followed by Bonferroni's multiple comparison tests (n=4 per condition). Ns, not significant; *, P<0.05; **, P<0.01 (5p probe graph: *, P=0.0279, **, P=0.0045. 3p probe graph: ns, P=0.2259; *, P=0.0292). FIG. 9C: Representative images of untreated cells and cells treated with a scramble biotinylated microRNA probe. No green fluorescence was observed in any of them, suggesting that the mechanism of miR-18a internalization into the AVM-BECs is sequence-specific. Scale bars, 100 μm.

FIG. 10. Levels of bFGF, ET-1, PAI-1 and VEGF are not altered in BECs by treatment with either scramble microRNA or miR-18a. Representative images of western blot analysis of bFGF, ET-1, PAI-1, and VEGF. GAPDH was used as a loading control. Statistical analyses were performed using GraphPad Prism 8. P<0.05 was considered statistically significant, using 1-way ANOVA, followed by Bonferroni's multiple comparison test (n=3). No significant differences were observed between any of the conditions.

FIGS. 11A-11B. Controls of co-immunoprecipitation. FIG. 11A: Proteins from three patient-derived BEC samples were immunoprecipitated with TSP-1 antibody and analyzed for PAI-1. TSP-1 protein content in each sample was also determined. Negative control (C−) received the same concentration of TSP-1 antibody, but the coupling resin was replaced with control agarose resin that is not amine-reactive. FIG. 11B: Controls of reverse co-immunoprecipitation were also performed. Proteins from patient-derived AVM-BEC (left, n=3) and BEC (right, n=3) samples were immunoprecipitated with PAI-1 antibody and analyzed for TSP-1. PAI-1 protein content in each sample was also determined. Negative control (C−) received the same concentration of PAI-1 antibody, but the coupling resin was replaced with control agarose resin that is not amine-reactive.

FIG. 12. MiR-18a does not alter the levels of secreted TGF-β. ELISA analysis of secreted TGF-β content in BEC (black bars) and AVM-BEC (white bars) supernatants, after treatment with scramble microRNA (Scr) or miR-18a (18a). The bar graph represents the fold change difference, relative to scramble microRNA treated BEC. Ns, not significant (P>0.9999, n=3, 1-way ANOVA followed by Bonferroni's multiple comparison test).

FIGS. 13A-13B. MiR-18a causes a general downregulation of the Notch signaling. A Notch signaling pathway PCR array was performed in AVM-BEC treated with scramble microRNA (grey bars) or with miR-18a (black bars). The graphs show the genes that were at least 2-fold downregulated (FIG. 13A) or upregulated (FIG. 13B) by miR-18a treatment. MiR-18a directly downregulated NOTCH1/2, the genes involved in Notch receptor processing ADAM10, ADAM17, NCSTN and PSEN1, as well as the transcriptional coactivators of Notch, MAML1/2, and their recruiter SNW1. Correspondingly, the Notch target genes CDKN1A, CFLAR, NFKB1, NFKB2, NRARP, CCND1, ERBB2, DTX1, PPARG, HES1/4, HEY2, FOS, FOSL1, CD44, and CHUK showed lower mRNA expression with miR-18a treatment. The Notch ligands DLL1/3/4 and JAG2 were upregulated in response to miR-18a treatment, suggesting a compensatory mechanism of AVM-BEC. MiR-18a also caused a significant decrease in the expression levels of AXIN1, CTNNB1, NOTCH2, HDAC1, EP300, and PSEN1, all related to apoptosis. The Notch target genes related to apoptosis CDKN1A, CFLAR, FOSL1, ID1, NFKB were downregulated with the treatment, but the Notch target genes related to apoptosis IL2RA and PTCRA were upregulated by miR-18a.

FIGS. 14A-14B. MiR-18a does not increase cell death in AVM-BEC. FIG. 14A: Apoptosis (n=7 per condition) and necrosis (n=4 per condition) levels in BEC and AVM-BEC after treatment with scramble microRNA (Scr) or miR-18a (18a), measured using the Cell Death Detection ELISA^{PLUS} Kit. FIG. 14B: Effects of miR-18a on apoptosis in BEC and AVM-BEC were determined using Propidium Iodide (PI)/Annexin V-488 staining by flow cytometry (n=3 per condition). Bar graph represents the percentage of apoptotic cells in BEC and AVM-BEC treated with scramble microRNA (Scr) or miR-18a (18a). In all cases, statistical analyses were performed using GraphPad Prism 8. P<0.05 was considered statistically significant, using 1-way ANOVA followed by Bonferroni's multiple comparison tests. Ns, not significant.

FIGS. 15A-15B. Treatment with miR-18a reduces the signs of fresh hemorrhage and the presence of abnormally enlarged blood vessels in the brain in the AVM Mgp^{-/-} mouse model. FIG. 15A: Signs of fresh hemorrhage were found in the scramble-treated Mgp^{-/-} mice, but not in the miR-18a-treated Mgp^{-/-}, in the Mgp+/− or in the Mgp+/+ mice. FIG. 15B: Representative pictures of the brains of the mice, and the corresponding CD-31 (lighter-colored areas) immunostaining analysis. DAPI was used to stain all cell nuclei within the brains. Immunostaining of the brains for CD31 (endothelial marker) showed a lack of normal vasculature in the scramble microRNA-treated Mgp^{-/-} mice, where the only blood vessels detected were abnormally enlarged and tortuous, with very low presence of normal-sized blood vessels, results consistent with the literature⁴. MiR-18a improved the appearance of the brain vasculature. Scale bars, 100 μm.

FIG. 16. Comparison in leakage between the scramble microRNA and the miR-18a-treated Mgp^{-/-} mice. Mice co-treated IN with 0.08 nmol/mouse of miR-18a or scramble microRNA and Ago-2 for 2-weeks underwent in vivo ultra-high-resolution computed tomography angiography (CTA). (A) In vivo CTA scans showing differences in vascular leakage between the scramble microRNA and the miR-18a-treated Mgp^{-/-} (AVM) mice. Focal spots of highly dense signal suggesting vascular leakage are indicated with asterisks.

FIGS. 17A-17B. MiR-18a shows no effect on calcifications found in the carotids of Mgp^{-/-} mice. Mice were co-treated IN with scramble microRNA or miR-18a with or without 0.3% NEO100 for 2-weeks, and then imaged using ultra high-resolution computed tomography angiography (CTA) (n=18). Untreated Mgp^{+/+} (WT), Mgp^{+/-} and Mgp^{-/-} (AVM) were included as controls. FIG. 17A: Representative two-dimensional sagittal images of the brains of untreated Mgp^{+/+} (WT), Mgp^{+/-} and Mgp^{-/-} (AVM) mice showing bright ‘surface’ spots on blood vessels indicative of calcifications in carotid arteries (arrows) of all Mgp^{-/-} (AVM). In comparison, Mgp^{+/+} (WT) and Mgp^{+/-} mice do not demonstrate similar findings. FIG. 17B: Treatment with scramble microRNA (left), miR-18a (middle) or MiR-18a+NEO100 (right) did not affect the presence or levels of calcification found in the carotid arteries of Mgp^{-/-} (AVM) mice.

FIGS. 18A-18B. Representative images of the bone marrow, lungs, spleens, livers, and kidneys of untreated Mgp^{-/-} mice (FIG. 18A) and 0.3% NEO100+miR-18a-treated Mgp^{-/-} mice (FIG. 18B). Black scale bars, 50 μm. White scale bars, 200 μm.

FIGS. 19A-19B. Validation of ID1 and PAI-1 knockdown. mRNA levels after transient transfection of ECs with a siRNA selectively targeting (FIG. 19A) ID1 (siID1) versus a nontargeted siRNA (siCTL); and (FIG. 19B) PAI-1 (siPAI-1) versus a nontargeted siRNA (siCTL).

### DETAILED DESCRIPTION OF THE INVENTION

The present disclosure provides for a method of delivering a polynucleotide to a cell. The method may comprise contacting the cell with the polynucleotide, perillyl alcohol (POH), and optionally an Argonaute protein or a variant thereof.

The polynucleotide, POH and optionally the Argonaute protein or a variant thereof may be provided in one composition or provided in two or three compositions. For example, the polynucleotide, POH and optionally the Argonaute protein or a variant thereof may be mixed prior to contacting the cell.

Also encompassed by the present disclosure is a method of delivering a polynucleotide to a subject. The method may comprise administering the polynucleotide, perillyl alcohol (POH), and optionally an Argonaute protein or a variant thereof to the subject.

The present disclosure provides for a method of treating a condition in a subject, the method comprising administering a polynucleotide, perillyl alcohol (POH), and optionally an Argonaute protein or a variant thereof to the subject.

The polynucleotide, POH and optionally the Argonaute protein or a variant thereof may be provided in one composition or provided in two or three compositions. For example, the polynucleotide, POH and optionally the Argonaute protein or a variant thereof may be mixed prior to administration to the subject.

The Argonaute protein may be Argonaute-2 (Ago-2).

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Examples of polynucleotides include, but are not limited to, coding or non-coding regions of a gene or gene fragment, exons, introns, messenger RNA (mRNA), transfer RNA, ribosomal RNA, short interfering RNA (siRNA), short-hairpin RNA (shRNA), micro-RNA (miRNA), antisense oligonucleotides, ribozymes, cDNA, recombinant polynucleotides, branched polynucleotides, plasmids, vectors, isolated DNA of any sequence, isolated RNA of any sequence, nucleic acid probes, and primers. One or more nucleotides within a polynucleotide can further be modified. The sequence of nucleotides may be interrupted by non-nucleotide components. A polynucleotide may also be modified after polymerization, such as by conjugation with a labeling agent.

SiRNAs may have 16-30 nucleotides, e.g., 16, 17, 18, 19, 20, 21, 22, 23, 24, 25, 26, 27, 28, 29, or 30 nucleotides. The siRNAs may have fewer than 16 or more than 30 nucleotides. The polynucleotides may include both unmodified siRNAs and modified siRNAs such as siRNA derivatives etc.

Cerebral Arteriovenous Malformations (AVMs) are brain vascular lesions comprising an abnormal tangle of vessels (nidus), in which arteries and veins are directly connected without an intervening capillary system. AVM affect approximately 300,000 people in the USA and can lead to serious neurological symptoms or death. Current medical treatments are highly invasive and can pose significant risks to nearby brain structures that regulate speech, movement, and sensory processing, highlighting the importance of developing more efficacious and safer therapies.

Human AVM-derived brain endothelial cells (AVM-BEC) have distinct and abnormal characteristics compared to normal BEC. Namely, AVM-BEC proliferate more rapidly, migrate faster, and produce aberrant vessel-like structures as compared to normal vasculature. AVM-BEC also express low levels of a key regulator of angiogenesis, thrombospondin-1 (TSP-1). These abnormal features are ameliorated with microRNA-18a (miR-18a) treatment. MiRNAs are small non-coding RNAs that inhibit gene expression by inducing cleavage or translational repression of messenger RNA (mRNA). Specifically, miR-18a inhibited TSP-1 transcriptional repressor, Inhibitor of DNA-binding protein-1 (Id-1), leading to increased TSP-1 levels and decreased vascular endothelial growth factor (VEGF)-A and VEGF-D secretion. miR-18a also regulated cell proliferation and improved tubule formation efficiency. Importantly, these effects were obtained with miRNA alone (naked delivery), in the absence of traditional transfection reagents, like lipofectamine, which cannot be used in vivo due to induced toxicity. Naked miRNAs have been shown to form complexes with circulating RNA-binding proteins, such as Argonaute-2 (Ago-2), a member of the Argonaute protein family, which also includes Ago-1, Ago-3, and Ago-4. In human cells, Ago-2 takes part in the RNA-induced silencing complex (RISC) to promote endonucleolytic cleavage of mRNA.

We show that AVM-BEC releases Ago-2, which can be used to enhance the entry of extracellular miR-18a into brain endothelial cells. In vitro studies show that Ago-2 in combination with miR-18a is functional and able to stimulate TSP-1 production. Furthermore, miR-18a in combination with Ago-2 can be delivered in vivo by intravenous administration, resulting in increased circulating serum TSP-1 and decreased VEGF-A. Thus Ago-2 may be used to decrease angiogenic activity in brain endothelial cells, making Ago-2 a biocompatible miRNA-delivery platform suitable for treating neurovascular diseases and brain tumors.

Various embodiments of the present invention provide a method of delivering a polynucleotide to a cell. The method may comprise or consist of providing the polynucleotide, POH and optionally an Argonaute protein (e.g., Ago-2) or a variant thereof; and contacting the cell with the polynucleotide, POH and optionally an Argonaute protein (e.g., Ago-2) or the variant thereof, thereby delivering the polynucleotide to the cell. In some embodiments, the polynucleotide, POH and optionally an Argonaute protein (e.g., Ago-2) or the variant thereof are provided in one composition. In other embodiments, the polynucleotide, POH and optionally an Argonaute protein (e.g., Ago-2) or the variant thereof are provided in separate compositions. Various embodiments of the present invention provide a kit for delivering a polynucleotide to a cell. The kit may comprise, or consist of, a quantity of a polynucleotide; a quantity of POH; and optionally a quantity of an Argonaute protein (e.g., Ago-2) a variant thereof; and instructions for using the POH and optionally an Argonaute protein (e.g., Ago-2) or the variant thereof to deliver the polynucleotide.

Various embodiments of the present invention provide a method of delivering a polynucleotide to a cell. The method may comprise or may consist of providing the polynucleotide, POH and optionally an Argonaute protein (e.g., Ago-2) or a variant thereof; mixing the polynucleotide with the POH and optionally an Argonaute protein (e.g., Ago-2) or the variant thereof; and contacting the cell with the mixture of the polynucleotide, POH and optionally an Argonaute protein (e.g., Ago-2) or the variant thereof, thereby delivering the polynucleotide to the cell. In various embodiments, the polynucleotide and the Ago-2 or the variant thereof form a ribonucleoprotein complex in the mixture.

Various embodiments of the present invention provide a method of inhibiting or suppressing angiogenesis in a subject. The method may comprise or may consist of providing a polynucleotide, POH and optionally an Argonaute protein (e.g., Ago-2) or a variant thereof, administering a therapeutically effective amount of the polynucleotide, POH and optionally an Argonaute protein (e.g., Ago-2) or the variant thereof to the subject, thereby inhibiting or suppressing angiogenesis in the subject. In some embodiments, the polynucleotide, POH and optionally an Argonaute protein (e.g., Ago-2) or the variant thereof are provided in one composition. In other embodiments, the polynucleotide, POH and optionally an Argonaute protein (e.g., Ago-2) or the variant thereof are provided in separate compositions. Various embodiments of the present invention provide a kit for inhibiting or suppressing angiogenesis. The kit may comprise or may consist of a quantity of a polynucleotide; a quantity of POH; and optionally an Argonaute protein (e.g., Ago-2) or a variant thereof; and instructions for using the polynucleotide, POH and optionally an Argonaute protein (e.g., Ago-2) or the variant thereof to inhibit or suppress angiogenesis. In some embodiment, the polynucleotide is capable of inhibiting or suppressing angiogenesis.

Various embodiments of the present invention provide a method of inhibiting or suppressing angiogenesis in a subject. The method may comprise or may consist of providing a polynucleotide, POH and optionally an Argonaute protein (e.g., Ago-2) or a variant thereof; mixing the polynucleotide with the POH and optionally an Argonaute protein (e.g., Ago-2) or the variant thereof; and administering a therapeutically effective amount of the mixture to the subject, thereby inhibiting, or suppressing angiogenesis in the subject. In various embodiments, the polynucleotide and the Ago-2 or the variant thereof form a ribonucleoprotein complex in the mixture. In some embodiment, the polynucleotide is capable of inhibiting or suppressing angiogenesis.

Various embodiments of the present invention provide a method of promoting angiogenesis in a subject. The method may comprise or may consist of providing a polynucleotide, POH and optionally an Argonaute protein (e.g., Ago-2) or a variant thereof, administering a therapeutically effective amount of the polynucleotide, POH and optionally an Argonaute protein (e.g., Ago-2) or the variant thereof to the subject, thereby promoting angiogenesis in the subject. In some embodiments, the polynucleotide, POH and optionally an Argonaute protein (e.g., Ago-2) or the variant thereof are provided in one composition. In other embodiments, the polynucleotide, POH and optionally an Argonaute protein (e.g., Ago-2) or the variant thereof are provided in separate compositions. Various embodiments of the present invention provide a kit for promoting angiogenesis. The kit may comprise or may consist of a quantity of a polynucleotide; a quantity of POH and optionally an Argonaute protein (e.g., Ago-2) or a variant thereof; and instructions for using the polynucleotide, POH and optionally an Argonaute protein (e.g., Ago-2) or the variant thereof to promote angiogenesis. In some embodiments, the polynucleotide can promote angiogenesis.

Various embodiments of the present invention provide a method of promoting angiogenesis in a subject. The method may comprise or may consist of providing a polynucleotide, POH and optionally an Argonaute protein (e.g., Ago-2) or a variant thereof; mixing the polynucleotide with POH and optionally an Argonaute protein (e.g., Ago-2) or the variant thereof; and administering a therapeutically effective amount of the mixture to the subject, thereby promoting angiogenesis in the subject. In various embodiments, the polynucleotide and the Ago-2 or the variant thereof form a ribonucleoprotein complex in the mixture. In some embodiments, the polynucleotide can promote angiogenesis.

Various embodiments of the present invention provide a method of treating, preventing, reducing the likelihood of having, reducing the severity of and/or slowing the progression of a condition in a subject. The method may comprise or may consist of providing a polynucleotide, POH and optionally an Argonaute protein (e.g., Ago-2) or a variant thereof, administering a therapeutically effective amount of the polynucleotide and POH and optionally an Argonaute protein (e.g., Ago-2) or the variant thereof to the subject, thereby of treating, preventing, reducing the likelihood of having, reducing the severity of and/or slowing the progression of a condition in the subject. In some embodiments, the polynucleotide and the POH and optionally an Argonaute protein (e.g., Ago-2) or the variant thereof are provided in one composition. In other embodiments, the polynucleotide and POH and optionally an Argonaute protein (e.g., Ago-2) or the variant thereof are provided in separate compositions. Various embodiments of the present invention provide a kit for treating, preventing, reducing the likelihood of having, reducing the severity of and/or slowing the progression of a condition. The kit may comprise or may consist of a quantity of a polynucleotide; a quantity of POH and optionally an Argonaute protein (e.g., Ago-2) or a variant thereof; and instructions for using the polynucleotide and POH and optionally an Argonaute protein (e.g., Ago-2) or the variant thereof to treat, prevent, reduce the likelihood of having, reduce the severity of and/or slow the progression of the condition in the subject.

Various embodiments of the present invention provide a method of treating, preventing, reducing the likelihood of having, reducing the severity of and/or slowing the progression of a condition in a subject. The method may comprise or may consist of: providing a polynucleotide and POH and optionally an Argonaute protein (e.g., Ago-2) or a variant thereof; mixing the polynucleotide and POH and optionally an Argonaute protein (e.g., Ago-2) or the variant thereof; and administering a therapeutically effective amount of the mixture to the subject, thereby treating, preventing, reducing the likelihood of having, reducing the severity of and/or slowing the progression of the condition in the subject. In various embodiments, the polynucleotide and the Ago-2 or the variant thereof form a ribonucleoprotein complex in the mixture.

In one embodiment, the polynucleotide, POH and optionally an Argonaute protein (e.g., Ago-2) or the variant thereof may be provided in one composition. In another embodiment, the polynucleotide, POH and optionally an Argonaute protein (e.g., Ago-2) or the variant thereof may be provided in two or three separate compositions. In various embodiments, the polynucleotide is administered at about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 nmol/L. In various embodiments, the polynucleotide is administered intratumorally, intracranially, intraventricularly, intrathecally, epidurally, intradurally, intravascularly, intravenously, intraarterially, intramuscularly, subcutaneously, intraperitoneally, intranasally, or orally. In various embodiments, the polynucleotide is administered once, twice, three or more times. In various embodiments, the mixture is administered 1-3 times per day, 1-7 times per week, or 1-9 times per month. In various embodiments, the polynucleotide is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. In various embodiments, the Argonaute protein (e.g., Ago-2) or the variant thereof is administered at about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 nmol/L. In various embodiments, the Argonaute protein (e.g., Ago-2) or the variant thereof is administered intratumorally, intracranially, intraventricularly, intrathecally, epidurally, intradurally, intravascularly, intravenously, intraarterially, intramuscularly, subcutaneously, intraperitoneally, intranasally, or orally. In various embodiments, the Argonaute protein (e.g., Ago-2) or the variant thereof is administered once, twice, three or more times. In various embodiments, the Argonaute protein (e.g., Ago-2) or the variant thereof is administered 1-3 times per day, 1-7 times per week, or 1-9 times per month. In various embodiments, the Argonaute protein (e.g., Ago-2) or the variant thereof is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years.

Various methods described herein may further comprise providing and administering a therapeutically effective amount of an anti-angiogenic drug to the subject. Various kits described herein may further comprise a quantity of an anti-angiogenic drug. Various methods described herein may further comprise providing and administering a therapeutically effective amount of a chemotherapeutic agent to the subject. Various kits described herein may further comprise a quantity of a chemotherapeutic agent.

Various embodiments of the present invention provide a composition. The composition may comprise or may consist of a polynucleotide, POH and optionally an Argonaute protein (e.g., Ago-2) or a variant thereof. In accordance with the present invention, the composition may be used for delivering the polynucleotide to a cell, inhibiting angiogenesis, promoting angiogenesis, and/or treating, preventing, reducing the likelihood of having, reducing the severity of and/or slowing the progression of a condition in a subject.

Various embodiments of the present invention provide a composition. The composition may comprise or may consist of a ribonucleoprotein complex of a polynucleotide and an Argonaute protein (e.g., Ago-2) or a variant thereof. In accordance with the present invention, the composition may be used for delivering the polynucleotide to a cell, inhibiting angiogenesis, promoting angiogenesis and/or treating, preventing, reducing the likelihood of having, reducing the severity of and/or slowing the progression of a condition in a subject.

In various embodiments, the subject is a human.

In various embodiments, the polynucleotide is a miRNA. The miRNA may be miR-18a or miR-128a. In some embodiment, the miRNA is capable of inhibiting or suppressing angiogenesis (e.g., miR-92, miR-92a, miR-221/22). In other embodiment, the miRNA can promote angiogenesis (e.g., miR-296, miR-126, mir-210, miR-130).

Various compositions described herein may be formulated for intratumoral, intracranial, intraventricular, intrathecal, epidural, intradural, intravascular, intravenous, intraarterial, intramuscular, subcutaneous, intraperitoneal, intranasal, or oral administration. Various compositions described herein may further comprise an anti-angiogenic drug. Various compositions described herein may further comprise a chemotherapeutic agent. Various compositions described herein may further comprise a pharmaceutically acceptable excipient. Various compositions described herein may further comprise a pharmaceutically acceptable carrier.

In accordance with the present invention, examples of anti-angiogenic drugs include but are not limited to Genentech/Roche (Bevacizumab/Avastin®), Bayer and Onyx Pharmaceuticals (sorafenib/Nexavar®), Pfizer (sutinib/Sutent®), GlaxoSmithKline (pazopanib/Votrient®), Novartis (everolimus/Affinitor®), Celgene (pomalidomide/Pomalyst®) and Ipsen and Active Biotech (tasquinimod/ABR-215050, CID 54682876).

In accordance with the present invention, examples of the chemotherapeutic agent include but are not limited to Temozolomide, Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Ipilimumab, Irinotecan, Mechlorethamine, Melphalan, Mercaptopurine, Methotrexate, Mitoxantrone, Ocrelizumab, Ofatumumab, Oxaliplatin, Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, ixabepilone, Estramustine, prednisone, methylprednisolone, dexamethasone or a combination thereof.

Various compositions, methods and kits of the present invention find utility in the treatment of various conditions, including but not limited to neurovascular disease, brain vascular disease, cerebra arteriovenous malformation (AMV), stroke, tumor or cancer, brain tumor, glioma, glioblastoma, and glioblastoma multiform (GBM).

A specific example of a monoterpene that may be used in the present invention is perillyl alcohol (commonly abbreviated as POH). Perillyl alcohol may be (S)-perillyl alcohol, (R)-perillyl alcohol, or a mixture of (S)-perillyl alcohol and (R)-perillyl alcohol.

Conditions to be treated by the present compositions and methods may include nervous system cancers, such as a malignant glioma (e.g., astrocytoma, anaplastic astrocytoma, glioblastoma multiforme), retinoblastoma, pilocytic astrocytomas (grade I), meningiomas, metastatic brain tumors, neuroblastoma, pituitary adenomas, skull base meningiomas, and skull base cancer. As used herein, the term “nervous system tumors” refers to a condition in which a subject has a malignant proliferation of nervous system cells.

Cancers that can be treated by the present compositions and methods include, but are not limited to, lung cancer, ear, nose and throat cancer, leukemia, colon cancer, melanoma, pancreatic cancer, mammary cancer, prostate cancer, breast cancer, hematopoietic cancer, ovarian cancer, basal cell carcinoma, biliary tract cancer; bladder cancer; bone cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia including acute myeloid leukemia, acute lymphoid leukemia, chronic myeloid leukemia, chronic lymphoid leukemia; liver cancer; lymphoma including Hodgkin's and Non-Hodgkin's lymphoma; myeloma; fibroma, neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas. U.S. Pat. No. 7,601,355.

The present compositions and methods may also be used to treat CNS disorders, including, without limitation, primary degenerative neurological disorders such as Alzheimer's, Parkinson's, psychological disorders, psychosis, and depression. Treatment may consist of the use of purified monoterpenes or sesquiterpenes alone or in combination with current medications used in the treatment of Parkinson's, Alzheimer's, or psychological disorders. For example, purified monoterpenes or sesquiterpenes may be used as a solvent for the inhalation of current medications used in the treatment of Parkinson's, Alzheimer's, or psychological disorders.

The present compositions and methods may be used to increase paracellular permeability, for example, paracellular permeability of endothelial cells or epithelial cells. The present compositions and methods may be used to increase blood brain barrier permeability.

The present compositions and methods may be used to decrease or inhibit angiogenesis. The present compositions and methods may decrease or inhibit production of pro-angiogenic cytokines, including, but not limited to, vascular endothelial growth factor (VEGF) and interleukin 8 (IL8).

The present compositions may be used in combination with angiogenesis inhibitors. Examples of angiogenesis inhibitors include, but are not limited to, angiostatin, angiozyme, antithrombin III, AG3340, VEGF inhibitors (e.g., anti-VEGF antibody), batimastat, bevacizumab (avastin), BMS-275291, CM, 2C3, HuMV833 Canstatin, Captopril, carboxyamidotriazole, cartilage derived inhibitor (CDI), CC-5013, 6-O-(chloroacetyl-carbonyl)-fumagillol, COL-3, combretastatin, combretastatin A4 Phosphate, Dalteparin, EMD 121974 (Cilengitide), endostatin, erlotinib, gefitinib (Iressa), genistein, halofuginone hydrobromide, Id1, Id3, IM862, imatinib mesylate, IMC-IC11 Inducible protein 10, interferon-alpha, interleukin 12, lavendustin A, LY317615 or AE-941, marimastat, mspin, medroxpregesterone acetate, Meth-1, Meth-2,2-methoxyestradiol (2-ME), neovastat, oteopontin cleaved product, PEX, pigment epithelium growth factor (PEGF), platelet factor 4, prolactin fragment, proliferin-related protein (PRP), PTK787/ZK 222584, ZD6474, recombinant human platelet factor 4 (rPF4), restin, squalamine, SU5416, SU6668, SU11248 suramin, Taxol, Tecogalan, thalidomide, thrombospondin, TNP-470, troponin-1, vasostatin, VEG1, VEGF-Trap, and ZD6474.

Non-limiting examples of angiogenesis inhibitors also include, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, pentosan polysulfate, angiotensin II antagonists, cyclooxygenase inhibitors (including non-steroidal anti-inflammatory drugs (NSAIDs) such as aspirin and ibuprofen, as well as selective cyclooxygenase-2 inhibitors such as celecoxib and rofecoxib), and steroidal anti-inflammatories (such as corticosteroids, mineralocorticoids, dexamethasone, prednisone, prednisolone, methylpred, betamethasone).

Other therapeutic agents that modulate or inhibit angiogenesis and may also be used in combination with the present compositions include agents that modulate or inhibit the coagulation and fibrinolysis systems. Examples of such agents that modulate or inhibit the coagulation and fibrinolysis pathways include, but are not limited to, heparin, low molecular weight heparins and carboxypeptidase U inhibitors (also known as inhibitors of active thrombin activatable fibrinolysis inhibitor [TAFIa]). U.S. Patent Publication No. 20090328239. U.S. Pat. No. 7,638,549.

The present composition may be administered by any method known in the art, including, without limitation, intranasal, oral, ocular, intraperitoneal, inhalation, intravenous, ICV, intracisternal injection or infusion, subcutaneous, implant, vaginal, sublingual, urethral (e.g., urethral suppository), subcutaneous, intramuscular, intravenous, transdermal, rectal, sub-lingual, mucosal, ophthalmic, spinal, intrathecal, intra-articular, intra-arterial, sub-arachinoid, bronchial and lymphatic administration. Topical formulation may be in the form of gel, ointment, cream, aerosol, etc.; intranasal formulation can be delivered as a spray or in a drop; transdermal formulation may be administered via a transdermal patch or iontorphoresis; inhalation formulation can be delivered using a nebulizer or similar device. Compositions can also take the form of tablets, pills, capsules, semisolids, powders, sustained release formulations, solutions, suspensions, elixirs, aerosols, or any other appropriate compositions.

To prepare such pharmaceutical compositions, the polynucleotide, POH, and/or an Argonaute protein (e.g., Ago-2) or the variant thereof may be mixed with a pharmaceutical acceptable carrier, adjuvant and/or excipient, according to conventional pharmaceutical compounding techniques. Pharmaceutically acceptable carriers that can be used in the present compositions encompass any of the standard pharmaceutical carriers, such as a phosphate buffered saline solution, water, and emulsions, such as an oil/water or water/oil emulsion, and various types of wetting agents. The compositions can additionally contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol, and various oils, including those of petroleum, animal, vegetable, or synthetic origin, e.g., peanut oil, soybean oil, mineral oil, sesame oil, etc. Liquid carriers, particularly for injectable solutions, include water, saline, aqueous dextrose, and glycols. For examples of carriers, stabilizers, and adjuvants, see Remington's Pharmaceutical Sciences, edited by E. W. Martin (Mack Publishing Company, 18th ed., 1990). The compositions also can include stabilizers and preservatives.

As used herein, the term “therapeutically effective amount” is an amount sufficient to treat a specified disorder or disease or alternatively to obtain a pharmacological response treating a disorder or disease. Methods of determining the most effective means and dosage of administration can vary with the composition used for therapy, the purpose of the therapy, the target cell being treated, and the subject being treated. Treatment dosages generally may be titrated to optimize safety and efficacy. Single or multiple administrations can be carried out with the dose level and pattern being selected by the treating physician. Suitable dosage formulations and methods of administering the agents can be readily determined by those of skill in the art. For example, the composition is administered at about 0.01 mg/kg to about 200 mg/kg, about 0.1 mg/kg to about 100 mg/kg, or about 0.5 mg/kg to about 50 mg/kg. When the compounds described herein are co-administered with another agent or therapy, the effective amount may be less than when the agent is used alone.

The compositions may be formulated for intranasal administration. The composition may be administered intranasally in a liquid form such as a solution, an emulsion, a suspension, drops, or in a solid form such as a powder, gel, or ointment. Devices to deliver intranasal medications are well known in the art. Nasal drug delivery can be carried out using devices including, but not limited to, intranasal inhalers, intranasal spray devices, atomizers, nasal spray bottles, unit dose containers, pumps, droppers, squeeze bottles, nebulizers, metered dose inhalers (MDI), pressurized dose inhalers, insufflators, and bi-directional devices. The nasal delivery device can be metered to administer an accurate effective dosage amount to the nasal cavity. The nasal delivery device can be for single unit delivery or multiple unit delivery. In a specific example, the ViaNase Electronic Atomizer from Kurve Technology (Bethell, Wash.) can be used in this invention (http://www.kurvetech.com). The compounds of the present invention may also be delivered through a tube, a catheter, a syringe, a packtail, a pledget, a nasal tampon or by submucosal infusion. U.S. Patent Publication Nos. 20090326275, 20090291894, 20090281522 and 20090317377.

The compositions can be formulated as aerosols using standard procedures. The compositions may be formulated with or without solvents and formulated with or without carriers. The formulation may be a solution or may be an aqueous emulsion with one or more surfactants. For example, an aerosol spray may be generated from pressurized container with a suitable propellant such as, dichlorodifluoromethane, trichlorofluoromethane, dichlorotetrafluoroethane, hydrocarbons, compressed air, nitrogen, carbon dioxide, or other suitable gas. The dosage unit can be determined by providing a valve to deliver a metered amount. Pump spray dispensers can dispense a metered dose or a dose having a specific particle or droplet size. As used herein, the term “aerosol” refers to a suspension of fine solid particles or liquid solution droplets in a gas. Specifically, aerosol includes a gas-borne suspension of droplets of a monoterpene (or sesquiterpene), as may be produced in any suitable device, such as an MDI, a nebulizer, or a mist sprayer. Aerosol also includes a dry powder composition of the composition of the instant invention suspended in air or other carrier gas. (Gonda (1990) Critical Reviews in Therapeutic Drug Carrier Systems 6:273-313. Raeburn et al., (1992) Pharmacol. Toxicol. Methods 27:143-159).

The compositions may be delivered to the nasal cavity as a powder in a form such as microspheres delivered by a nasal insufflator. The compositions may be absorbed to a solid surface, for example, a carrier. The powder or microspheres may be administered in a dry, air-dispensable form. The powder or microspheres may be stored in a container of the insufflator. Alternatively, the powder or microspheres may be filled into a capsule, such as a gelatin capsule, or other single dose unit adapted for nasal administration.

The pharmaceutical composition can be delivered to the nasal cavity by direct placement of the composition in the nasal cavity, for example, in the form of a gel, an ointment, a nasal emulsion, a lotion, a cream, a nasal tampon, a dropper, or a bioadhesive strip. In certain embodiments, it can be desirable to prolong the residence time of the pharmaceutical composition in the nasal cavity, for example, to enhance absorption. Thus, the pharmaceutical composition can optionally be formulated with a bioadhesive polymer, a gum (e.g., xanthan gum), chitosan (e.g., highly purified cationic polysaccharide), pectin (or any carbohydrate that thickens like a gel or emulsifies when applied to nasal mucosa), a microsphere (e.g., starch, albumin, dextran, cyclodextrin), gelatin, a liposome, carbamer, polyvinyl alcohol, alginate, acacia, chitosans and/or cellulose (e.g., methyl or propyl; hydroxyl or carboxy; carboxymethyl or hydroxylpropyl).

The composition can be administered by oral inhalation into the respiratory tract, i.e., the lungs.

Typical delivery systems for inhalable agents include nebulizer inhalers, dry powder inhalers (DPI), and metered-dose inhalers (MDI).

Nebulizer devices produce a stream of high velocity air that causes a therapeutic agent in the form of liquid to spray as a mist. The therapeutic agent is formulated in a liquid form such as a solution or a suspension of particles of suitable size. In one embodiment, the particles are micronized. The term “micronized” is defined as having about 90% or more of the particles with a diameter of less than about 10 μm. Suitable nebulizer devices are provided commercially, for example, by PART GmbH (Starnberg, Germany). Other nebulizer devices include Respimat (Boehringer Ingelheim) and those disclosed in, for example, U.S. Pat. Nos. 7,568,480 and 6,123,068, and WO 97/12687. The compositions can be formulated for use in a nebulizer device as an aqueous solution or as a liquid suspension.

DPI devices typically administer a therapeutic agent in the form of a free-flowing powder that can be dispersed in a patient's airstream during inspiration. DPI devices which use an external energy source may also be used in the present invention. To achieve a free-flowing powder, the therapeutic agent can be formulated with a suitable excipient (e.g., lactose). A dry powder formulation can be made, for example, by combining dry lactose having a particle size between about 1 μm and 100 μm with micronized particles and dry blending. Alternatively, the compositions can be formulated without excipients. The formulation is loaded into a dry powder dispenser, or into inhalation cartridges or capsules for use with a dry powder delivery device. Examples of DPI devices provided commercially include Diskhaler (GlaxoSmithKline, Research Triangle Park, N.C.) (see, e.g., U.S. Pat. No. 5,035,237); Diskus (GlaxoSmithKline) (see, e.g., U.S. Pat. No. 6,378,519; Turbuhaler (AstraZeneca, Wilmington, Del.) (see, e.g., U.S. Pat. No. 4,524,769); and Rotahaler (GlaxoSmithKline) (see, e.g., U.S. Pat. No. 4,353,365). Further examples of suitable DPI devices are described in U.S. Pat. Nos. 5,415,162, 5,239,993, and 5,715,810 and references therein.

MDI devices typically discharge a measured amount of therapeutic agent using compressed propellant gas. Formulations for MDI administration include a solution or suspension of active ingredient in a liquefied propellant. Examples of propellants include hydrofluoroalklanes (HFA), such as 1,1,1,2-tetrafluoroethane (HFA 134a) and 1,1,1,2,3,3,3-heptafluoro-n-propane, (HFA 227), and chlorofluorocarbons, such as CCl.sub.3F. Additional components of HFA formulations for MDI administration include co-solvents, such as ethanol, pentane, water; and surfactants, such as sorbitan trioleate, oleic acid, lecithin, and glycerin. (See, for example, U.S. Pat. No. 5,225,183, EP 0717987, and WO 92/22286). The formulation is loaded into an aerosol canister, which forms a portion of an MDI device. Examples of MDI devices developed specifically for use with HFA propellants are provided in U.S. Pat. Nos. 6,006,745 and 6,143,227. For examples of processes of preparing suitable formulations and devices suitable for inhalation dosing see U.S. Pat. Nos. 6,268,533, 5,983,956, 5,874,063, and 6,221,398, and WO 99/53901, WO 00/61108, WO 99/55319, and WO 00/30614.

The compositions may be encapsulated in liposomes or microcapsules for delivery via inhalation. A liposome is a vesicle composed of a lipid bilayer membrane and an aqueous interior. The lipid membrane may be made of phospholipids, examples of which include phosphatidylcholine such as lecithin and lysolecithin; acidic phospholipids such as phosphatidylserine and phosphatidylglycerol; and sphingophospholipids such as phosphatidylethanolamine and sphingomyelin. Alternatively, cholesterol may be added. A microcapsule is a particle coated with a coating material. For example, the coating material may consist of a mixture of a film-forming polymer, a hydrophobic plasticizer, a surface activating agent, or/and a lubricant nitrogen-containing polymer. U.S. Pat. Nos. 6,313,176 and 7,563,768.

The compositions may be formulated for ocular administration. For ocular administration, the compositions described herein can be formulated as a solution, emulsion, suspension, etc. A variety of vehicles suitable for administering compounds to the eye are known in the art. Specific non-limiting examples are described in U.S. Pat. Nos. 6,261,547; 6,197,934; 6,056,950; 5,800,807; 5,776,445; 5,698,219; 5,521,222; 5,403,841; 5,077,033; 4,882,150; and 4,738,851.

The compositions can be given alone or in combination with other drugs for the treatment of the above diseases for a short or prolonged period. The present compositions can be administered to a mammal, preferably a human. Mammals include, but are not limited to, murines, rats, rabbit, simians, bovines, ovine, porcine, canines, feline, farm animals, sport animals, pets, equine, and primates.

A “cancer” or “tumor” as used herein refers to an uncontrolled growth of cells which interferes with the normal functioning of the bodily organs and systems, and/or all neoplastic cell growth and proliferation, whether malignant or benign, and all pre-cancerous and cancerous cells and tissues. A subject that has a cancer or a tumor is a subject having objectively measurable cancer cells present in the subject's body. Included in this definition are benign and malignant cancers, as well as dormant tumors or micrometastatses. Cancers which migrate from their original location and seed vital organs can eventually lead to the death of the subject through the functional deterioration of the affected organs. As used herein, the term “invasive” refers to the ability to infiltrate and destroy surrounding tissue. Melanoma is an invasive form of skin tumor. As used herein, the term “carcinoma” refers to a cancer arising from epithelial cells. Examples of cancer include, but are not limited to, brain tumor, nerve sheath tumor, breast cancer, colon cancer, carcinoma, lung cancer, hepatocellular cancer, gastric cancer, pancreatic cancer, cervical cancer, ovarian cancer, liver cancer, bladder cancer, cancer of the urinary tract, thyroid cancer, renal cancer, renal cell carcinoma, carcinoma, melanoma, head and neck cancer, brain cancer, and prostate cancer, including but not limited to androgen-dependent prostate cancer and androgen-independent prostate cancer. Examples of brain tumor include, but are not limited to, benign brain tumor, malignant brain tumor, primary brain tumor, secondary brain tumor, metastatic brain tumor, glioma, glioblastoma multiforme (GBM), medulloblastoma, ependymoma, astrocytoma, pilocytic astrocytoma, oligodendroglioma, brainstem glioma, optic nerve glioma, mixed glioma such as oligoastrocytoma, low-grade glioma, high-grade glioma, supratentorial glioma, infratentorial glioma, pontine glioma, meningioma, pituitary adenoma, and nerve sheath tumor.

“Conditions” and “disease conditions,” as used herein may include, but are in no way limited to any form of neurovascular diseases, any form of malignant neoplastic cell proliferative diseases, and abnormal angiogenesis (e.g., tumor angiogenesis, insufficient angiogenesis, or excessive angiogenesis). Examples of neurovascular diseases include but are not limited to stroke, brain trauma, AVM, brain aneurysms, carotid disease, cervical artery dissection, and vascular malformations. Examples of malignant neoplastic cell proliferative diseases include but are not limited to cancer and tumor. Examples of cancer and tumor include, but are not limited to, brain tumor, breast cancer, colon cancer, carcinoma, lung cancer, hepatocellular cancer, gastric cancer, pancreatic cancer, cervical cancer, ovarian cancer, liver cancer, bladder cancer, cancer of the urinary tract, thyroid cancer, renal cancer, renal cell carcinoma, carcinoma, melanoma, head and neck cancer, brain cancer, and prostate cancer, including but not limited to androgen-dependent prostate cancer and androgen-independent prostate cancer.

The term “functional” when used in conjunction with “equivalent”, “analog”, “derivative” or “variant” or “fragment” refers to an entity or molecule which possess a biological activity that is substantially like a biological activity of the entity or molecule of which it is an equivalent, analog, derivative, variant, or fragment thereof.

As used herein, a “subject” means a human or animal. Usually, the animal is a vertebrate such as a primate, rodent, domestic animal, or game animal Primates include chimpanzees, cynomologous monkeys, spider monkeys, and macaques, e.g., Rhesus. Rodents include mice, rats, woodchucks, ferrets, rabbits, and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets.

“Mammal” as used herein refers to any member of the class Mammalia, including, without limitation, humans, and nonhuman primates such as chimpanzees and other apes and monkey species; farm animals such as cattle, sheep, pigs, goats, and horses; domestic mammals such as dogs and cats; laboratory animals including rodents such as mice, rats and guinea pigs, and the like. The term does not denote a particular age or sex. Thus, adult, and newborn subjects, as well as fetuses, whether male or female, are intended to be included within the scope of this term.

A subject can be one who has been previously diagnosed with or identified as suffering from or having a condition in need of treatment (e.g., brain tumors) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition, or one or more complications related to the condition or a subject who does not exhibit risk factors. A “subject in need” of treatment for a particular condition can be a subject suspected of having that condition, diagnosed as having that condition, already treated, or being treated for that condition, not treated for that condition, or at risk of developing that condition.

As used herein, “variants” can include, but are not limited to, those that include conservative amino acid mutations, SNP variants, splicing variants, degenerate variants, and biologically active portions of a gene. A “degenerate variant” as used herein refers to a variant that has a mutated nucleotide sequence, but still encodes the same polypeptide due to the redundancy of the genetic code. In accordance with the present invention, the Argonaute protein (e.g., Ago-2) may be modified, for example, to facilitate or improve identification, expression, isolation, storage and/or administration, so long as such modifications do not reduce Argonaute's function to unacceptable level. In various embodiments, a variant of the Argonaute protein has at least 10%, 20%, 30%, 40%, 50%, 60%, 70%, 80%, 90%, or 95% of the function of a wild-type Argonaute protein.

Cerebral arteriovenous malformation (AVM) is a vascular disease exhibiting abnormal blood vessel morphology and function. Current medical treatments for cerebrovascular disorders involve highly invasive procedures such as microsurgery, stereotactic radiosurgery and/or endovascular embolization. Moreover, difficult access to the brain region of interest (e.g., AVM nidus) can represent a significant risk to nearby eloquent cortical, subcortical, and neurovascular structures. Although these therapies are traditionally considered curative, AVM may recur, underlining the importance for the development of more efficient and safer therapies. The use of pharmaceutical drugs faces an important challenge which is the successful crossing of the blood-brain barrier (BBB), responsible for the low efficacy of drugs given systemically.

Exogenous application of miR-18a ameliorates the abnormal characteristics of AVM-derived brain endothelial cells (AVM-BEC) without the use of transfection reagents. In this application, we identify the mechanisms by which mir-18a is internalized by AVM-BEC and explore the clinical application of a systemic miRNA carrier.

Primary cultures of AVM-BEC were isolated from surgical specimens and tested for endogenous miR-18a levels using qPCR. Conditioned media (CM) was derived from AVM-BEC cultures (AVM-BEC-CM). Ago-2 was detected using western blotting and immunostaining techniques. Secreted products (e.g., thrombospondin-1 (TSP-1)) were tested using ELISA. In the in vivo angiogenesis glioma model, animals were treated with miR-18a in combination with Ago-2. Plasma was obtained, and tested for TSP-1 and vascular endothelial growth factor (VEGF)-A.

AVM-BEC-CM significantly enhanced miR-18a internalization. Ago-2 was highly expressed in AVM-BEC; and siAgo-2 decreased miR-18a entry into brain-derived endothelial cells. Only brain-derived endothelial cells were responsive to the Ago-2/miR-18a complex and no other cell types tested. Brain endothelial cells treated with the Ago-2/miR-18a complex in vitro increased TSP-1 secretion. Using an in vivo angiogenesis model, the effects of the Ago-2/miR-18a complex caused a significant increase in TSP-1 and decrease in VEGF-A secretion in the plasma.

The functional effects of miR-18a on brain endothelial cells depend heavily on the presence of Ago-2. Without wishing to be bound by any theory, the requirement for this binary complex implies the existence/assembly of a putative cell membrane receptor for the ribonucleoprotein complex prior to internalization. Our studies found that brain endothelial cells are highly permissive to miRNA uptake compared to other endothelial cell types, such as the human microvascular endothelial cell line-1 (HMEC-1) and human umbilical vascular endothelial cells (HUVEC). Without wishing to be bound by any theory, this may be due to an intrinsic property of brain endothelial cells or is related to differences in ribonucleoprotein receptor density among the different endothelial cell types.

Taken together, Ago-2 facilitates miR-18a entry into AVM-brain endothelial cells in vitro and in vivo. Thus far, Ago-2 has been identified as an intracellular component of the RNA-induced silencing complex (RISC). However, we are the first to show that Ago-2 can be used as a specific miRNA carrier, particularly to the brain vasculature, with functional effects. This study demonstrates the clinical application of Ago-2 as a miRNA delivery platform for the treatment of brain vascular diseases.

The use of Ago-2 as a miRNA carrier and stabilizer overcomes all the limitations of systemic miRNA delivery by being able to carry functional miRNA specifically to the brain, thus traversing the BBB. The delivery of Ago-2/miRNA complex has significantly high target specificity and no apparent off-target effects while being minimally invasive (e.g., intravenous, or intranasal administration).

### Argonaute Proteins

Argonaute proteins make up a highly conserved family whose members play a central role in RNA silencing processes as essential catalytic components of the RNA-induced silencing complex (RISC).

Non-limiting examples of Argonaute proteins include, but are not limited to, Ago-1, Ago-2, Ago-3, Ago-4, PIWIL1, PIWIL 2, PIWIL 3 or PIWIL 4. In some embodiments, the Argonaute protein is an Ago-like protein or a Piwi-like protein. Descriptions of Argonaute proteins, including Ago-like and Piwi-like proteins are discussed in Tolia N. H. et al. Slicer and the Argonautes. 3(1), 2007, p. 36-43, which is incorporated by reference herein.

In some cases, a subject Ago polypeptide is a naturally occurring polypeptide (e.g., naturally occurs in bacterial and/or archaeal cells). In other cases, a subject Ago polypeptide is not a naturally occurring polypeptide.

Argonaute (Ago) proteins are composed of at least four recognized domains: (i) an amino-terminal (N-domain); (ii) a PAZ (PIWI/Argonaute/Zwille) domain; (iii) a MID (middle) domain; and (iv) a PIWI (P-element-induced whimpy testes) domain. Certain Ago proteins bind and utilize guide RNAs with a strong preference for a 5′-phosphate group. Crystal structures of exemplary eukaryotic and prokaryotic Ago MID domains have been described. For example, the human Ago MID domain structure provides a structural basis for the 5′-nucleotide recognition of the guide RNA observed in eukaryotic Agos. Based on existing crystal structures, the phosphorylated 5′-end of the guide RNA is localized in the MID-PIWI domain interface with the 3′-end anchored to the PAZ domain. On binding to mRNA, the catalytic RNase H-like active site located in the PIWI domain is in position to cleave the targeted mRNA.

In some embodiments, the Argonaute comprises at least 30% amino acid identity to a prokaryotic Argonaute. In some embodiments, the Argonaute comprises at least 30% amino acid identity to a bacterial Argonaute. In some embodiments, the Argonaute comprises at least 30% amino acid identity to an archaeal Argonaute. In some embodiments, the Argonaute comprises at least 30% amino acid identity to an Argonaute from a mesophile. In some embodiments, the Argonaute comprises at least 30% amino acid identity to an Argonaute from a thermophile. In some embodiments, the Argonaute comprises at least 30% amino acid identity to an Argonaute from a species selected from the group consisting of: Thermus thermophilus, Thermus thermophilus JL-18, Thermus thermophilus strain HB27, Aquifex aeolicus strain VF5, Pyrococcus furiosus, Archaeoglobus fulgidus, Anoxybacillus flavithermus, Halogeometricum borinquense, Microsystis aeruginosa, Clostridium bartlettii, Halorubrum lacusprofundi, Thermosynechococcus elongatus, and Synechococcus elongatus, or any combination thereof. In some embodiments, the Argonaute comprises at least 30% amino acid identity to an Argonaute from T. thermophilus.

In our studies we show that Ago-2, a RNA-binding protein, forms a stable ribonucleoprotein complex with miRNA and can be used as an exogenous clinically relevant agent. This Ago-2/miRNA complex is 1) internalized specifically by brain endothelial cells, 2) delivers functional miRNA, and 3) is clinically relevant based on in vivo activity. Ago-2 enhances miRNA stability allowing a more efficient miRNA internalization and consequent increased release of growth factors, e.g., thrombospondin-1 (TSP-1). TSP-1 is a key anti-angiogenic factor that antagonizes another pivotal molecule, vascular endothelial growth factor-A (VEGF-A). In vivo, the Ago-2/miRNA complex was administered systemically and effectively “normalized” the expression of key angiogenic factors to control plasma levels. Since the Ago-2/miRNA complex targets specifically the brain vasculature it has significant clinical relevance in the treatment of cerebrovascular diseases such as brain arteriovenous malformations (AVM), or diseases that involve active angiogenesis (i.e., stroke, angiogenesis in brain tumors). Furthermore, since Ago-2 is found in human circulation it is a biocompatible agent and therefore less likely to induce toxic side effects. Furthermore, Ago-2 can bind to several different miRNA; thus, function will be related to activity of the miRNA. Hence, this carrier can be used for carrying a variety of miRNA sequences, and therefore target a variety of systems regulated by miRNA (e.g., growth factor expression, tumor suppression, neuronal development, cell differentiation and proliferation, immune system cell regulation). This opens new perspectives for the use of Ago-2 as a stable, safe, and biocompatible miRNA carrier in different diseases. The current problem with miRNA-based therapy is inefficient delivery to the intended target tissue, off-target effects of miRNA and toxicity of the miRNA modulator (Noori-Daloii and Nejatizadeh; 2011). The delivery of miRNA with Ago-2 bypasses all these issues and can efficiently be administered through the intravenous or the intranasal route, as shown by our in vivo studies. For the reasons mentioned above, Ago-2/miRNA treatment is a safe and efficient therapeutic approach that can be used in the clinic.

MiRNA are small non-coding RNA that regulates protein expression by targeting messenger RNA for cleavage or translational repression. MiRNA-based therapy has great potential but faces several physiological obstacles. However, without wishing to be bound by any theory, it is believed that the use of Ago-2 as a miRNA carrier offers several advantages:

1) Ago-2 protects miRNA from intravascular degradation—intravenous naked delivery of miRNA often leads to degradation or renal clearance, meaning that the kidneys and other highly vascularized organs are preferred targets for this approach. Other research groups have tried chemical modification of these oligoribonucleotides for stabilization, but they have low membrane penetration efficacy. Another alternative is the use of nanoparticle carriers; however, nanoparticles are often trapped by the reticuloendothelial system in the liver, lung, and bone marrow, resulting in degradation by activated immune cells. Also, the physical and chemical properties of the nanoparticle surface can lead to hemolysis, thrombogenicity and complement activation, resulting in altered biodistribution and potential toxicity.

2) Ago-2 specifically and efficiently delivers miRNA to the endothelial cells of the brain vasculature—miRNA alone has low tissue penetrance and poor intracellular delivery, which can be overcome by using of transfection reagents e.g., lipofectamine (highly toxic in vivo), structural alterations of the miRNA (which offer low tissue penetrance), and nanoparticle/vesicle encapsulation. Many nanoparticles are internalized by endocytosis which can lead to miRNA degradation because lysosomes, which have an acidified (pH ^{˜}4.5) contain nucleases.

3) Ago-2 complex formation does not require modification of miRNA thus function is maintained—chemical modification of miRNA such as 2′-O-methylation of the lead strand, intended to decrease intravascular degradation and immune system activation, lowers off-target effects without loss of activity but has poor internalization efficiency.

The growing number of miRNA sequences and their functions opens new perspectives of treatment for the use of a stable, safe, and biocompatible miRNA carrier. Therefore, our invention has strong therapy-based applications for the treatment of cerebrovascular disorders, stroke, and brain tumors, which depend largely on the regulation of angiogenesis (formation of new blood vessels).

### Combination Therapy

Various method described herein can further comprise providing and administering a therapeutically effective amount of an anti-angiogenic drug to the subject. In various embodiments, the mixture and the anti-angiogenic drug are administered concurrently or sequentially. In various embodiments, the mixture is administered before, during or after administering the anti-angiogenic drug. As a non-limiting example, the mixture may be administered, for example, daily at the dosages, and the anti-angiogenic drug may be administered, for example, daily, weekly, biweekly, every fortnight and/or monthly at the dosages. As another non-limiting example, the mixture may be administered, for example, daily, weekly, biweekly, every fortnight and/or monthly, at the dosages, and the anti-angiogenic drug may be administered, for example, daily at the dosages. Further, each of the mixture and the anti-angiogenic drug may be administered daily, weekly, biweekly, every fortnight and/or monthly, wherein the mixture is administered at the dosages on a day different than the day on which the anti-angiogenic drug is administered at the dosages. In some embodiments, the mixture and the anti-angiogenic drug are in one composition or separate compositions.

In accordance with the present invention, examples of anti-angiogenic drugs include but are not limited to Genentech/Roche (Bevacizumab/Avastin®), Bayer and Onyx Pharmaceuticals (sorafenib/Nexavar®), Pfizer (sutinib/Sutent®), GlaxoSmithKline (pazopanib/Votrient®), Novartis (everolimus/Affinitor®), Celgene (pomalidomide/Pomalyst®) and Ipsen and Active Biotech (tasquinimod/ABR-215050, CID 54682876).

Various method described herein can further comprise providing and administering a therapeutically effective amount of a chemotherapeutic agent to the subject. In accordance with the invention, the mixture and the chemotherapeutic agent are administered concurrently or sequentially. Still in accordance with the invention, the mixture is administered before, during or after administering the chemotherapeutic agent. As a non-limiting example, the mixture may be administered, for example, daily at the dosages, and the chemotherapeutic agent may be administered, for example, daily, weekly, biweekly, every fortnight and/or monthly at the dosages. As another non-limiting example, the mixture may be administered, for example, daily, weekly, biweekly, every fortnight and/or monthly, at the dosages, and the chemotherapeutic agent may be administered, for example, daily at the dosages. Further, each of the mixture and the chemotherapeutic agent may be administered daily, weekly, biweekly, every fortnight and/or monthly, wherein the mixture is administered at the dosages on a day different than the day on which the chemotherapeutic agent is administered at the dosages. In some embodiments, the mixture and the chemotherapeutic agent are in one composition or separate compositions.

In accordance with the present invention, examples of the chemotherapeutic agent include but are not limited to Temozolomide, Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Ipilimumab, Irinotecan, Mechlorethamine, Melphalan, Mercaptopurine, Methotrexate, Mitoxantrone, Ocrelizumab, Ofatumumab, Oxaliplatin, Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, ixabepilone, Estramustine, prednisone, methylprednisolone, dexamethasone or a combination thereof.

### Pharmaceutical Compositions

In accordance with the present invention, various compositions described herein may be used for delivering a polynucleotide to a cell, inhibiting angiogenesis, promoting angiogenesis, and/or treating, preventing, reducing the likelihood of having, reducing the severity of and/or slowing the progression of a condition in a subject.

In various embodiments, the angiogenesis is angiogenesis in brain. In various embodiments, the angiogenesis is tumor angiogenesis. In various embodiments, the condition is a neurovascular disease. In various embodiments, the condition is cerebral arteriovenous malformations (AVM) or stroke. In various embodiments, the condition is a tumor. In various embodiments, the condition is brain tumor, glioma, glioblastoma, and/or glioblastoma multiforme (GBM). In certain embodiments, the composition is administered to a human.

In various embodiments, the miRNA is miR-18a, miR-133b or miR-128a. In some embodiments, the miRNA is a miRNA suppressing angiogenesis (e.g., miR-92, miR-92a, miR-221/22). In various embodiments, the composition comprises about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 nmol/L miRNA.

In various embodiments, the Ago-2 can be a wild-type Ago-2 or recombinant Ago-2. In various embodiments, the variant of Ago-2 is a functional variant, equivalent, analog, derivative, or salt of Ago-2. In various embodiments, the Ago-2 or the variant thereof can be from any source, e.g., rat, mouse, guinea pig, dog, cat, rabbit, pig, cow, horse, goat, donkey, or human. In various embodiments, the composition comprises about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 nmol/L Ago-2 or a variant thereof.

In various embodiments, the composition further comprises an anti-angiogenic drug. In various embodiments, the composition further comprises a chemotherapeutic agent.

In accordance with the invention, the miRNA and the Ago-2 or the variant thereof useful in the treatment of disease in mammals will often be prepared substantially free of naturally occurring immunoglobulins or other biological molecules. Preferred miRNAs and/or Ago-2s or variants thereof will also exhibit minimal toxicity when administered to a mammal.

In various embodiments, the pharmaceutical compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral, enteral, topical, or local. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art. In various embodiments, the composition is formulated for intratumoral, intracranial, intraventricular, intrathecal, epidural, intradural, intravascular, intravenous, intraarterial, intramuscular, subcutaneous, intraperitoneal, intranasal, or oral administration.

In various embodiments, the composition is administered 1-3 times per day, 1-7 times per week, or 1-9 times per month. In various embodiments, the composition is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. In various embodiments, the composition is administered once a day (SID/QD), twice a day (BID), three times a day (TID), four times a day (QID), or more, to administer an effective amount of the miRNA and the Ago-2 or the variant thereof to the subject, where the effective amount is any one or more of the doses described herein.

In various embodiments, the pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted, or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols, and water. Solid carriers include starch, lactose, calcium sulfate, dihydrate, terra alba, magnesium stearate or stearic acid, talc, pectin, acacia, agar, or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing, and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in each subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Before administration to patients, formulants may be added to the composition. A liquid formulation may be preferred. For example, these formulants may include oils, polymers, vitamins, carbohydrates, amino acids, salts, buffers, albumin, surfactants, bulking agents, or combinations thereof.

Carbohydrate formulants include sugar or sugar alcohols such as monosaccharides, disaccharides, or polysaccharides, or water-soluble glucans. The saccharides or glucans can include fructose, dextrose, lactose, glucose, mannose, sorbose, xylose, maltose, sucrose, dextran, pullulan, dextrin, alpha, and beta cyclodextrin, soluble starch, hydroxyethyl starch and carboxymethylcellulose, or mixtures thereof. “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used if the sugar or sugar alcohol is soluble in the aqueous preparation. In one embodiment, the sugar or sugar alcohol concentration is between 1.0 w/v % and 7.0 w/v %, preferable between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

Another drug delivery system for increasing circulatory half-life is the liposome. Methods of preparing liposome delivery systems are discussed in (Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277).

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

The compositions of the invention may be sterilized by conventional, well-known sterilization techniques. The resulting solutions may be packaged for use or filtered under aseptic conditions and lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and stabilizers (e.g., 1-20% maltose, etc.).

### Kits

The present disclosure provides for a kit comprising: (a) a polynucleotide; (b) perillyl alcohol (POH); and (c) instructions for delivering the polynucleotide to a cell or a subject using POH.

The present disclosure provides for a kit comprising: (a) a polynucleotide; (b) perillyl alcohol (POH); (c) an Argonaute protein or a variant thereof; and (d) instructions for delivering the polynucleotide to a cell or a subject using the POH and the Argonaute protein or a variant thereof.

In various embodiments, the present invention provides a kit for inhibiting angiogenesis in a subject.

In various embodiments, the present invention provides a kit for treating, preventing, reducing the severity of and/or slowing the progression of a condition in a subject.

In various embodiments, the kits described herein can further comprise an anti-angiogenic drug and/or chemotherapeutic agent, and instructions for using the anti-angiogenic drug and/or chemotherapeutic agent to inhibit angiogenesis and/or to treat, prevent, reduce the likelihood of having, reduce the severity of and/or slow the progression of the condition in the subject.

The composition may be formulated for intranasal administration. The kit may comprise a device for intranasal administration of the composition. The device for intranasal administration may be an intranasal spray device, an atomizer, a nebulizer, a metered dose inhaler (MDI), a pressurized dose inhaler, an insufflator, an intranasal inhaler, a nasal spray bottle, a unit dose container, a pump, a dropper, a squeeze bottle, or a bi-directional device.

### EXAMPLES

The following examples are offered for illustrative purposes only and are not intended to limit the scope of the present invention in any way.

### Example 1 MicroRNA-18a Normalizes Brain Arteriovenous Malformations (AVM) by Inhibiting BMP4 and HIF-1α Pathways

Brain arteriovenous malformations (AVMs) are abnormal tangles of vessels where arteries and veins directly connect without intervening capillary nets, increasing the risk of intracerebral hemorrhage and stroke. Current treatments are highly invasive and often not feasible. Thus, effective non-invasive treatments are needed.

We previously showed that AVM brain endothelial cells (AVM-BEC) secreted higher levels of vascular endothelial growth factor (VEGF) and lower levels of thrombospondin-1 (TSP-1) than control BEC; and that miR-18a normalizes AVM-BEC function and phenotype without affecting control BEC. Here, we show that miR-18a increases TSP-1 and decreases VEGF activity through a reduction of plasminogen activator inhibitor (PAI-1/SERPINE1) levels. Furthermore, we have elucidated the mechanism of action of miR-18a by blocking bone morphogenetic protein 4 (BMP4) and hypoxia inducible factor 1α (HIF-1α) pathways in AVM both in vitro and in vivo. This leads to a decrease in BMP4/activin-like kinase 2 (ALK2)/ALK1/ALK5 and Notch pathways, without affecting BMP9 and TGF-β levels. MiR-18a also reduces the abnormal AVM-BEC invasiveness, which correlates with a decrease in MMP2, MMP9 and ADAM10.

In vivo pharmacokinetic studies showed that miR-18a reaches the brain following intravenous (IV) and intranasal (IN) administration. IN co-delivery of miR-18a and NEO100, a good manufacturing practices (GMP)-quality form of perillyl alcohol (POH), greatly improved the pharmacokinetic profile of miR-18a in the brain without affecting its pharmacologic properties. In an Mgp^{-/-} mouse model of AVM, miR-18a decreased abnormal cerebral vasculature, and restored the functionality of the bone marrow, lungs, spleen, and liver. These data suggest that miR-18a may have significant clinical value in preventing, reducing, and potentially reversing AVM.

Arteriovenous malformations (AVMs) are abnormal vascular networks where arteries are directly shunted to veins without an interposed capillary system. Although brain AVMs are considered rare, the actual prevalence rate may be higher, as only 12% are estimated to become symptomatic. This abnormal vasculature constitutes an important cause of intracerebral hemorrhaging in young adults, with a mortality rate of 10-15% and a morbidity rate of 30-50%. (Dupuis-Girod S, Ginon I, Saurin J, et al. Bevacizumab in patients with hereditary hemorrhagic telangiectasia and severe hepatic vascular malformations and high cardiac output. JAMA. 2012; 307(9):948-955). Current therapies include microsurgical excision, stereotactic radiosurgery and endovascular embolization, all of which are highly invasive and often not feasible depending on the location of the AVM nidus. (Lebrin F, Srun S, Raymond K, et al. Thalidomide stimulates vessel maturation and reduces epistaxis in individuals with hereditary hemorrhagic telangiectasia. Nat Med. 2010; 16:420-428).

Hence, an effective non-invasive treatment for brain AVM is sorely needed. Therapeutic approaches to block angiogenesis using bevacizumab² (Dupuis-Girod S, Ginon I, Saurin J, et al. Bevacizumab in patients with hereditary hemorrhagic telangiectasia and severe hepatic vascular malformations and high cardiac output. JAMA. 2012; 307(9):948-955) and thalidomide³ (Lebrin F, Srun S, Raymond K, et al. Thalidomide stimulates vessel maturation and reduces epistaxis in individuals with hereditary hemorrhagic telangiectasia. Nat Med. 2010; 16:420-428). have shown benefits in patients with Hereditary Hemorrhagic Telangiectasia (HHT), an autosomal dominant disorder that exhibits AVM as part of its clinical phenotype4. (Kim H, Su H, Weinsheimer S, Pawlikowska L, Young W L. Brain arteriovenous malformation pathogenesis: A response-to-injury paradigm. Acta Neurochir Suppl. 2011; 111:83-92). Although these studies have limitations like adverse effects or lack of randomization of the clinical trials, they raise hopes for identifying anti-angiogenic therapies for AVM treatment. Our previous studies showed that, compared to control brain endothelial cells (BEC), AVM-BEC secreted higher levels of the pro-angiogenic vascular endothelial growth factor (VEGF) and lower levels of the anti-angiogenic thrombospondin-1 (TSP-1)⁵. (Stapleton C J, Armstrong D L, Zidovetzki R, Liu C Y, Giannotta S L, Hofman F M. Thrombospondin-1 modulates the angiogenic phenotype of human cerebral arteriovenous malformation endothelial cells. Neurosurgery. 2011; 68(5):1342-1353). Treatment of AVM-BEC with miR-18a, a small non-coding RNA molecule involved in post-transcriptional regulation of gene expression, increased TSP-1 release and decreased VEGF levels in vitro and in vivo, suggesting a potential therapeutic use for miR-18a⁶. (Ferreira R, Santos T, Amar A, et al. MicroRNA-18a improves human cerebral arteriovenous malformation endothelial cell function. Stroke. 2014; 45(1):293-297).

In the current studies, we have elucidated the mechanism of action by which miR-18a normalizes the aberrant phenotype and function of AVM-BEC, via inhibition of bone morphogenetic protein 4 (BMP4) and hypoxia inducible factor 1α (HIF-1α). Namely, miR-18a decreases VEGF by reducing the levels of plasminogen activator inhibitor (PAI-1/SERPINE1) and the expression of BMP4/ALK2/ALK1/ALK5. This translates into a reduced activation of Smad1/5 and Notch signaling. The blockade of HIF-1α expression by miR-18a occurs under normoxia, the condition found in AVM^{7,8}, (Meyer B, Schaller C, Frenkel C, Ebeling B, Schramm J. Distributions of local oxygen saturation and its response to changes of mean arterial blood pressure in the cerebral cortex adjacent to arteriovenous malformations. Stroke. 1999; 30(12):2623-2630) (Taguchi A, Yanagisawa K, Tanaka M, et al. Identification of hypoxia-inducible factor-1α as a novel target for mir-17-92 microma cluster. Cancer Res. 2008; 68(14):5540-5545) leading to a stronger reduction of PAI-1 and VEGF in normoxic than in hypoxic conditions. MiR-18a specifically normalized AVM-BEC invasiveness, which correlated with a decrease in the levels of matrix metalloproteinase 2 (MMP2), MMP9 and ADAM metallopeptidase domain 10 (ADAM10).

In vivo pharmacokinetic studies using intravenous (IV), or intranasal (IN) delivery showed that miR-18a reaches the brain, with IN administration leading to an extended retention time. IN co-administration of miR-18a with NEO100, a good manufacturing practices (GMP)-quality form of perillyl alcohol (POH), accelerated miR-18a delivery and increased the amount of miRNA that reached the brain. In vivo efficacy studies using the Mgp^{-/-} AVM mouse model showed that miR-18a normalized the brain vasculature, and restored the compromised functionality of the bone marrow, lungs, spleen, and livers. Hence, we propose that miR-18a could show a significant clinical value in the treatment and prevention of brain AVMs.

### Methods

### Cell Culture

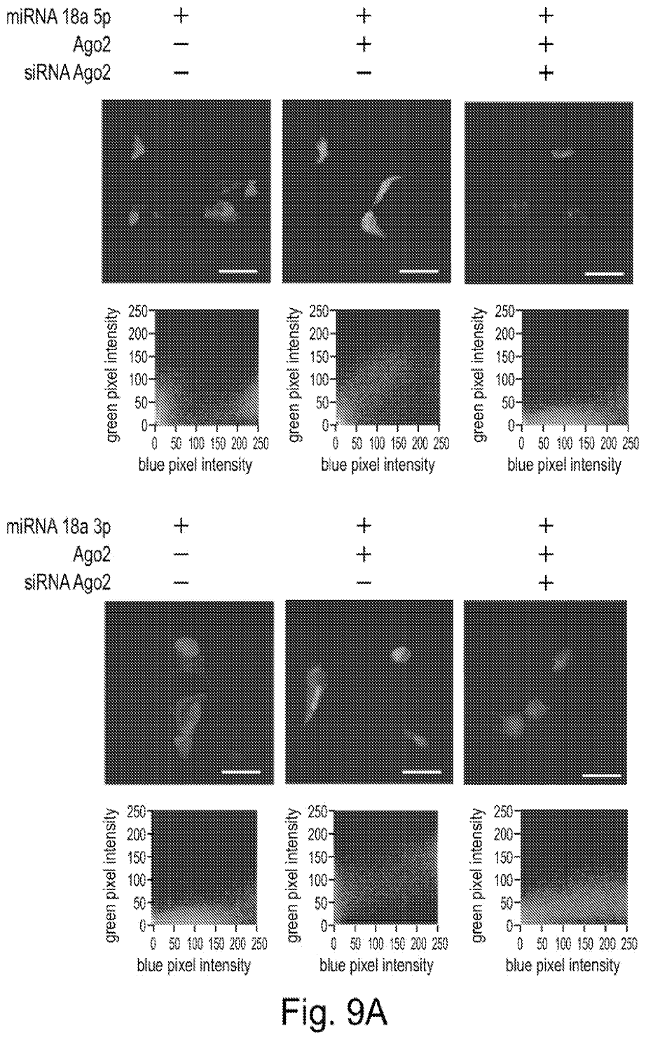

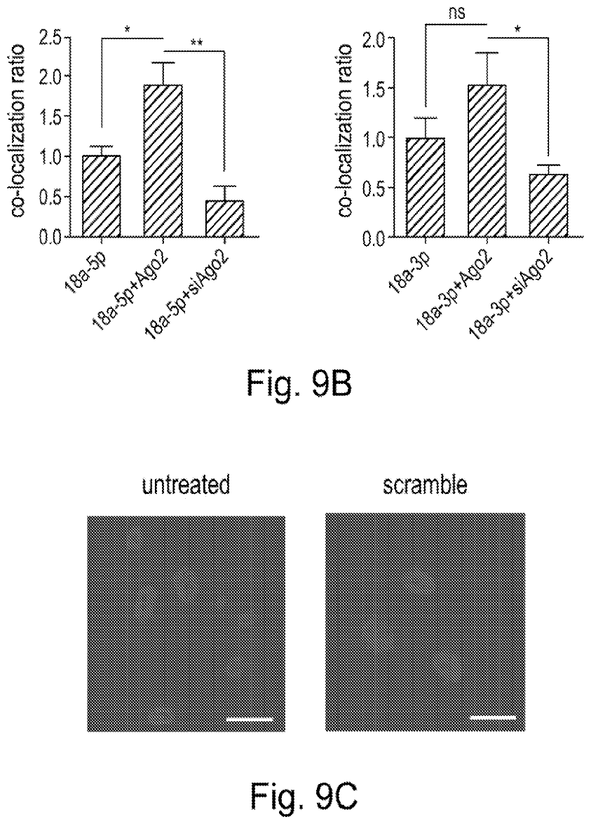

Human surgical specimens were obtained following written informed consent from patients in accordance with Declaration of Helsinki guidelines and the Institutional Review Board (HS-04B053), at Keck School of Medicine, USC. AVM-BECs were isolated from brain tissues of patients who underwent microsurgical AVM resection. Control BECs were isolated from the structurally normal complex of patients who underwent temporal lobectomy for intractable epilepsy. Endothelial cell (EC) isolation and culture were described^{5,9}. (Stapleton C J, Armstrong D L, Zidovetzki R, Liu C Y, Giannotta S L, Hofman F M. Thrombospondin-1 modulates the angiogenic phenotype of human cerebral arteriovenous malformation endothelial cells. Neurosurgery. 2011; 68(5):1342-1353) (Ferreira R, Santos T, Amar A, et al. Argonaute-2 promotes miR-18a entry in human brain endothelial cells. J Am Heart Assoc. 2014; 3(3):e000968) ECs were only used between passages 3-5. Experiments were performed under shear flow conditions (12 dyn/cm²) to reproduce arterial flow⁶. (Ferreira R, Santos T, Amar A, et al. MicroRNA-18a improves human cerebral arteriovenous malformation endothelial cell function. Stroke. 2014; 45(1):293-297). For hypoxic experiments, cells were cultured in a Galaxy 48R (Eppendorf) incubator at 3% O₂. MicroRNAs were co-administered with Ago-2 to increase the cellular uptake of miR-18a⁹ (Ferreira R, Santos T, Amar A, et al. Argonaute-2 promotes miR-18a entry in human brain endothelial cells. J Am Heart Assoc. 2014; 3(3):e000968) (FIG. 9). Methods regarding cell treatments and transfections were included in Data.

TABLE 1
Antibodies used for western blot studies.

| Antibody against | Host | Manufacturer | Dilution |
|---|---|---|---|
| ADAM10 | rabbit | GeneTex | 1:1000 |
| bFGF | rabbit | Santa Cruz Biotechnology | 1:200 |
| ET-1 | rabbit | Abgent | 1:1000 |
| GAPDH | mouse | Proteintech | 1:1000 |
| MMP2 | rabbit | Abgent | 1:1000 |
| MMP9 | mouse | Abgent | 1:500 |
| PAI-1 | rabbit | Proteintech | 1:1000 |
| phospho-Smad1/5 | rabbit | Abgent | 1:1000 |
| (Ser463/465) | | | |
| phospho-Smad3 | rabbit | GeneTex | 1:1000 |
| (Ser423/425) | | | |
| Smad1/5/9 | rabbit | Ameritech Biomedicines | 1:1000 |
| Smad3 | rabbit | GeneTex | 1:1000 |
| Smad4 | rabbit | Assay Biotechnology | 1:1000 |
| TGF-β | rabbit | Abcam | 1:200 |
| TSP-1 | rabbit | GeneTex | 1:1000 |
| VEGF | rabbit | Santa Cruz Biotechnology | 1:200 |
| mouse | goat | Santa Cruz Biotechnology | 1:5000 |
| rabbit | goat | Santa Cruz Biotechnology | 1:5000 |

Antibodies used for western blots, CO-IP and immunostaining studies were validated whenever possible in our laboratory using known positive and negative controls (samples where the antigen is known to be, respectively, present, or absent, either naturally or using knockdown/overexpression strategies, or by treating cells with growth factors that induce/inhibit expression of the targets). When electrophoresis was carried out, band sizes were always checked for the expected molecular weight using molecular weight standards. Different working dilutions and blocking buffers were also evaluated in each case to minimize or even fully remove background staining and to ensure genuine target staining. Whenever possible, we run old stock of antibody (as a positive control) alongside the new stock. Secondary antibody only controls were always performed in each cell type to ensure that secondary antibodies were not binding to nonspecific cellular components, resulting in false positives and/or nonspecific binding.

TABLE 2
Sequences of primers used for RT-qPCR.

| Primer | Sequence | SEQ ID NO |
|---|---|---|
| ALK1-forward | 5′-AGGGCAAACCAGCCATTG-3′ | SEQ ID NO: 1 |
| ALK1-reverse | 5′-GGTTGCTCTTGACCAGCACAT-3′ | SEQ ID NO: 2 |
| ALK2-forward | 5′-GACGTGGAGTATGGCACTATCG-3′ | SEQ ID NO: 3 |
| ALK2-reverse | 5′-CACTCCAACAGTGTAATCTGGCG-3′ | SEQ ID NO: 4 |
| ALK5-forward | 5′-GACAACGTCAGGTTCTGGCTCA-3′ | SEQ ID NO: 5 |
| ALK5-reverse | 5′-CCGCCACTTTCCTCTCCAAACT-3′ | SEQ ID NO: 6 |
| B2M-forward | 5′-CCACTGAAAAAGATGAGTATGCCT-3′ | SEQ ID NO: 7 |
| B2M-reverse | 5′-CCAATCCAAATGCGGCATCTTCA-3′ | SEQ ID NO: 8 |
| BMP4-forward | 5′-AGCATGTCAGGATTAGCCGA-3′ | SEQ ID NO: 9 |
| BMP4-reverse | 5′-TGGAGATGGCACTCAGTTCA-3′ | SEQ ID NO: 10 |
| BMP9-forward | 5′-CATTGTGCGGAGCTTCAGCATG-3′ | SEQ ID NO: 11 |
| BMP9-reverse | 5′-CTGGTGATCTGCTCATGCCTAG-3′ | SEQ ID NO: 12 |
| ID1-forward | 5′-GCCGAGGCGGCATGCGTTC-3′ | SEQ ID NO: 13 |
| ID1-reverse | 5′-TCCCTCAGATCCGGCGAGGC-3′ | SEQ ID NO: 14 |
| HIF-1α-forward | 5′-TATGAGCCAGAAGAACTTTTAGGC-3′ | SEQ ID NO: 15 |
| HIF-1α-reverse | 5′-CACCTCTTTTGGCAAGCATCCTG-3′ | SEQ ID NO: 16 |
| MGP-forward | 5′-CCTCAGCAGAGATGGAGAGCTA-3′ | SEQ ID NO: 17 |
| MGP-reverse | 5′-ATGGCGTAGCGTTCGCAAAGTC-3′ | SEQ ID NO: 18 |
| MMP2-forward | 5′-CCATTTTGATGACGATGAGCTATG-3′ | SEQ ID NO: 19 |
| MMP2-reverse | 5′-GTTGTACTCCTTGCCATTGAACAA-3′ | SEQ ID NO: 20 |
| MMP9-forward | 5′-TTGACAGCGACAAGAAGTGG-3′ | SEQ ID NO: 21 |
| MMP9-reverse | 5′-GCCATTCACGTCGTCCTTAT-3′ | SEQ ID NO: 22 |
| PAI-1-forward | 5′-CTCATCAGCCACTGGAAAGGCA-3′ | SEQ ID NO: 23 |
| PAI-1-reverse | 5′-GACTCGTGAAGTCAGCCTGAAAC-3′ | SEQ ID NO: 24 |
| hsa-miR-18a | 5′-UAAGGUGCAUCUAGUGCAGAUAG-3′ | SEQ ID NO: 25 |
| HIF1A | 5′-UCAUUUUAAAAAAUGCACCUUU-3′ | SEQ ID NO: 26 |

### Methods

### Transfection of Cells

Small interfering RNA transfection was performed as previously described⁵. Briefly, ECs were transfected with small interfering RNA (siRNA) against ID1 or PAI-1 (Thermo Fisher Scientific), or small interfering control (Santa Cruz Biotechnology) constructs using Lipofectamine RNAiMax (Thermo Fisher Scientific) per the manufacturer's instructions. Knockdown of ID1 or PAI-1 was confirmed by quantitative real-time polymerase chain reaction as described⁶ (FIG. 19). The sequences of the forward and reverse primers for ID1 and PAI-1 were included in Table 2.

Plasmid constructs pcDNA3-ALK2^{WT} (Addgene #80870) and pcDNA3-ALK2^{Q207D} (Addgene #80871) were a gift from Aristidis Moustakas⁷. (Meyer B, Schaller C, Frenkel C, Ebeling B, Schramm J. Distributions of local oxygen saturation and its response to changes of mean arterial blood pressure in the cerebral cortex adjacent to arteriovenous malformations. Stroke. 1999; 30(12):2623-2630) 10⁴ AVM-BEC were seeded 24 h before transfection. The plasmid vector containing the 3′ untranslated region (3′UTR) of human BMP4, pRP [Exp]-Puro-CMV>Luciferase:{hBMP4_3′UTR}, was constructed and packaged by VectorBuilder. The vector ID is VB200205-1078zdv, which can be used to retrieve detailed information about the vector on vectorbuilder.com. Transient transfections were performed with Lipofectamine RNAiMax (Thermo Fisher Scientific) according to the manufacturer's instructions. After 4 h, medium was replaced by fresh medium containing microRNA.

### Flow Cytometry Studies

The apoptotic and necrotic cell death was measured by flow cytometry using PI/Annexin V-Alexa Fluor 488 (ThermoFisher Scientific), following the manufacturer's instructions. After 24 h-treatments, cells were harvested, washed with cold PBS, and suspended in Annexin Binding Buffer (ThermoFisher Scientific) at a concentration of 1×10⁶ cells/mL. Following addition of Annexin V and PI, cells were incubated at room temperature for 15 min in the [symbol] dark, and additional 400 μL of 1× binding buffer was added to each sample. Positive controls of cell death were obtained treating cells with 50% ethanol. Ten thousand cells/sample were analyzed using a BD LSRII flow cytometer with the proper compensation controls and the FlowJo_V10 analysis software.

### Mouse Genotyping

Tail tip tissue from the mice was collected at around 14 days of age. Genomic DNA was extracted from the tissue using a lysis buffer consisting of 0.2 mg/mL Proteinase K (VWR) in DirectPCR (Viagen) solution, in accordance with the instructions provided by Viagen. Polymerase chain reaction (PCR) amplification of our MGP sequences was carried out using Accustart II GelTrack PCR SuperMix (VWR). Primer sequences were: SEQ. ID. NO. 027; 028; 029; and 030. The PCR was performed using a Veriti Thermal Cycler (ThermoFisher Scientific), and consisted of incubation at 94° C. for 4 min, followed by 35 cycles of 94° C. for 30 sec, 55° C. for 30 sec and 72° C. for 2 min, and a final incubation of 8 min at 72° C. The amplified fragments in the PCR product were then separated by electrophoresis in 2% agarose gel (Genesee Scientific) with Safe DNA Stain (Genesee Scientific), followed by visualization under UV (iBright CL1000, Invitrogen).

### Mice Inclusion/Exclusion Criteria

Prior inclusion and exclusion criteria for mice used in all in vivo experiments were based on age and genotype. Only new litters with pups at 14 days of age were genotyped for experimentation, and only mice with the desired genotypes at 18-21 days of age were included in the experiments. These timelines were chosen due to the very rapid and severe progression of the AVM disease model. Heterozygous mice that had aged past about 18 months were also excluded from being part of mating pairs as we wanted to optimize the chances of mating and conception.

Sex was not considered a biological variable in this study. Both sexes were used for all genotypes in all the in vivo experiments discussed in this study.

### Animal Randomization Procedures

For most of our in vivo experiments, the groups used were Mgp^{-/-} untreated (Group 1), Mgp^{-/-} scramble microRNA treated (Group 2), Mgp^{-/-} mirR-18a treated (Group 3), Mgp^{-/-} miR-18a and NEO100 treated (Group 4), Mgp^{+/-} (Group 5) and wildtype (Group 6). As new litters of mice pups were born and genotyped, the pups were placed in the various groups based both on the order of which they were born, as well as on their genotypes. For instance, if three Mgp^{-/-} mice were born into three different litters, they would be placed in Groups 1, 2 and 3, respectively. This order of placement ensured that the mice in each group were from different litters and different parents, this method of randomization eliminated any potential intra-litter variance.

Group sizes were determined predominantly based on availability of the different genotypes, ensuring that a statistically significant number of mice was assigned to each group for every in vivo experiment.

### Animal Blinding Procedures

All setup of mating pairs, genotyping and treatment was performed solely by a single researcher, with each mouse tagged by a unique identifier. For imaging, the mice were transported to the imaging core, where they were processed and scanned by a technician who was privy only to the tag numbers and not to which treatment group each mouse belonged to. Furthermore, the data files gathered from imaging were then given to a different researcher for analysis who was told only the tag numbers of each mouse and not the genotype or type of treatment received.

### Computed Tomography (CT)

In vivo ultra-high-resolution computed tomography angiography (CTA) was performed to study changes in neurovascular architecture like methods described previously. (Mukundan S, Jr., Ghaghada K B, Badea C T, et al. A liposomal nanoscale contrast agent for preclinical CT in mice. AJR Am J Roentgenol. 2006; 186(2):300-307) (Starosolski Z, Villamizar C A, Rendon D, et al. Ultra-High-Resolution In vivo Computed Tomography Imaging of Mouse Cerebrovasculature Using a Long Circulating Blood Pool Contrast Agent. Scientific reports. 2015; 5:10178).

Briefly, mice were anesthetized using 3-4% isoflurane, positioned on a CT cradle and maintained at 1-1.5% isoflurane delivered via nose cone. Mice were intravenously injected a long circulating liposomal-iodine blood pool contrast agent (2.2 g/kg body weight) for CTA^{8,9} and scanned on a small animal micro-CT scanner Rigaku CT Lab GX90-1-E. The following scan parameters were used: High Resolution Scan Mode, 70 kVp, 114 uA, 10 mm FOV, 1440 projections, 20 μm voxel size, 14 min scan time.

Biotinylated miR-18a Uptake Analysis

Biotinylated hsa-miR-18a-5p sense and hsa-miR-18a-3p sense probes were acquired from Integrated DNA Technologies (IDT) and reconstituted in RNAse-free water. A scramble sequence of the miR-18a was designed using GenScript®, and the biotinylated probe using this sequence was also acquired from IDT and reconstituted in RNAse-free water. As indicated in FIG. 9, AVM-BECs were transfected with small interfering RNA (siRNA) against Ago-2 (Thermo Fisher Scientific), or small interfering control (Santa Cruz Biotechnology) constructs using Lipofectamine RNAiMax (Invitrogen) per the manufacturer's instructions. Then, media was replaced by fresh media containing the corresponding probes without Lipofectamine, and AVM-BEC were incubated for 30 min at 37° C. in shear flow conditions. When indicated, Ago-2 (0.4 nM, Abcam) was co-administered with the probes as the microRNA carrier and stabilizer¹. Cells were then washed with PBS and fixed with 4% paraformaldehyde (PFA). Avidin/Biotin blocking kit (Vector Laboratories) was used to block unspecific bindings, and cells were incubated with Alexa Fluor 488 streptavidin (1:200, Thermo Fisher Scientific) for 1 h at room temperature to detect the tagged microRNAs. DAPI (Thermo Fisher Scientific) was used to detect cell nuclei. Cells were mounted with Dako Fluorescence Mounting Medium. Pictures were taken in a Zeiss LSM 510 confocal microscope (Carl Zeiss Inc.).

### Treatments

MiR-18a (hsa-miR-18a-5p, 40 nM, Dharmacon) or a scramble microRNA (40 nM, Dharmacon) in nuclease-free water was added with argonaute 2 (Ago-2, 0.4 nM, Abcam) as the microRNA carrier and stabilizer⁹. (Ferreira R, Santos T, Amar A, et al. Argonaute-2 promotes miR-18a entry in human brain endothelial cells. J Am Heart Assoc. 2014; 3(3):e000968). No transfection reagent was used with miR-18a or scramble microRNA treatments. Recombinant human TSP-1 (R&D Systems) stock solution was prepared in PBS and further diluted in culture medium to be used at a final concentration of 1000 ng/mL. Cells were treated during 24 h for mRNA and 48 h for protein expression studies. NEO100 (NeOnc Technologies) stock was prepared in DMSO and used immediately or stored at −20° C.

### Protein Expression Analysis

The levels of angiogenesis-related proteins were analyzed using the Proteome Profiler™ Human Angiogenesis Array kit (R&D Systems) following the manufacturer's instructions. The protocol for western blot was published¹⁰. (Marín-Ramos N I, Alonso D, Ortega-Gutiérrez S, et al. New inhibitors of angiogenesis with antitumor activity in vivo. J Med Chem. 2015; 58(9):3757-3766). Information about antibodies used was included in Table 1. Membranes were visualized and quantified using a C-Digit Blot scanner (Li-Cor) or a iBright CL1000 (Thermo Fisher Scientific).

### Co-Immunoprecipitation

Co-immunoprecipitation (co-IP) was performed using the Thermo Scientific Pierce co-IP kit following the manufacturer's instructions. Briefly, the anti-TSP-1 (Thermo Fisher Scientific) or the PAI-1 (Proteintech) antibodies, correspondingly, were immobilized for 2 h using AminoLink Plus coupling resin. A small portion of the BEC and AVM-BEC lysates was separated to use as input (C+). The resin was then washed and incubated overnight at 4° C. with the remaining part of the BEC and AVM-BEC lysates. Then, the resin was washed, and the proteins eluted using elution buffer. A negative control (C−) received the same concentration of antibody, but the coupling resin was replaced with control agarose resin provided with the IP kit that was not amine-reactive, preventing covalent immobilization of the antibody onto the resin. Protein samples were then analyzed by western blot as described above.

### ELISA

Cells were collected and analyzed for apoptosis, while supernatants were collected and analyzed for necrosis (Cell Death Detection ELISA^{PLUS}), and for PAI-1, VEGF (Quantikine ELISA Kit, R&D Systems) and TGF-β (Proteintech) protein content, following the manufacturers' instructions. Absorbance was measured using a Fluostar Omega microplate reader (BMG Labtech). Data were normalized to kit controls and number of producing cells.

### MiRNA Target Prediction

Studies of miR-18a target genes and binding sites were performed using the following databases: TargetScan version 7.1, microRNA.org and mirDB¹¹. (Wong N, Wang X. miRDB: an online resource for microRNA target prediction and functional annotations. Nucleic Acids Res. 2015; 43(D1): D146-D152). Only predicted targets with good mirSVR score (<−0.1) were considered¹². (Betel D, Koppal A, Agius P, Sander C, Leslie C. Comprehensive modeling of microRNA targets predicts functional non-conserved and non-canonical sites. Genome Biol. 2010; 11(8): R90).

mRNA Expression Analysis

The extraction and analysis of total mRNA and microRNAs were described^{9,13}. (Ferreira R, Santos T, Amar A, et al. Argonaute-2 promotes miR-18a entry in human brain endothelial cells. J Am Heart Assoc. 2014; 3(3):e000968) (Marín-Ramos N I, Jhaveri N, Thein T Z, Fayngor R A, Chen T C, Hofman F M. NEO212, a conjugate of temozolomide and perillyl alcohol, blocks the endothelial-to-mesenchymal transition in tumor-associated brain endothelial cells in glioblastoma. Cancer Lett. 2018; 442:170-180). The Human Notch Signaling Pathway RT² Profiler PCR Array (Qiagen) was used to analyze the expression of 84 genes related to Notch signaling. For mRNA studies, 02-Microglobulin (B2M) was measured for sample normalization. The primer sequences were included in Table 2. For microRNA analysis, RNU44 and U6 snRNA TaqMan microRNA Control Assays were used for normalization of human and mice samples, respectively.

For the experiments regarding the direct binding of miR-18a to BMP4 (information about the plasmid was included in Methods), the luciferase activity of the hBMP4 3′UTR was read in a Varioskan™ LUX multimode microplate reader (Thermo Fisher Scientific).

### Invasion Assays

The chemoinvasion assay was described¹⁴. (Marín-Ramos N I, Thein T Z, Cho H-Y, et al. NEO212 inhibits migration and invasion of glioma stem cells. Mol Cancer Ther. 2018; 17(3):625-637). Pictures were captured using an Eclipse 80i microscope (Nikon), and cells counted with ImageJ Software (NIH).

### Immunostaining

Immunostaining was performed as previously described¹⁵. (Virrey J J, Golden E B, Sivakumar W, et al. Glioma-associated endothelial cells are chemoresistant to temozolomide. J Neurooncol. 2009; 95(1):13-22). Primary antibodies used were rabbit anti-MMP2, mouse anti-MMP9 (1:150, Abgent), rabbit anti-phospho-Smad1/5 (Ser463/465) (1:50, Abgent), rabbit anti-BMP4, rabbit anti-PAI-1 (1:50, Proteintech), rabbit anti-VEGF (1:50, Santa Cruz Biotechnology), rabbit anti-HIF-1α (1:50, Cell Signaling Technology), and rabbit anti-phospho-Smad3 (Ser423/425) (1:50, GeneTex). Secondary antibodies used were biotinylated anti-rabbit and biotinylated anti-mouse (1:300, Vector Laboratories), accordingly. Pictures were taken in an Eclipse 80i microscope (Nikon). Images shown are representative of three-five independent experiments.

### In Vivo Studies

All animal protocols were approved by the USC's Institutional Animal Care and Use Committee (IACUC) and strictly adhered to. MiR-18a was co-administered with Ago-2 (0.0008 nmol/mouse) to increase stability and cellular uptake⁹ (Ferreira R, Santos T, Amar A, et al. Argonaute-2 promotes miR-18a entry in human brain endothelial cells. J Am Heart Assoc. 2014; 3(3):e000968). (FIG. 9). Details about randomization, blinding, group size and inclusion/exclusion criteria have been included in Methods.

For pharmacokinetic measurements, 0.8 nmol/mouse of miR-18a were administered IV or IN to C57BL/6 mice. At the indicated time points, mice were euthanized, and the serum and brains were collected and flash-frozen in liquid nitrogen (n=6 per time-point and condition). MicroRNAs were isolated using the mirVana™ miRNA Isolation Kit and measured by RT-qPCR as described above.

In vivo efficacy studies were performed using 3-week-old Mgp^{+/-} C57BL/6 mice^{16,17} (Yao Y, Jumabay M, Wang A, Boström K I. Matrix Gla protein deficiency causes arteriovenous malformations in mice. J Clin Invest. 2011; 121(8):2993-3004) (Yao Y, Yao J, Radparvar M, et al. Reducing Jagged 1 and 2 levels prevents cerebral arteriovenous malformations in matrix Gla protein deficiency. Proc Nat Acad Sci USA. 2013; 110(47):19071-19076). provided by Dr. Yucheng Yao (UCLA, Los Angeles) and mated to obtain the appropriate genotypes. Only about 10-15% of each litter were born with the Mgp^{-/-} genotype, and among those, only about 45% survived until the end of the treatment and/or the imaging of the brain vasculature, due to the extreme severity of the disease in this mouse model. These animals not only suffer AVMs in the brain, but all major organs are also affected and dysfunctional. Thus, AVM (Mgp^{-/-}) mice might die due to several causes, including stroke, severe lung inflammation that causes respiratory failure—especially when anesthesia is used—, organ failure due to liver and/or spleen malfunction, heart failure, etc., drastically limiting the number of the Mgp^{-/-} mice available for the CTA studies. A total of 41 mice were treated, divided in the following groups based on genotype and treatment: untreated Mgp^{-/-} (n=7), scramble microRNA-treated Mgp^{-/-} (n=6), miR-18a-treated Mgp^{-/-} (n=10), miR-18a+NEO100-treated Mgp^{-/-} (n=3), untreated Mgp^{+/-} (n=8) and untreated Mgp^{+/+} (n=7). Treatment with 0.08 nmol/mouse of miR-18a or scramble microRNA was administered IN daily for 2-weeks. 2.5 μL/nostril were delivered in RNase-free water. Ultra-high-resolution in vivo computed tomography angiography (CTA) was performed in mice for 3D imaging of whole brain vascular architecture¹⁸, (Starosolski Z, Villamizar C A, Rendon D, et al. Ultra-High-Resolution In vivo Computed Tomography Imaging of Mouse Cerebrovasculature Using a Long Circulating Blood Pool Contrast Agent. Scientific reports. 2015; 5:10178) as described in Data. The vascular data was quantified using ImageJ. Regions of interest encompassing the Circle of Willis (CoW) and the azygous of the anterior cerebral artery (AzACA) and its branches were selected in CT images using thick slab maximum intensity projection (MIP) or 3D volume rendering. The images were converted to 8-bit grayscale and thresholded to highlight the vasculature containing pixels. The pixels were analyzed as particles and normalized to results from the wildtype mice (WT, Mgp^{+/+}).

Immediately after imaging, mice were euthanized, and brains were frozen in Clear Frozen Section Compound (VWR) and stored at −80° C. Blood was collected for complete blood count (Antech). The lungs, livers, spleens, femurs, and kidneys were harvested and fixed in 10% formalin. Photographs of the stained sections were captured using an Eclipse 80i microscope (Nikon).

### Statistical Analysis

Statistical analyses were performed using GraphPad Prism 8. Gaussian (normal) distribution was determined using Shapiro-Wilks's normality test and/or the Kolmogorov-Smirnov normality test with Dallal-Wilkinson-Lillie for P value. P<0.05 was considered statistically significant, using unpaired two-tailed t-test, or 1-way or 2-way ANOVA followed by Bonferroni's or Dunnet's multiple comparison test, accordingly. Each patient-derived sample was considered the unit of analysis and accounted for an independent n value, assayed in triplicate. At least three different patient-derived samples were used per experiment. Data were presented as mean±standard error of the mean (SEM). Unless stated otherwise, statistical tests were performed for every experiment shown. When the sets of data that were being compared in each experiment were not significantly different, we indicated so with ns in the graphs or in the corresponding figure legends. When “representative images” are shown, the selected images were those that most accurately represented the average data obtained in all the samples.

### Results

### MiR-18a Normalizes the Levels of VEGF Through PAI-1 in AVM-BEC

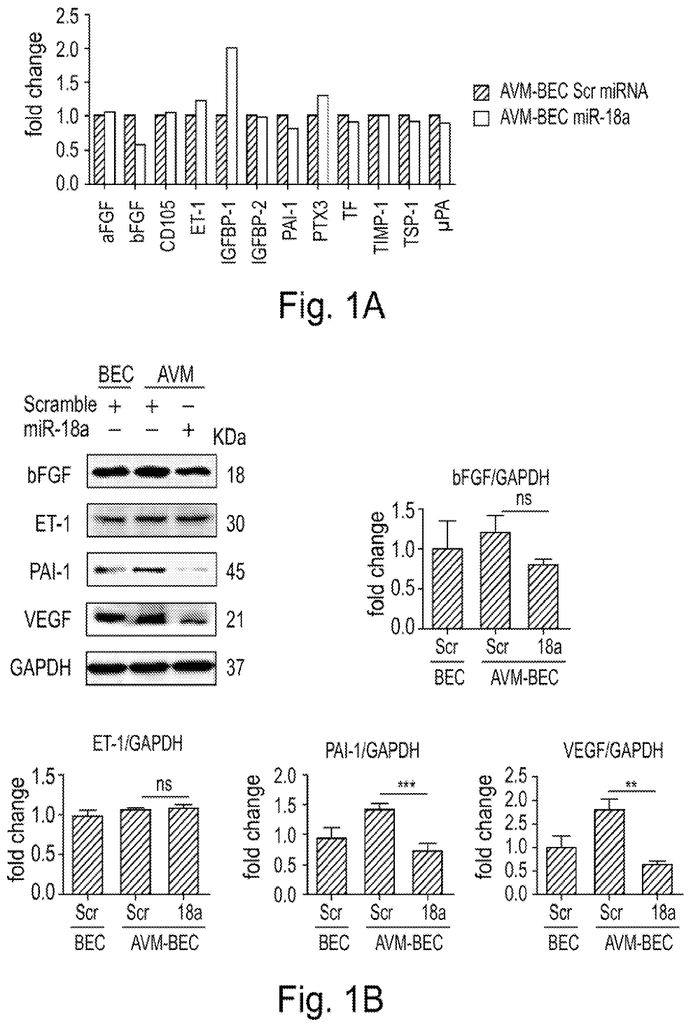

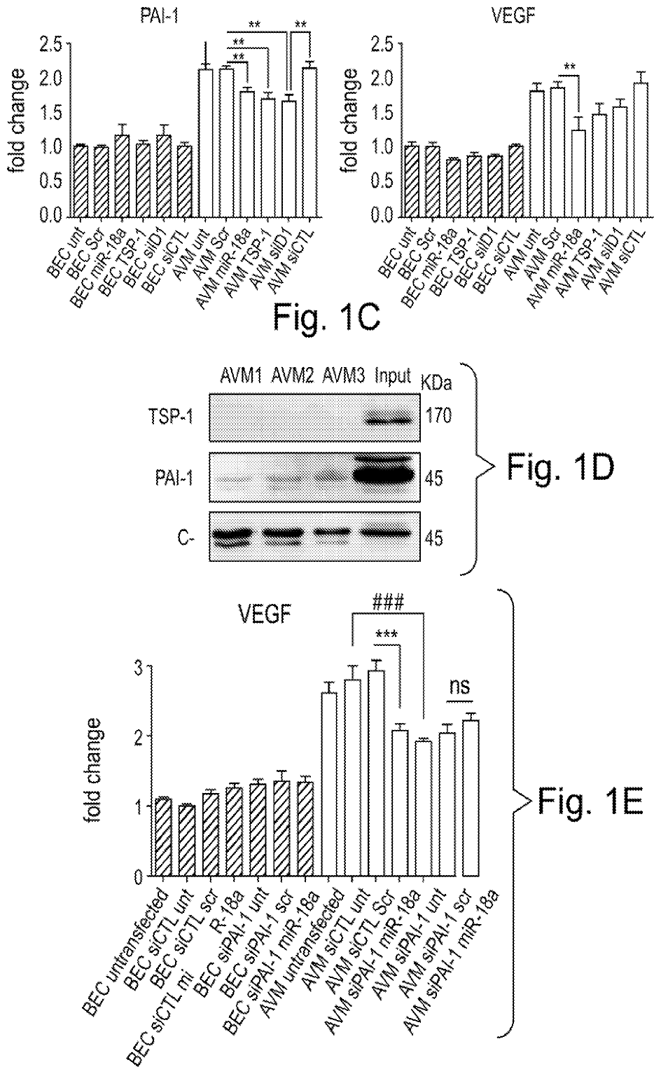

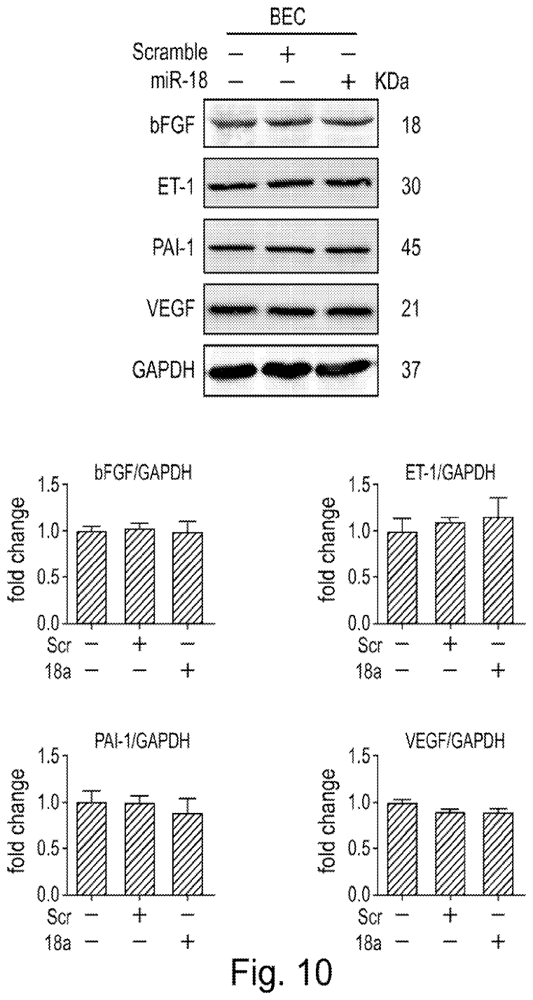

As angiogenesis is a key factor in AVM formation and progression^{4,19}, (Kim H, Su H, Weinsheimer S, Pawlikowska L, Young W L. Brain arteriovenous malformation pathogenesis: A response-to-injury paradigm. Acta Neurochir Suppl. 2011; 111:83-92) (Lu L, Bischoff J, Mulliken J B, Bielenberg D R, Fishman S J, Greene A K. Progression of arteriovenous malformation: Possible role of vasculogenesis. Plast Reconstr Surg. 2011; 128(4):260e-269e) we examined the changes in the levels of 55 angiogenesis-related proteins in AVM-BECs upon miR-18a treatment. Among those whose levels were altered by miR-18a in AVM-BECs, bFGF levels showed a 2-fold decrease compared to scramble miR-treated AVM-BEC, while IGFBP-1 was 2-fold upregulated. MiR-18a also caused a >20% upregulation of endothelin-1 (ET-1) and pentraxin 3 (PTX3); and a 20% downregulation of PAI-1 (FIG. 1A). Previous results from our laboratory showed that treatment with miR-18a induces TSP-1 release by AVM-BECs, as evidence by ELISA quantification of cell supernatants⁶. (Ferreira R, Santos T, Amar A, et al. MicroRNA-18a improves human cerebral arteriovenous malformation endothelial cell function. Stroke. 2014; 45(1):293-297). However, we did not observe any relevant differences in TSP-1 protein levels obtained from AVM-BEC lysates (FIG. 1A). These data suggest that miR-18a is affecting the release of TSP-1, rather than the levels of TSP-1 protein production. Among the 55 proteins included in the proteome array, those factors that were found to be expressed in the AVM-BECs, and especially, those whose levels were altered by miR-18a towards a less proangiogenic phenotype were further analyzed by western blot. For these protein expression studies, control BECs were included, and experiments were performed with at least three different patient-derived cells to allow for statistical analysis. Some of the factors expressed in the preliminary screening were not expressed in all samples, or the results were too variable amongst the different patient samples to establish a pattern (data not shown). Only bFGF, ET-1 and PAI-1 were expressed in all samples and showed a constant pattern when comparing BECs and AVM-BECs, as well as scramble microRNA versus miR-18a. When compared to BEC, only PAI-1 levels were consistently higher in AVM-BEC and decreased with miR-18a in all patient-derived AVM-BEC. This expression pattern was very similar to that of VEGF (FIG. 1B), whose relative overexpression is a prominent feature in AVM⁴. The levels of these proangiogenic factors remained unaltered between untreated BECs, scramble microRNA-treated BECs and miR-18a-treated BECs (FIG. 10).

It has been described that PAI-1 induces VEGF expression and release^{20,21}, (Gillespie E, Leeman S E, Watts L A, et al. Plasminogen activator inhibitor-1 is increased in colonic epithelial cells from patients with colitis-associated cancer. J Crohns Colitis. 2013; 7(5):403-411) (Hjortland G O, Lillehammer T, Somme S, et al. Plasminogen activator inhibitor-1 increases the expression of VEGF in human glioma cells. Exp Cell Res. 2004; 294(1):130-139) and PAI-1 and TSP-1 levels are inversely related in renal cell carcinoma²². (Zubac D P, Wentzel-Larsen T, Seidal T, Bostad L. Type 1 plasminogen activator inhibitor (PAI-1) in clear cell renal cell carcinoma (CCRCC) and its impact on angiogenesis, progression and patient survival after radical nephrectomy. BMC Urology. 2010; 10(1):20). We then studied whether PAI-1 could have a role in the inverse correlation between the low levels of secreted TSP-1 and the high levels of secreted VEGF in AVM-BECs. To test this hypothesis, we treated AVM-BEC with miR-18a or TSP-1 recombinant protein. To confirm the mechanistic importance of TSP-1 in regulating PAI-1 expression, we performed a small interfering RNA-mediated knock-down (siID1) of the inhibitor of DNA binding 1 (ID1), a known repressor of TSP-1 that is upregulated in AVM-BEC^{5,23}. (Stapleton C J, Armstrong D L, Zidovetzki R, Liu C Y, Giannotta S L, Hofman F M. Thrombospondin-1 modulates the angiogenic phenotype of human cerebral arteriovenous malformation endothelial cells. Neurosurgery. 2011; 68(5):1342-1353) (Volpert O V, Pili R, Sikder H A, et al. Id1 regulates angiogenesis through transcriptional repression of thrombospondin-1. Cancer Cell. 2002; 2(6):473-483). These controls were added based on previous data from our laboratory showing that miR-18a treatment induces TSP-1 release and inhibits ID1 expression in AVM-BECs^{5,6}. (Stapleton C J, Armstrong D L, Zidovetzki R, Liu C Y, Giannotta S L, Hofman F M. Thrombospondin-1 modulates the angiogenic phenotype of human cerebral arteriovenous malformation endothelial cells. Neurosurgery. 2011; 68(5):1342-1353) (Ferreira R, Santos T, Amar A, et al. MicroRNA-18a improves human cerebral arteriovenous malformation endothelial cell function. Stroke. 2014; 45(1):293-297). Treatments with miR-18a and TSP-1, as well as ID1 knock-down, decreased the levels of secreted PAI-1 and VEGF in AVM-BEC supernatants, without affecting control BEC. The decrease in VEGF, although present under all three conditions, only reached significance with miR-18a treatment. Completely untreated BECs and AVM-BECs were included as controls for microRNA treatments. No significant (P>0.9999) differences were observed between the untreated cells and the corresponding scramble microRNA-treated cells. Additionally, a small interfering control construct (siCTL) was used as a control of transfection using Lipofectamine for siID1 transfections. No significant (P>0.9999) differences were observed between the untreated cells and the corresponding siCTL-treated cells (FIG. 1C).

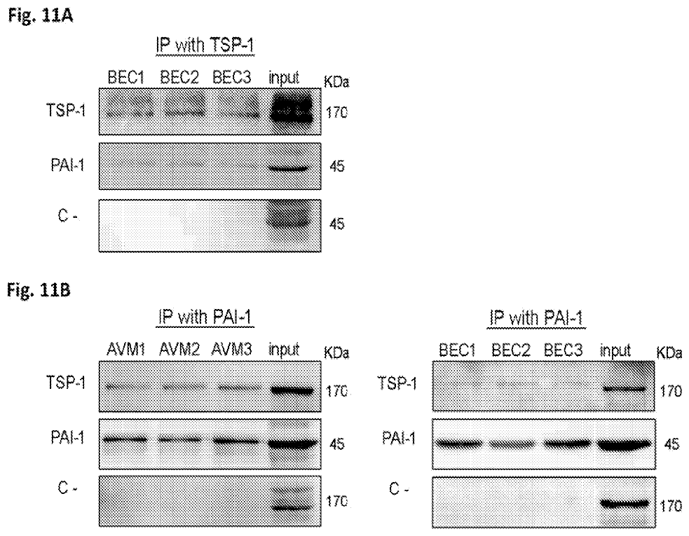

We wanted to comprehend whether TSP-1, which had already been shown to have a role in the pathobiology of AVM⁵ as well as in the AVM-BEC normalization driven by miR-18a⁶, (Ferreira R, Santos T, Amar A, et al. MicroRNA-18a improves human cerebral arteriovenous malformation endothelial cell function. Stroke. 2014; 45(1):293-297) was directly interacting with PAI-1 or the potential link between both factors was indirect. To determine whether TSP-1 directly binds to PAI-1 or additional factors are involved, we performed a co-IP assay of both proteins in three patient-derived AVM-BEC samples. Controls with BEC samples, as well as controls of reverse co-IP in both BEC and AVM-BEC samples were also performed (FIG. 11). We detected PAI-1 in all samples immunoprecipitated with TSP-1 antibody, which suggests that both proteins directly bind to each other (FIG. 1D). Taken together, these results indicate that miR-18a is specifically suppressing PAI-1 and VEGF levels in AVM-BECs, while not affecting their levels in control BECs.

Finally, to establish a direct role of PAI-1 in VEGF regulation, we performed a small interfering RNA-mediated knock-down of PAI-1 (siPAI-1) and repeated the ELISA analysis of VEGF in additional AVM-BEC and BEC patient samples. A small interfering control construct (siCTL) was used as a control of transfection using Lipofectamine for siPA-1 transfections. Untreated BECs and AVM-BECs were included as controls for microRNA treatments and showed no significant differences (P>0.9999) when compared to the corresponding scramble microRNA-treated cells. Treatment with miR-18a decreased the levels of secreted VEGF in AVM-BEC supernatants, without affecting control BEC, only when cells were transfected with siCTL. PAI-1 silencing caused a comparable decrease in AVM-BEC, and in this case (siPAI-1) the effects of miR-18a in the decrease of the secreted VEGF levels were lost (FIG. 1E). These data suggest that miR-18a might be decreasing VEGF levels through PAI-1 regulation.

### MiR-18a Downregulates BMP4 Signaling in AVM-BEC

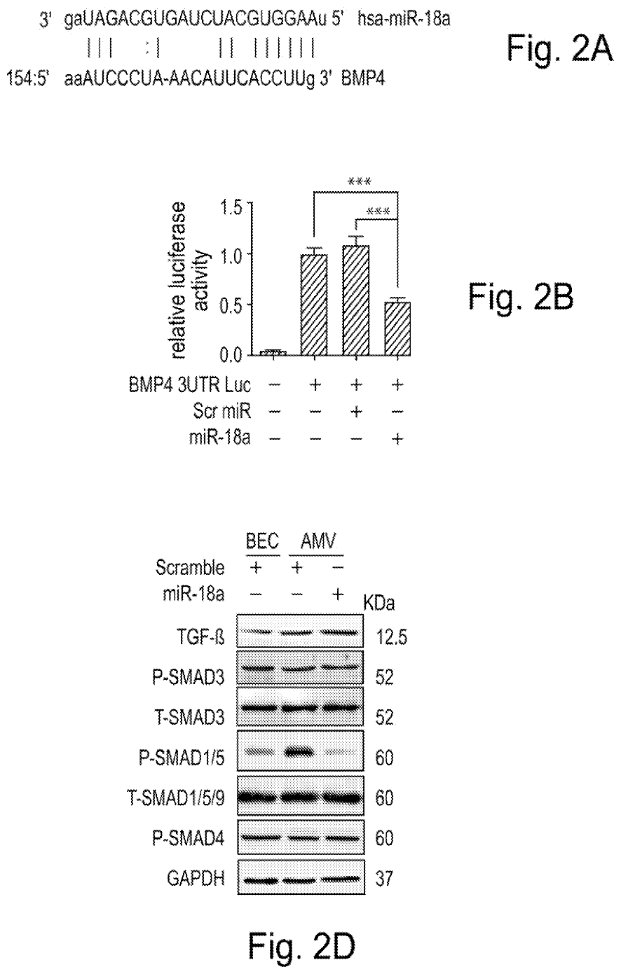

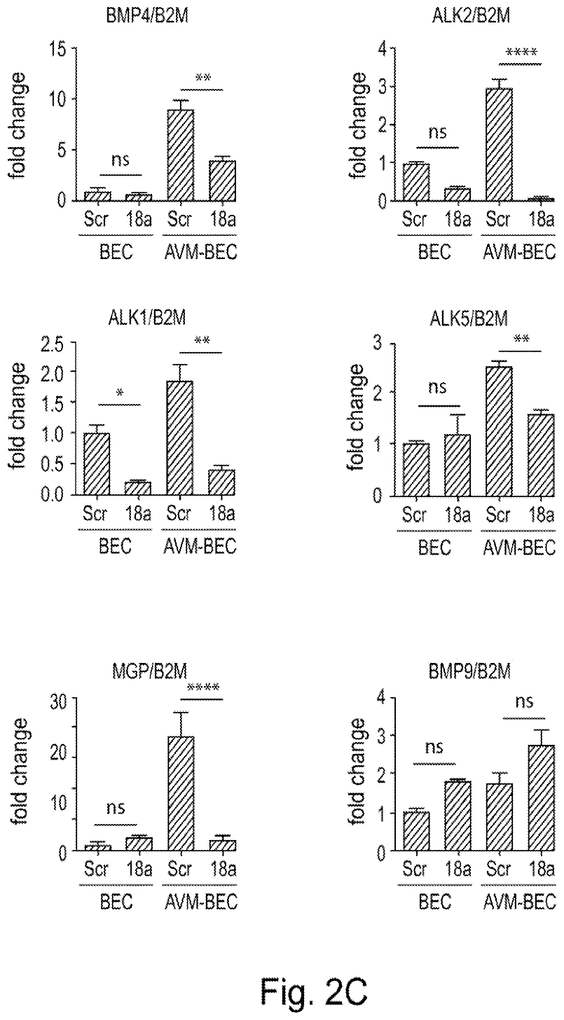

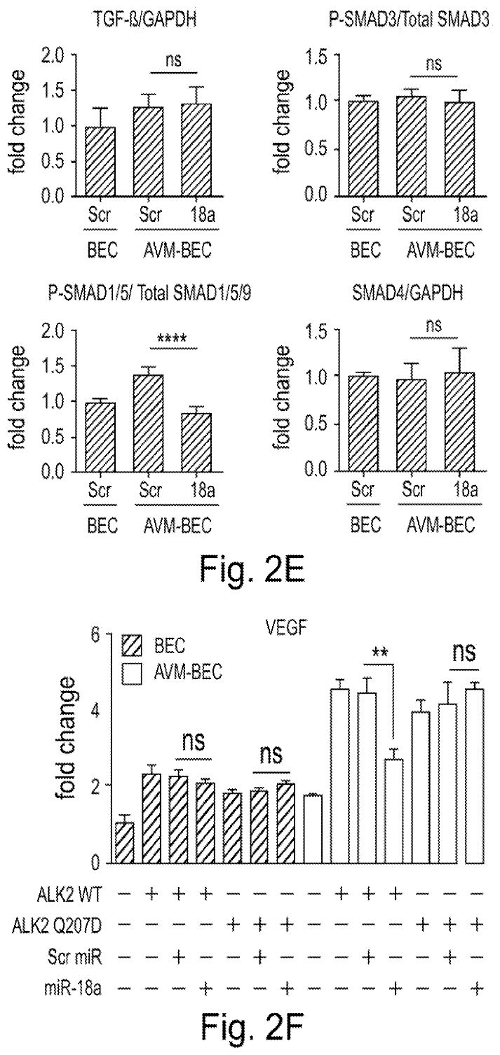

MiR-18a should decrease ID1 expression⁶ (Ferreira R, Santos T, Amar A, et al. MicroRNA-18a improves human cerebral arteriovenous malformation endothelial cell function. Stroke. 2014; 45(1):293-297). by regulating an upstream factor, as it has no complementary sequence alignment with ID1. To search for potential miR-18 target genes, we used TargetScan, microRNA.org and mirDB databases. We then cross-matched the results obtained from the software analysis with those from the literature review regarding factors involved in angiogenesis and AVM. We chose BMP4 and HIF-1α as potential candidates, since they are both factors related to both AVM and angiogenesis^{17,24-33}, (Yao Y, Yao J, Radparvar M, et al. Reducing Jagged 1 and 2 levels prevents cerebral arteriovenous malformations in matrix Gla protein deficiency. Proc Natl Acad Sci USA. 2013; 110(47):19071-19076; Yao Y, Zebboudj A F, Shao E, Perez M, Boström K. Regulation of bone morphogenetic protein-4 by matrix GLA protein in vascular endothelial cells involves activin-like kinase receptor 1. J Biol Chem. 2006; 281(45):33921-33930; Ng I, Tan W L, Ng P Y, Lim J. Hypoxia inducible factor-1alpha and expression of vascular endothelial growth factor and its receptors in cerebral arteriovenous malformations. J Clin Neurosci. 2005; 12(7):794-799; Rothhammer T, Bataille F, Spruss T, Eissner G, Bosserhoff A K. Functional implication of BMP4 expression on angiogenesis in malignant melanoma. Oncogene. 2007; 26(28):4158-4170; Rezzola S, Di Somma M, Corsini M, et al. VEGFR2 activation mediates the pro-angiogenic activity of BMP4. Angiogenesis. 2019; 22(4):521-533; Takagi Y, Kikuta K, Moriwaki T, et al. Expression of thioredoxin-1 and hypoxia inducible factor-1alpha in cerebral arteriovenous malformations: Possible role of redox regulatory factor in neoangiogenic property. Surgical neurology international. 2011; 2:61; Li L, Pan H, Wang H, et al. Interplay between VEGF and Nrf2 regulates angiogenesis due to intracranial venous hypertension. Scientific reports. 2016; 6(1):37338; Lim C S, Kiriakidis S, Sandison A, Paleolog E M, Davies A H. Hypoxia-inducible factor pathway and diseases of the vascular wall. Journal of Vascular Surgery. 2013; 58(1):219-230; Shi Y-H, Fang W-G. Hypoxia-inducible factor-1 in tumour angiogenesis. World J Gastroenterol. 2004; 10(8):1082-1087; Zimna A, Kurpisz M. Hypoxia-Inducible Factor-1 in Physiological and Pathophysiological Angiogenesis: Applications and Therapies. Biomed Res Int. 2015; 2015:549412-549412; and Hashimoto T, Shibasaki F. Hypoxia-Inducible Factor as an Angiogenic Master Switch. Frontiers in Pediatrics. 2015; 3(33)) as well as direct targets of miR-18a (sequence alignments in FIGS. 2A,3A). HIF-1α has already been empirically proven to be a target of miR-18a³⁴⁻³⁶. (Montoya M M, Maul J, Singh P B, et al. A distinct inhibitory function for miR-18a in Th17 cell differentiation. J Immunol. 2017; 199(2):559-569; Krutilina R, Sun W, Sethuraman A, et al. MicroRNA-18a inhibits hypoxia-inducible factor 1α activity and lung metastasis in basal breast cancers. Breast Cancer Research. 2014; 16(4):R78; and Wu F, Huang W, Wang X. microRNA-18a regulates gastric carcinoma cell apoptosis and invasion by suppressing hypoxia-inducible factor-1α expression. Exp Ther Med. 2015; 10(2):717-722 Nevertheless, although the hBMP4 3′UTR sequence aligns with that of miR-18a (http://www.microrna.org/microma/getMrna.do?gene=652&utr=15959&organism=9606 #) and therefore BMP4 is a predicted target of miR-18a, to our knowledge, the studies showing the interaction between BMP4 and miR-18a are merely indirect, involving other TGF-β/BMP signaling-related factors, and/or the whole miRNA-17-92 cluster to which miR-18a belongs³⁷⁻⁴⁰. (Wang J, Greene S B, Bonilla-Claudio M, et al. Bmp signaling regulates myocardial differentiation from cardiac progenitors through a MicroRNA-mediated mechanism. Developmental cell. 2010; 19(6):903-912; Li L, Shi J-Y, Zhu G-Q, Shi B. MiR-17-92 cluster regulates cell proliferation and collagen synthesis by targeting TGFB pathway in mouse palatal mesenchymal cells. Journal of Cellular Biochemistry. 2012; 113(4):1235-1244; Luo T, Cui S, Bian C, Yu X. Crosstalk between TGF-β/Smad3 and BMP/BMPR2 signaling pathways via miR-17-92 cluster in carotid artery restenosis. Molecular and Cellular Biochemistry. 2014; 389(1):169-176; and Schwentner R, Herrero-Martin D, Kauer M O, et al. The role of miR-17-92 in the miRegulatory landscape of Ewing sarcoma. Oncotarget. 2017; 8(7):10980-10993). Hence, we transfected ECs with a plasmid containing the 3′-UTR region of BMP4 together with a luciferase reporter (BMP4 3′UTR Luc). As shown in FIG. 2B, the BMP4-3′-UTR-Luc activity in the ECs was significantly (P<0.001) decreased with miR-18a treatment in comparison to those untreated or treated with scramble microRNA, providing further evidence that BMP4 is a direct target of miR-18a.

We first studied the potential effects of miR-18a on the BMP4 pathway in AVM. Yao et a. had previously identified a regulatory pathway induced by BMP4 in ECs, where BMP4 interacts with ALK2 to stimulate expression of ALK1, which in turn induces the expression of MGP and ALK5. They also showed that ALK2 expression is stimulated by enhanced BMP4 signaling, while the induced ALK1 receptor is stimulated by BMP9, and ALK5 is stimulated by TGF-β1 to induce VEGF expression^{16,24,41-43}. (Yao Y, Jumabay M, Wang A, Boström K I. Matrix Gla protein deficiency causes arteriovenous malformations in mice. J Clin Invest. 2011; 121(8):2993-3004; Yao Y, Zebboudj A F, Shao E, Perez M, Boström K. Regulation of bone morphogenetic protein-4 by matrix GLA protein in vascular endothelial cells involves activin-like kinase receptor 1. J Biol Chem. 2006; 281(45):33921-33930; Shao E S, Lin L, Yao Y, Bostrom K I. Expression of vascular endothelial growth factor is coordinately regulated by the activin-like kinase receptors 1 and 5 in endothelial cells. Blood. 2009; 114(10):2197-2206; Yao Y, Shao E S, Jumabay M, Shahbazian A, Ji S, Bostrom K I. High-density lipoproteins affect endothelial BMP-signaling by modulating expression of the activin-like kinase receptor 1 and 2. Arteriosclerosis, thrombosis, and vascular biology. 2008; 28(12):2266-2274; and Yao Y, Shahbazian A, Bostrom K I. Proline and gamma-carboxylated glutamate residues in matrix Gla protein are critical for binding of bone morphogenetic protein-4. Circulation research. 2008; 102(9):1065-1074 Furthermore, they proved that the deficiency in matrix GLA protein (MGP), an antagonist of BMP4 signaling, causes AVM in mice by impairing the BMP4/ALK2/ALK1/ALK5/VEGF pathway^{16,24}. (Yao Y, Jumabay M, Wang A, Boström KI. Matrix Gla protein deficiency causes arteriovenous malformations in mice. J Clin Invest. 2011; 121(8):2993-3004; Yao Y, Zebboudj A F, Shao E, Perez M, Boström K. Regulation of bone morphogenetic protein-4 by matrix GLA protein in vascular endothelial cells involves activin-like kinase receptor 1. J Biol Chem. 2006; 281(45):33921-33930). Hence, we analyzed the effects of miR-18a in all these factors involved in this regulatory pathway leading to AVM. Using RT-qPCR, we observed that the basal levels of BMP4, ALK2/1/5 and MGP were higher in AVM-BEC than in BEC, and treatment with miR-18a normalized their expression in AVM-BEC. No significant differences (P>0.05) were observed in BEC upon miR-18a treatment, except that ALK1 levels were decreased (P=0.0482). We also studied the effects of miR-18a on BMP9 expression, which has been related to AVM in HHT patients¹⁶. (Yao Y, Jumabay M, Wang A, Boström KI. Matrix Gla protein deficiency causes arteriovenous malformations in mice. J Clin Invest. 2011; 121(8):2993-3004). No significant differences (P>0.05) were observed with miR-18a in our patient-derived non-HHT related AVM-BEC (FIG. 2C).

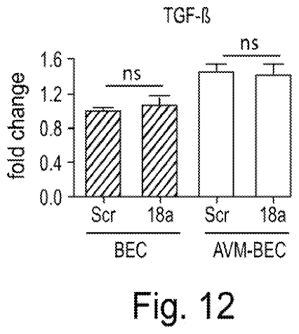

The influence of TGF-β and TGF-β signaling-related genes on AVM pathogenesis has been thoroughly studied^{4,24}. (Kim H, Su H, Weinsheimer S, Pawlikowska L, Young W L. Brain arteriovenous malformation pathogenesis: A response-to-injury paradigm. Acta Neurochir Suppl. 2011; 111:83-92) (Yao Y, Zebboudj A F, Shao E, Perez M, Boström K. Regulation of bone morphogenetic protein-4 by matrix GLA protein in vascular endothelial cells involves activin-like kinase receptor 1. J Biol Chem. 2006; 281(45):33921-33930). To determine whether miR-18a regulates TGF-β signaling, we analyzed the protein levels of mature TGF-β, and the phosphorylation levels of its downstream effector Smad3, together with the downstream effectors of BMP4, Smad1/5. Once phosphorylated, Smad1/3/5 form heteromeric complexes with Smad4, subsequently binding to the DNA and regulating gene expression⁴⁴. (Beets K, Staring M W, Criem N, et al. BMP-SMAD signalling output is highly regionalized in cardiovascular and lymphatic endothelial networks. BMC Dev Biol. 2016; 16(1):34). Since Smad4 has been described as a direct target of miR-18a^{34,45}, (Montoya M M, Maul J, Singh P B, et al. A distinct inhibitory function for miR-18a in Th17 cell differentiation. J Immunol. 2017; 199(2):559-569) (Krutilina R, Sun W, Sethuraman A, et al. MicroRNA-18a inhibits hypoxia-inducible factor 1α activity and lung metastasis in basal breast cancers. Breast Cancer Research. 2014; 16(4):R78) we also analyzed its protein levels. The levels of TGF-β were higher in AVM-BEC than in BEC but were not significantly changed (P>0.9999) with miR-18a, as measured by western blot (FIG. 2D-E) and ELISA (FIG. 12). Neither the phosphorylation levels of Smad3 nor the total levels of Smad4 were altered by miR-18a (P>0.9999). However, the phosphorylation levels of Smad1/5 were higher in AVM-BEC than in BEC, and significantly (P<0.0001) decreased by miR-18a (FIG. 2D-E), confirming its effects on BMP4. To establish the mechanism of action of miR-18a through the BMP4/ALK2 pathway, we transfected the EC with a constitutively active ALK2 (ALK2^{Q207D}) and compared them with cells transfected with wild-type ALK2 (ALK2^{WT}). Constitutively active ALK2 does not require BMP4 binding for activity and should not be sensitive to miR-18a. As expected, transfection of both BEC and AVM-BEC with ALK2 increased VEGF secretion. No significant changes (P>0.9999) were observed with miR-18a treatment in BEC. However, in AVM-BEC transfected with constitutively active ALK2^{Q207D}, miR-18a was unable to decrease VEGF levels (P>0.9999) as it did in ALK2^{WT}-transfected AVM-BEC (P=0.0030) (FIG. 2F).

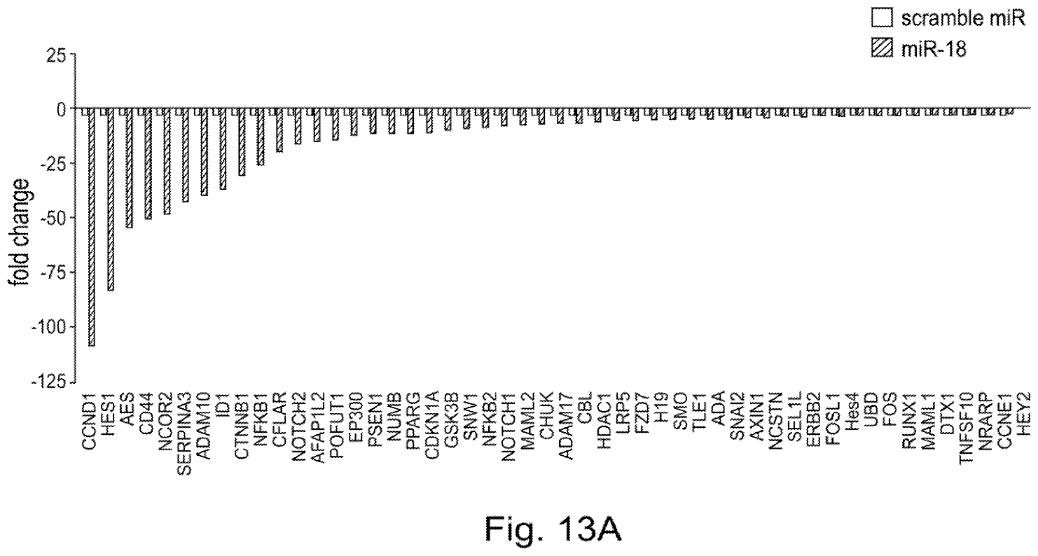

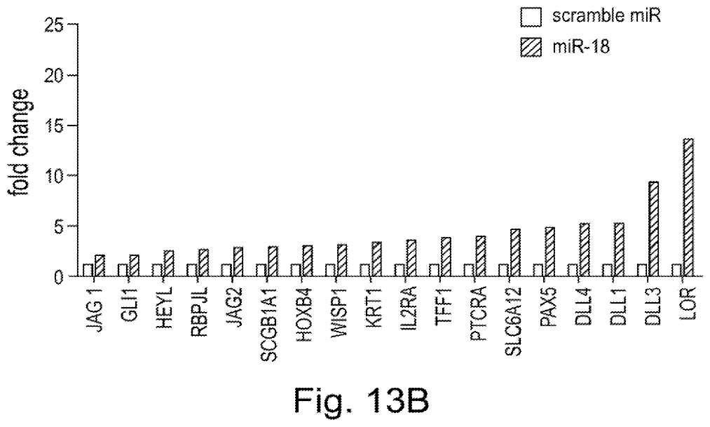

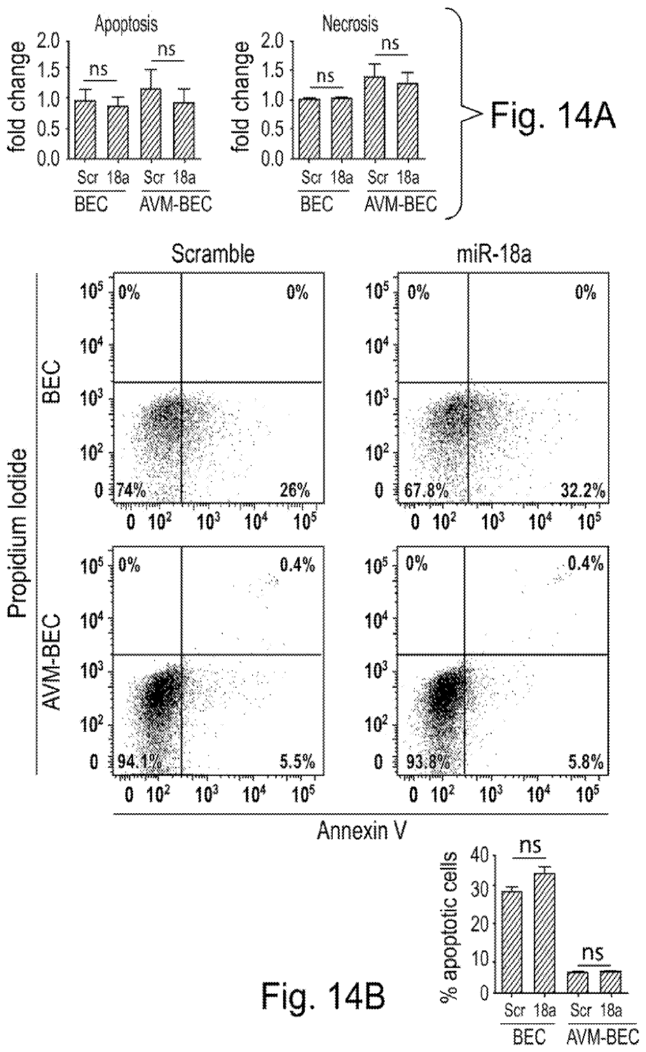

ALK1 enhances Notch signaling in human brain microvascular EC, causing brain AVM in vivo¹⁷. (Yao Y, Yao J, Radparvar M, et al. Reducing Jagged 1 and 2 levels prevents cerebral arteriovenous malformations in matrix Gla protein deficiency. Proc Natl Acad Sci USA. 2013; 110(47):19071-19076). Since miR-18a decreases ALK1 expression, we studied its effects on 84 Notch-related genes using a PCR array. 68 genes were affected by miR-18a (fold change>2), including many related to apoptosis, among which 50 genes were downregulated. These results, together with ELISA and flow cytometry analysis of cell death, were included in FIGS. 13-14. Overall, these data indicate that miR-18a directly targets BMP4, without affecting BMP9 or TGF-β signaling.

### MiR-18a Blocks HIF-1α Signaling in Normoxia

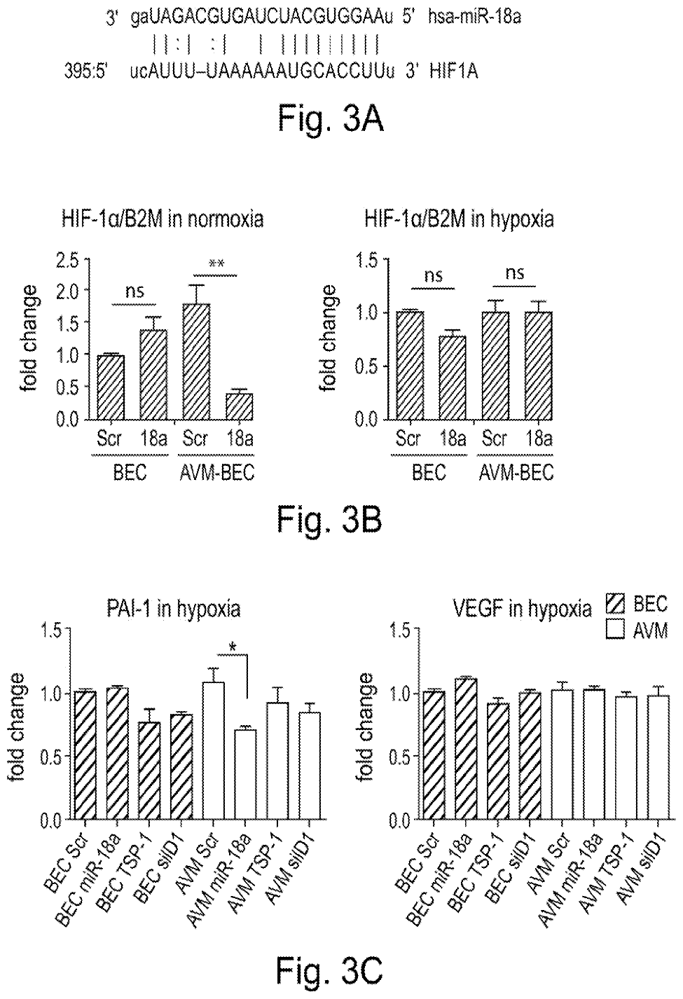

The distribution of oxygen and nutrients in the cortex surrounding AVM is almost identical to the normal cortex, and hypoxia is not common⁷. (Meyer B, Schaller C, Frenkel C, Ebeling B, Schramm J. Distributions of local oxygen saturation and its response to changes of mean arterial blood pressure in the cerebral cortex adjacent to arteriovenous malformations. Stroke. 1999; 30(12):2623-2630). However, HIF-1α is expressed in human cerebral AVM, and its expression correlates with that of VEGF²⁵. (Ng I, Tan W L, Ng P Y, Lim J. Hypoxia inducible factor-1alpha and expression of vascular endothelial growth factor and its receptors in cerebral arteriovenous malformations. J Clin Neurosci. 2005; 12(7):794-799). The direct interaction between miR-18a and HIF-1α has been thoroughly demonstrated in the literature^{34-36,46} (Montoya M M, Maul J, Singh P B, et al. A distinct inhibitory function for miR-18a in Th17 cell differentiation. J Immunol. 2017; 199(2):559-569; Krutilina R, Sun W, Sethuraman A, et al. MicroRNA-18a inhibits hypoxia-inducible factor 1α activity and lung metastasis in basal breast cancers. Breast Cancer Research. 2014; 16(4):R78; Wu F, Huang W, Wang X. microRNA-18a regulates gastric carcinoma cell apoptosis and invasion by suppressing hypoxia-inducible factor-1α expression. Exp Ther Med. 2015; 10(2):717-722; and Han F, Wu Y, Jiang W. MicroRNA-18a Decreases Choroidal Endothelial Cell Proliferation and Migration by Inhibiting HIF1A Expression. Med Sci Monit. 2015; 21:1642-1647) (FIG. 3A). We examined the effects of miR-18a on HIF-1α expression under normoxic and hypoxic conditions in our patient-derived BEC and AVM-BEC samples. The basal levels of HIF-1α were higher in AVM-BEC than in control BEC only when cells were cultured in normoxia. MiR-18a significantly (P=0.0025) decreased HIF-1α expression in AVM-BEC in normoxia, without affecting BEC (P>0.9999). Under hypoxia, the expression levels of HIF-1α remained unaltered in both cases (FIG. 3B).

HIF-1α plays a central role in oxygen homeostasis by inducing the expression of several genes, including VEGF and PAI-1⁴⁷. (Kietzmann T, Samoylenko A, Roth U, Jungermann K. Hypoxia-inducible factor-1 and hypoxia response elements mediate the induction of plasminogen activator inhibitor-1 gene expression by insulin in primary rat hepatocytes. Blood. 2003; 101(3):907-914). We had demonstrated the inhibitory effects of miR-18a in VEGF and PAI-1 secretion in normoxia (FIG. 1C). As PAI-1 and VEGF production is triggered by hypoxia, we performed these experiments in a 3% oxygen-atmosphere. Contrary to what was observed in normoxia, in hypoxia the basal levels of both factors were similar in BEC and AVM-BEC. This is likely due to an increase in their levels in BEC under hypoxia, not present in AVM-BEC where the normoxic levels are already elevated. The reduction of PAI-1 secretion caused by miR-18a was still observed (P=0.0378). However, neither treatment with TSP-1 (P=0.3917), nor ID1 knock-down (P=0.1701) were able to significantly decrease the levels of PAI-1. The levels of VEGF were not affected by any of the treatments under hypoxic conditions (P>0.9999) (FIG. 3C).

### MiR-18a Decreases AVM-BEC Invasion Capacity

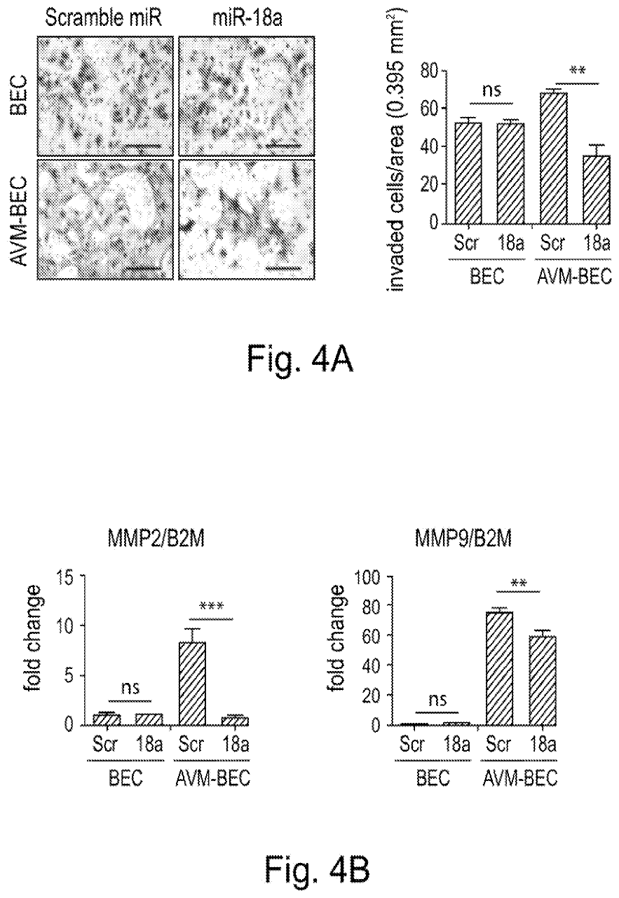

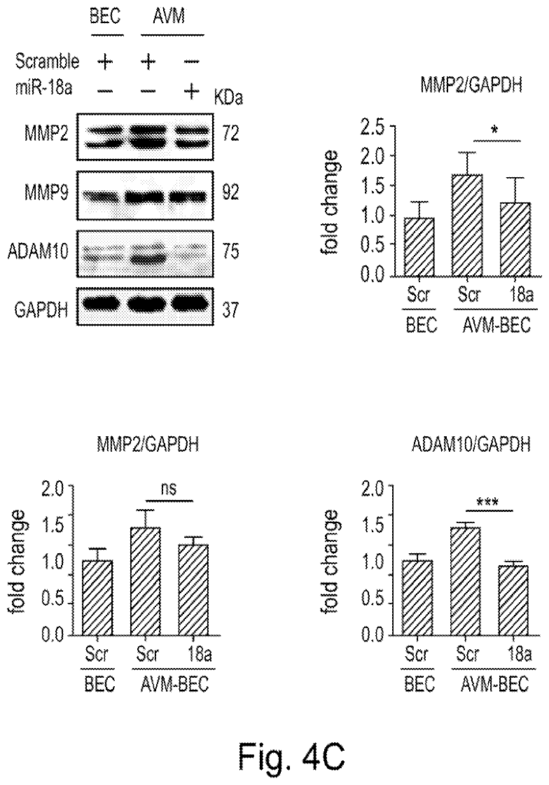

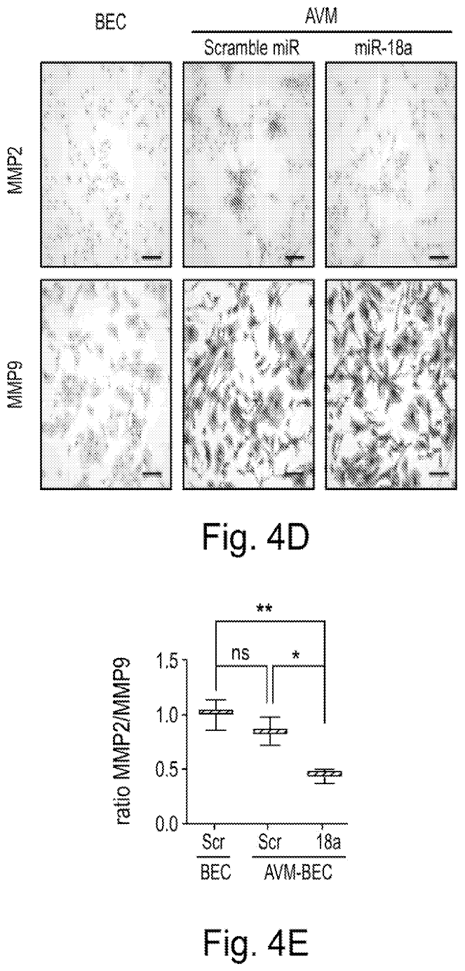

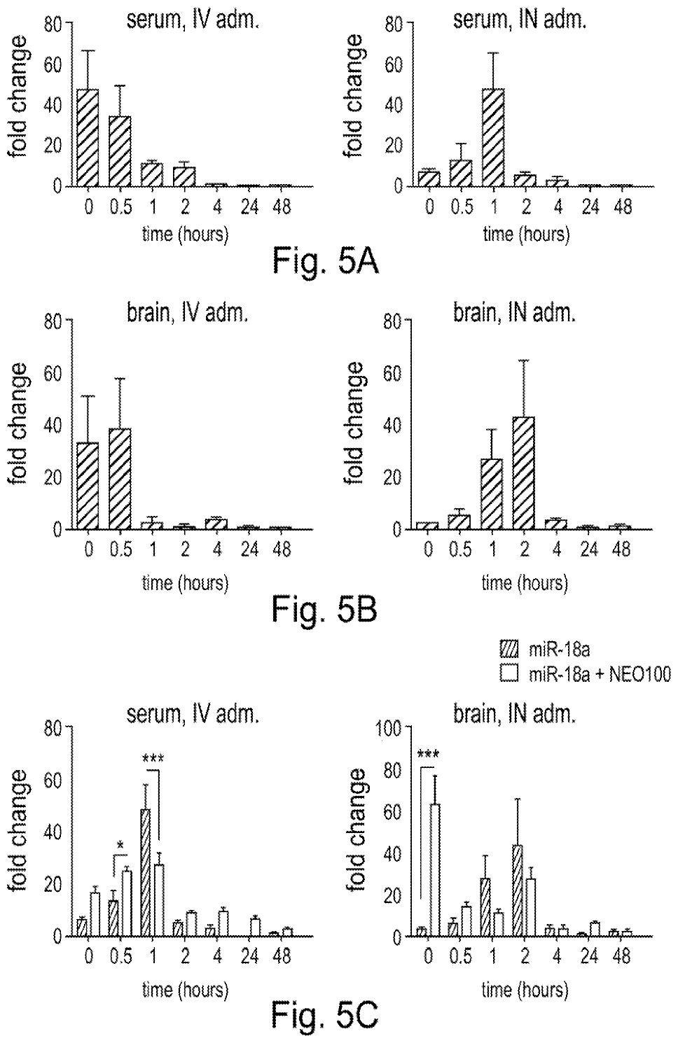

Our previous studies demonstrated that AVM-BEC migrate faster than BEC, and that treatment with TSP-1 decreases their migration rate⁵. (Stapleton C J, Armstrong D L, Zidovetzki R, Liu C Y, Giannotta S L, Hofman F M. Thrombospondin-1 modulates the angiogenic phenotype of human cerebral arteriovenous malformation endothelial cells. Neurosurgery. 2011; 68(5):1342-1353). To test whether miR-18a showed a comparable effect, we performed invasion assays, which involve cell migration and degradation of extracellular matrix (ECM), using a Matrigel-coated Boyden chamber. Treatment with miR-18a decreased AVM-BEC invasiveness, without affecting control BEC (FIG. 4A).

Matrix metalloproteinases (MMPs) are essential enzymes in ECM degradation and angiogenesis regulation⁴⁸. (Kunz P, Sahr H, Lehner B, Fischer C, Seebach E, Fellenberg J. Elevated ratio of MMP2/MMP9 activity is associated with poor response to chemotherapy in osteosarcoma. BMC cancer. 2016; 16:223). We analyzed the levels of MMP2 and MMP9, commonly upregulated in AVM^{49,50}, (Xu M, Xu H, Qin Z, Zhang J, Yang X, Xu F. Increased expression of angiogenic factors in cultured human brain arteriovenous malformation endothelial cells. Cell Biochem Biophys. 2014; 70(1):443-447) (Hashimoto T, Wen G, Lawton M T, et al. Abnormal expression of matrix metalloproteinases and tissue inhibitors of metalloproteinases in brain arteriovenous malformations. Stroke. 2003; 34(4):925-931) as well as of ADAM10, a disintegrin-metalloproteinase that promotes cell migration and invasion^{51,52}, (Schelter F, Kobuch J, Moss M L, et al. A disintegrin and metalloproteinase-10 (ADAM-10) mediates DN30 antibody-induced shedding of the Met surface receptor. J Biol Chem. 2010; 285(34):26335-26340) (Li D, Xiao, Z., Wang, G., & Song, X. Knockdown of ADAM10 inhibits migration and invasion of fibroblast-like synoviocytes in rheumatoid arthritis. Mol Med Rep. 2015; 12:5517-5523) whose mRNA expression was downregulated by miR-18a (FIG. 13). The basal expression levels of MMP2 and MMP9 were considerably higher in AVM-BEC and normalized with miR-18a (FIG. 4B). Western blot analysis showed higher MMP2 and ADAM10 protein levels in AVM-BEC than in BEC, which were significantly decreased by miR-18a (P=0.0132 and P<0.001, respectively). However, the protein levels of MMP9 in AVM-BEC were not significantly (P=0.9409) affected by miR-18a (FIG. 4C). To discern the role of the MMP proteins in miR-18a effects on cell invasion, we performed some immunostaining assays of MMP2 and MMP9. For MMP2, we could observe that the expression of MMP2 correlated with cells showing high rate of proliferation, suggesting that there is an aggressive subpopulation of endothelial cells within the AVM nidus. The results obtained with MMP9 immunostaining were comparable to those obtained with the western blot analysis, with higher expression levels in AVM-BEC than in control BEC, which did not decrease in the presence of miR-18a (FIG. 4C-D). A reduction in MMP2 activity while MMP9 remains unaltered lowers the ratio MMP2/MMP9 (FIG. 4E), which has been suggested as a potential marker of prognosis in osteosarcoma⁴⁸. (Kunz P, Sahr H, Lehner B, Fischer C, Seebach E, Fellenberg J. Elevated ratio of MMP2/MMP9 activity is associated with poor response to chemotherapy in osteosarcoma. BMC cancer. 2016; 16:223).

In Vivo Pharmacokinetic Studies of miR-18a

To determine the optimal dosage and administration route of miR-18a, we performed pharmacokinetic studies using RT-qPCR analysis. MiR-18a was administered IV or IN to C57BL/6 mice. After the indicated time-points, mice were euthanized, and serum and brains were collected. MiR-18a levels were approximately 40 times higher in mice treated with miR-18a as compared to scramble microRNA-treated mice. Following IV administration, the highest levels of miR-18a in serum and brains were detected earlier than with IN administration, which in contrast led to longer retention times (FIG. 5A-B). These data demonstrate that miR-18a reaches the brain and can be administered IN, a minimally invasive drug delivery system.

Our previous studies showed that NEO100 increases the brain delivery of drugs following IN administration^{53,54}. (Marín-Ramos N I, Pérez-Hernández M, Anson T, et al. NEO100 targets glioma stem cells and inhibits motility through the calpain-1/RhoA pathway. J Neurosurgery. 2019: DOI: 10.3171/2019.3175.JNS19798) (Wang W, Swenson S, Cho H Y, Hofman F M, Schonthal A H, Chen T C. Efficient brain targeting and therapeutic intracranial activity of bortezomib through intranasal co-delivery with NEO100 in rodent glioblastoma models. J Neurosurg. 2019; doi: 10.3171/2018.11.JNS181161:1-9). Co-administration of miR-18a with 0.3% NEO100 prolonged the stability of miR-18a in serum (time-points: 2 h to 48 h) and increased the amount of microRNA in the brain (from approximately 40 times the basal levels of miR-18a to approximately 60 times when co-administered with NEO100), reducing the time required to reach the peak levels from 2 h to 5 min after administration (FIG. 5C).

### MiR-18a Shows Therapeutic Efficacy In Vivo in a Mouse Model of AVM

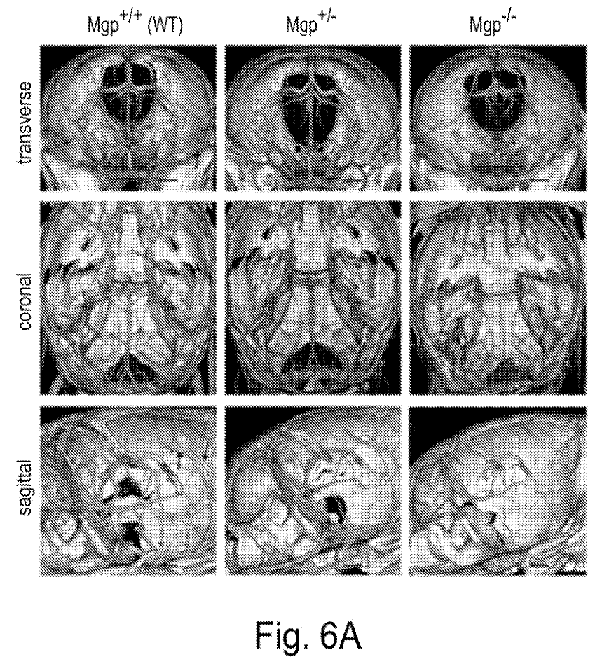

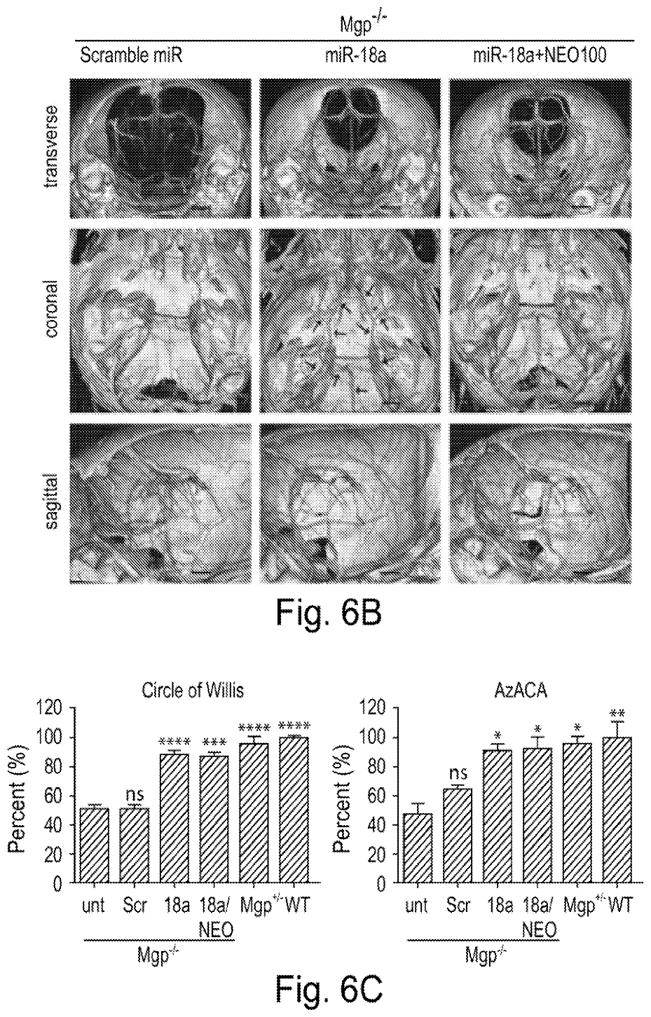

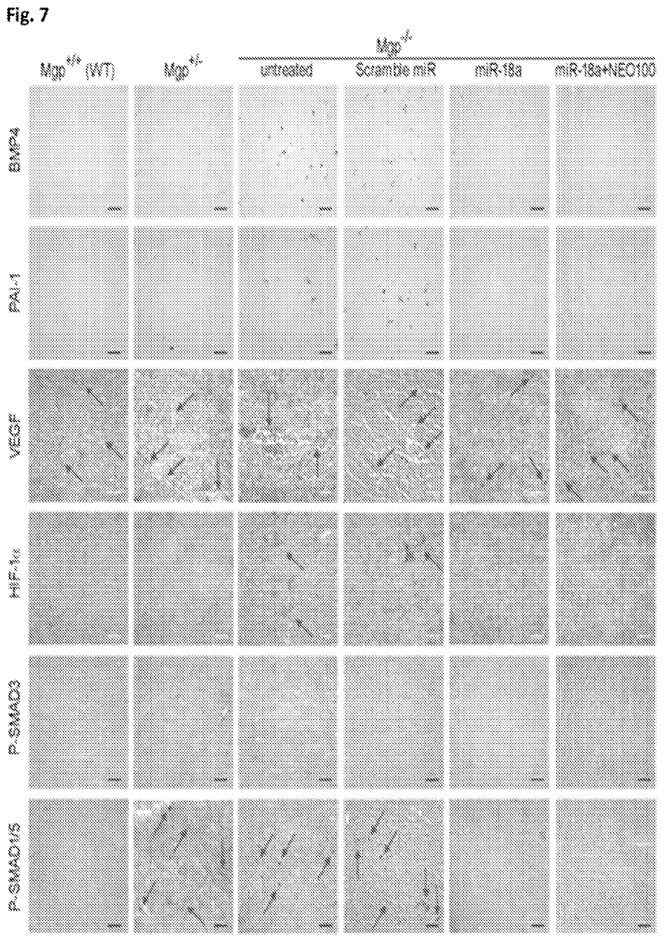

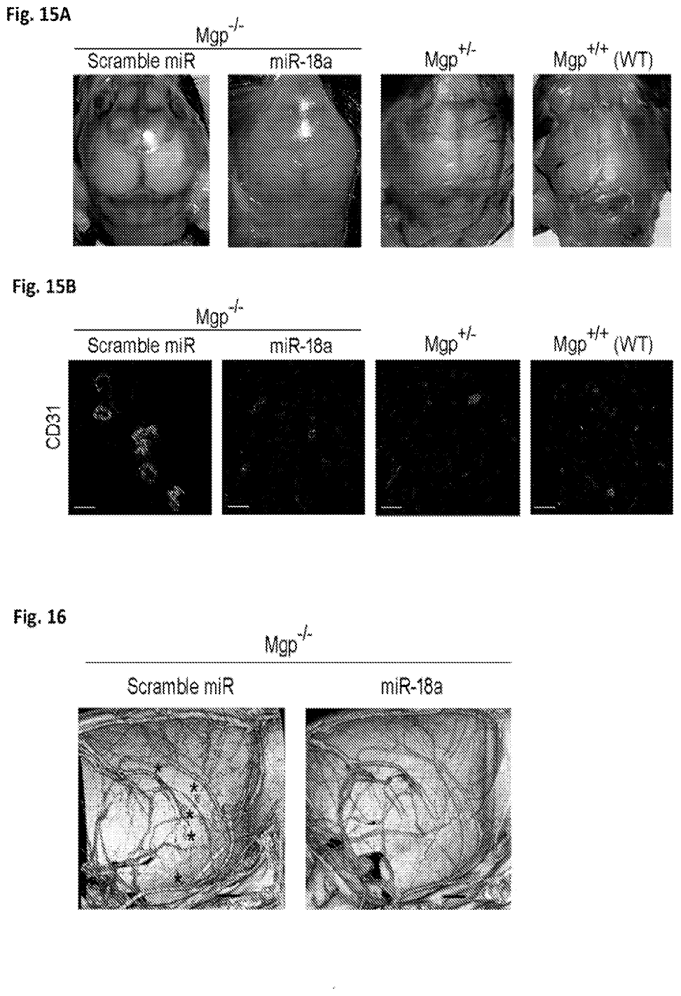

To determine whether miR-18a could block AVM in vivo, we used an Mgp^{-/-} mouse model that develops brain AVM in 100% of the animals^{16,17,55}. (Yao Y, Jumabay M, Wang A, Boström K I. Matrix Gla protein deficiency causes arteriovenous malformations in mice. J Clin Invest. 2011; 121(8):2993-3004; Yao Y, Yao J, Radparvar M, et al. Reducing Jagged 1 and 2 levels prevents cerebral arteriovenous malformations in matrix Gla protein deficiency. Proc Natl Acad Sci USA. 2013; 110(47):19071-19076; and Nielsen C M, Huang L, Murphy P A, Lawton M T, Wang R A. Mouse models of cerebral arteriovenous malformation. Stroke. 2016; 47(1):293-300). Mice co-treated IN with Ago-2 and either miR-18a or scramble microRNA for 2-weeks, underwent ultra-high-resolution CTA for 3D interrogation of whole brain vasculature. Treatment of the AVM Mgp^{-/-} mouse model with miR-18a reduced signs of fresh hemorrhage and presence of abnormally enlarged blood vessels in the brain (FIG. 15). CTA revealed abnormal and reduced neurovasculature, and direct connections between arteries and veins characteristic of AVM niduses in the Mgp^{-/-} mice compared to Mgp^{+/+} (wild type, WT), with heterozygous Mgp^{+/-} showing intermediate characteristics (FIG. 6). Aside from the occurrence of niduses that are consistent with the AVM disease model, Mgp^{-/-} mice showed highly tortuous and strained vasculature throughout the brain, which were especially obvious in the CTA images of anatomical landmarks such as the Circle of Willis (CoW) and azygous of the anterior cerebral artery (AzACA). The CoW of untreated and scramble microRNA-treated Mgp^{-/-} mice was disrupted and incomplete, likely due to the poor cranial circulation. Furthermore, the CoW of Mgp^{-/-} (AVM) mice showed poor branching with high distortion of vessel structures and sometime even missing Superior Cerebral Artery (SCA), Posterior Cerebral Artery (PCA) or Anterior Cerebral Artery (ACA). Based on our quantitative analysis of CTA data, the CoW of untreated and scramble microRNA-treated Mgp^{-/-} mice presented only 48-51% the vessel density compared to that of healthy wildtype (Mgp^{+/+}) (FIG. 6C). Treatment with miR-18a (with and without NEO100), however, significantly improved the CoW vascular density in Mgp^{-/-} mice to approximately 88-89% relative to healthy wildtype mice (FIG. 6C, P<0.0001 and P=0.0002, respectively). Similarly, the AzACA and its branches of untreated and scramble microRNA-treated Mgp^{-/-} mice exhibited the major branches, but the more intricate micro-vasculature appeared to be absent. In general, the AzACA and its branches of Mgp^{-/-} mice exhibited low vascular density (48-65% relative to healthy wildtype mice), missing or poorly developed branches and high distortion of vessel structure. Conversely, miR-18a (with and without NEO100) appeared to normalize the vasculature (FIG. 6C, P=0.0237 and P=0.0186, respectively), as more branching and micro-vasculature were observed by CTA post-treatment. The AzACA vasculature of knockout mice treated with miR-18a exhibited approximately 92-94% vessel density compared to healthy wildtype mice. Multiple focal spots of highly dense signal in the untreated and scramble miR-treated Mgp^{-/-} mice suggested regions of high vascular leakage and calcifications, both findings typical of AVM formations^{56,57}. Although miR-18a normalized brain vasculature and leakage (FIG. 6B and FIG. 16), it showed no effect on the calcifications found in carotids of all Mgp^{-/-} mice (FIG. 17).

To confirm whether the signaling pathways implicated in vitro (FIGS. 1-3) were also being affected in vivo, we performed immunohistochemistry (IHC) studies of brain sections from these mice (FIG. 7). The expression of BMP4, PAI-1, VEGF, and HIF-1α, as well as the activation (phosphorylation) of Smad1/5, were higher in brains from untreated and scramble microRNA-treated Mgp^{-/-} (AVM) mice, compared to Mgp^{+/+} (WT) and/or Mgp^{+/-} mice. Treatment with miR-18a, with or without NEO100, decreased the expression of BMP4, PAI-1, VEGF, and HIF-1α, as well as the activation of the BMP4-downstream effectors Smad1/5. No differences were observed in the phosphorylation levels of the TGF-β-downstream effector Smad3 amongst the different conditions. Immunostaining of the brains for VEGF was consistent with the results obtained with the endothelial marker CD31 (FIG. 15) and with previous results from the literature¹⁷. Mgp^{-/-} mice showed a lack of normal vasculature in the absence of treatment or with scramble microRNA treatment, where the only blood vessels detected were abnormally enlarged and tortuous, with exceptionally low presence of normal-sized blood vessels. MiR-18a normalized the appearance of the brain vasculature.

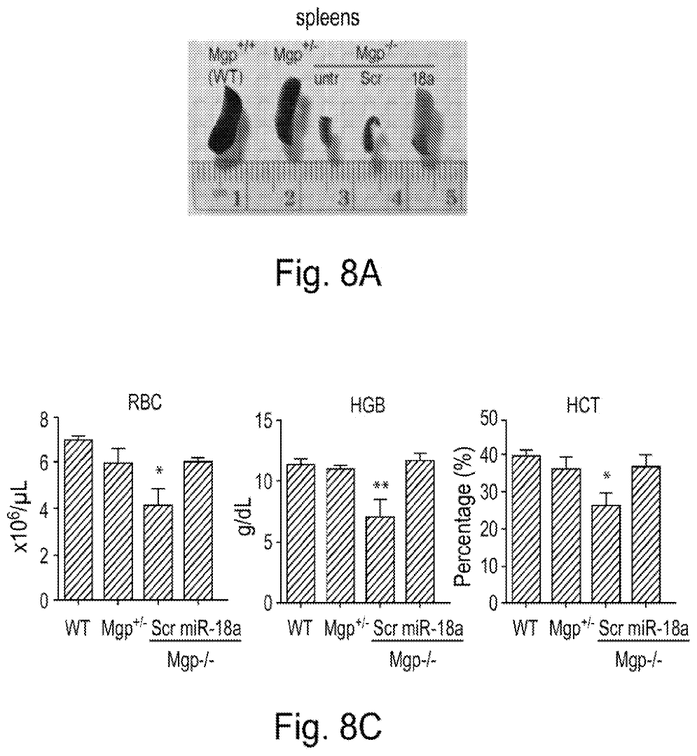

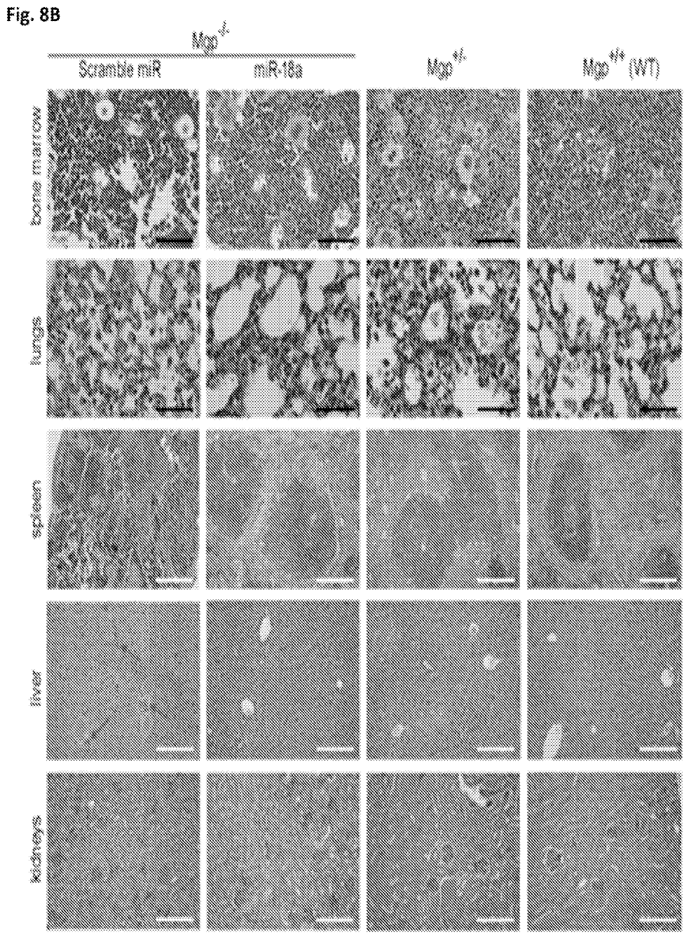

We then examined the effects of miR-18a in the bone marrow, lungs, spleen, liver, and kidneys (FIG. 8). Macroscopically, the spleens of Mgp^{+/+}, Mgp^{-/+} and miR-18a-treated Mgp^{-/-} were comparable, while those of untreated and scramble microRNA-treated Mgp^{-/-} were equally underdeveloped (FIG. 8A). No relevant differences were observed among the different conditions in the rest of the organs, except for an overall smaller size in the untreated and scramble microRNA-treated Mgp^{-/-} mice. Microscopically, the bone marrows of scramble microRNA-treated Mgp^{-/-} mice showed high levels of adipose cells and very few, if any, megakaryocytes. This phenotype was normalized by treatment with miR-18a (FIG. 8B). The lack of bone marrow precursors correlated with lower blood levels of red blood cells (RBC), hemoglobin (HGB) and hematocrit (HCT) in the scramble microRNA-treated Mgp^{-/-} mice. These levels were normalized with miR-18a treatment (FIG. 8C). The blood tests also showed in the scramble miR-treated mice a marked polychromasia, a disorder demonstrating abnormally high numbers of immature red blood cells in the bloodstream because of premature release of cells from the bone marrow. The polychromasia was moderate in the Mgp^{+/-} and minimal or not present in the Mgp^{+/+} and miR-18a-treated Mgp^{-/-} mice. No significant differences were observed in the other blood tests (P>0.05, data not shown).

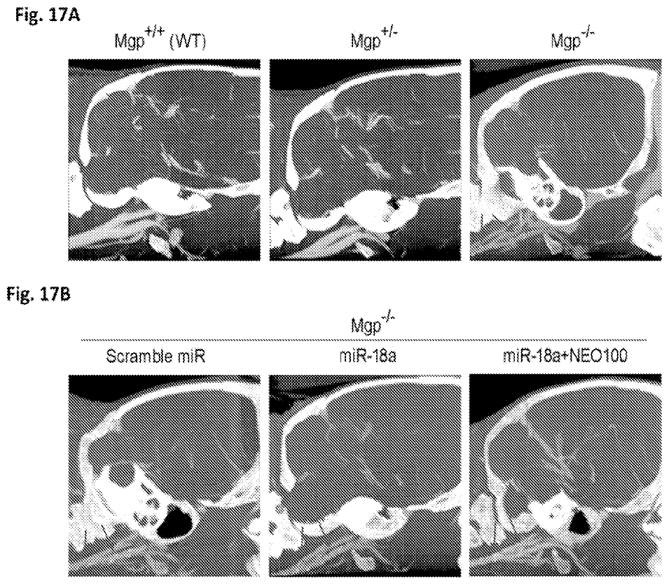

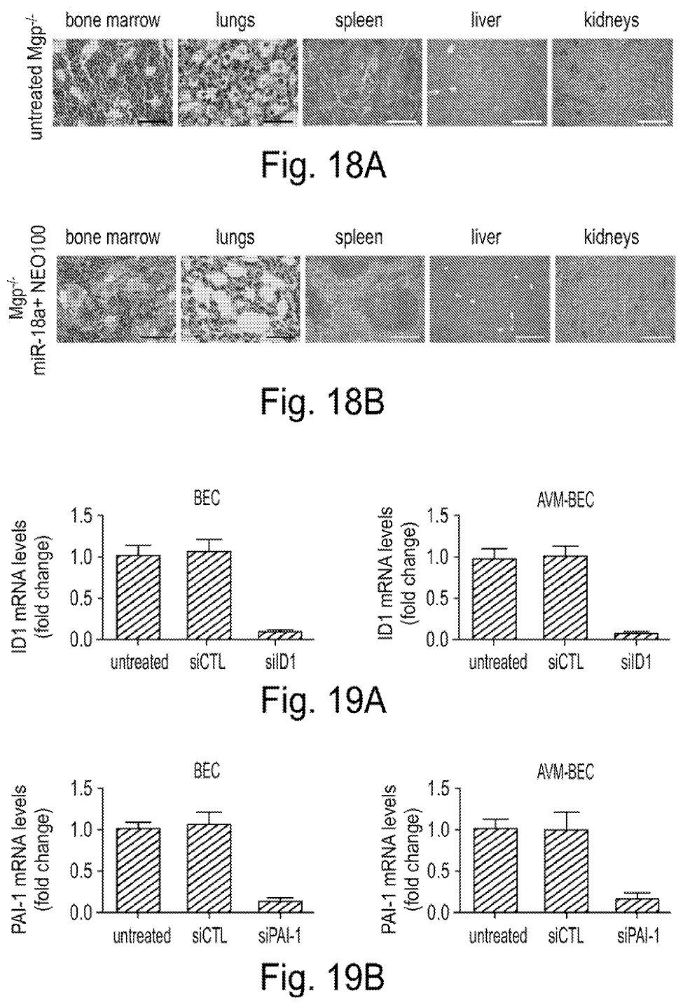

The lungs of the scramble microRNA-treated Mgp^{-/-} had macrophages in their alveoli, indicating lung inflammation, not present with miR-18a treatment. The spleens of the scramble microRNA-treated mice showed absence of white matter, as evidenced by the lack of germinal center structures. The livers of the scramble microRNA-treated Mgp^{-/-} mice showed no structured hepatic lobules/bile ducts, abnormal vasculature, and inflammatory cell infiltration. No differences were observed between the untreated and the scramble microRNA-treated Mgp^{-/-} mice, or between the miR-18a-treated mice with and without NEO100; neither in the kidneys from different conditions (FIG. 8B and FIG. 18).

The scope of the present invention is not limited by what has been specifically shown and described hereinabove. Those skilled in the art will recognize that there are suitable alternatives to the depicted examples of materials, configurations, constructions, and dimensions. Numerous references, including patents and various publications, are cited, and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications, and other implementations of what is described herein will occur to those of ordinary skill in the art without departing from the spirit and scope of the invention. While certain embodiments of the present invention have been shown and described, it will be obvious to those skilled in the art that changes, and modifications may be made without departing from the spirit and scope of the invention. The matter set forth in the foregoing description is offered by way of illustration only and not as a limitation.

### REFERENCES

1. Braksick S A, Fugate J E. Management of brain arteriovenous malformations. Curr Treat Options Neurol. 2015; 17(7):32.
2. Dupuis-Girod S, Ginon I, Saurin J, et al. Bevacizumab in patients with hereditary hemorrhagic telangiectasia and severe hepatic vascular malformations and high cardiac output. JAMA. 2012; 307(9):948-955.
3. Lebrin F, Srun S, Raymond K, et al. Thalidomide stimulates vessel maturation and reduces epistaxis in individuals with hereditary hemorrhagic telangiectasia. Nat Med. 2010; 16:420-428.
4. Kim H, Su H, Weinsheimer S, Pawlikowska L, Young W L. Brain arteriovenous malformation pathogenesis: A response-to-injury paradigm. Acta Neurochir Suppl. 2011; 111:83-92.
5. Stapleton C J, Armstrong D L, Zidovetzki R, Liu C Y, Giannotta S L, Hofman F M. Thrombospondin-1 modulates the angiogenic phenotype of human cerebral arteriovenous malformation endothelial cells. Neurosurgery. 2011; 68(5):1342-1353.
6. Ferreira R, Santos T, Amar A, et al. MicroRNA-18a improves human cerebral arteriovenous malformation endothelial cell function. Stroke. 2014; 45(1):293-297.
7. Meyer B, Schaller C, Frenkel C, Ebeling B, Schramm J. Distributions of local oxygen saturation and its response to changes of mean arterial blood pressure in the cerebral cortex adjacent to arteriovenous malformations. Stroke. 1999; 30(12):2623-2630.
8. Taguchi A, Yanagisawa K, Tanaka M, et al. Identification of hypoxia-inducible factor-1α as a novel target for mir-17-92 microrna cluster. Cancer Res. 2008; 68(14):5540-5545.
9. Ferreira R, Santos T, Amar A, et al. Argonaute-2 promotes miR-18a entry in human brain endothelial cells. J Am Heart Assoc. 2014; 3(3):e000968.
10. Marín-Ramos N I, Alonso D, Ortega-Gutiérrez S, et al. New inhibitors of angiogenesis with antitumor activity in vivo. J Med Chem. 2015; 58(9):3757-3766.
11. Wong N, Wang X. miRDB: an online resource for microRNA target prediction and functional annotations. Nucleic Acids Res. 2015; 43(D1):D146-D152.
12. Betel D, Koppal A, Agius P, Sander C, Leslie C. Comprehensive modeling of microRNA targets predicts functional non-conserved and non-canonical sites. Genome Biol. 2010; 11(8):R90.
13. Marín-Ramos N I, Jhaveri N, Thein T Z, Fayngor R A, Chen T C, Hofman F M. NEO212, a conjugate of temozolomide and perillyl alcohol, blocks the endothelial-to-mesenchymal transition in tumor-associated brain endothelial cells in glioblastoma. Cancer Lett. 2018; 442:170-180.
14. Marín-Ramos N I, Thein T Z, Cho H-Y, et al. NEO212 inhibits migration and invasion of glioma stem cells. Mol Cancer Ther. 2018; 17(3):625-637.
15. Virrey J J, Golden E B, Sivakumar W, et al. Glioma-associated endothelial cells are chemoresistant to temozolomide. J Neurooncol. 2009; 95(1):13-22.
16. Yao Y, Jumabay M, Wang A, Boström K I. Matrix Gla protein deficiency causes arteriovenous malformations in mice. J Clin Invest. 2011; 121(8):2993-3004.
17. Yao Y, Yao J, Radparvar M, et al. Reducing Jagged 1 and 2 levels prevents cerebral arteriovenous malformations in matrix Gla protein deficiency. Proc Natl Acad Sci USA. 2013; 110(47):19071-19076.
18. Starosolski Z, Villamizar C A, Rendon D, et al. Ultra High-Resolution In vivo Computed Tomography Imaging of Mouse Cerebrovasculature Using a Long Circulating Blood Pool Contrast Agent. Scientific reports. 2015; 5:10178.
19. Lu L, Bischoff J, Mulliken J B, Bielenberg D R, Fishman S J, Greene A K. Progression of arteriovenous malformation: Possible role of vasculogenesis. Plast Reconstr Surg. 2011; 128(4):260e-269e.
20. Gillespie E, Leeman S E, Watts L A, et al. Plasminogen activator inhibitor-1 is increased in colonic epithelial cells from patients with colitis-associated cancer. J Crohns Colitis. 2013; 7(5):403-411.
21. Hjortland G O, Lillehammer T, Somme S, et al. Plasminogen activator inhibitor-1 increases the expression of VEGF in human glioma cells. Exp Cell Res. 2004; 294(1):130-139.
22. Zubac D P, Wentzel-Larsen T, Seidal T, Bostad L. Type 1 plasminogen activator inhibitor (PAI-1) in clear cell renal cell carcinoma (CCRCC) and its impact on angiogenesis, progression and patient survival after radical nephrectomy. BMC Urology. 2010; 10(1):20.
23. Volpert O V, Pili R, Sikder H A, et al. Id1 regulates angiogenesis through transcriptional repression of thrombospondin-1. Cancer Cell. 2002; 2(6):473-483.
24. Yao Y, Zebboudj A F, Shao E, Perez M, Boström K. Regulation of bone morphogenetic protein-4 by matrix GLA protein in vascular endothelial cells involves activin-like kinase receptor 1. J Biol Chem. 2006; 281(45):33921-33930.
25. Ng I, Tan W L, Ng P Y, Lim J. Hypoxia inducible factor-1alpha and expression of vascular endothelial growth factor and its receptors in cerebral arteriovenous malformations. J Clin Neurosci. 2005; 12(7):794-799.
26. Rothhammer T, Bataille F, Spruss T, Eissner G, Bosserhoff A K. Functional implication of BMP4 expression on angiogenesis in malignant melanoma. Oncogene. 2007; 26(28):4158-4170.
27. Rezzola S, Di Somma M, Corsini M, et al. VEGFR2 activation mediates the pro-angiogenic activity of BMP4. Angiogenesis. 2019; 22(4):521-533.
28. Takagi Y, Kikuta K, Moriwaki T, et al. Expression of thioredoxin-1 and hypoxia inducible factor-1alpha in cerebral arteriovenous malformations: Possible role of redox regulatory factor in neoangiogenic property. Surgical neurology international. 2011; 2:61.
29. Li L, Pan H, Wang H, et al. Interplay between VEGF and Nrf2 regulates angiogenesis due to intracranial venous hypertension. Scientific reports. 2016; 6(1):37338.
30. Lim C S, Kiriakidis S, Sandison A, Paleolog E M, Davies A H. Hypoxia-inducible factor pathway and diseases of the vascular wall. Journal of Vascular Surgery. 2013; 58(1):219-230.
31. Shi Y-H, Fang W-G. Hypoxia-inducible factor-1 in tumour angiogenesis. World J Gastroenterol. 2004; 10(8):1082-1087.
32. Zimna A, Kurpisz M. Hypoxia-Inducible Factor-1 in Physiological and Pathophysiological Angiogenesis: Applications and Therapies. Biomed Res Int. 2015; 2015:549412-549412.
33. Hashimoto T, Shibasaki F. Hypoxia-Inducible Factor as an Angiogenic Master Switch. Frontiers in Pediatrics. 2015; 3(33).
34. Montoya M M, Maul J, Singh P B, et al. A distinct inhibitory function for miR-18a in Th17 cell differentiation. J Immunol. 2017; 199(2):559-569.
35. Krutilina R, Sun W, Sethuraman A, et al. MicroRNA-18a inhibits hypoxia-inducible factor 1α activity and lung metastasis in basal breast cancers. Breast Cancer Research. 2014; 16(4):R78.
36. Wu F, Huang W, Wang X. microRNA-18a regulates gastric carcinoma cell apoptosis and invasion by suppressing hypoxia-inducible factor-1α expression. Exp Ther Med. 2015; 10(2):717-722.
37. Wang J, Greene S B, Bonilla-Claudio M, et al. Bmp signaling regulates myocardial differentiation from cardiac progenitors through a MicroRNA-mediated mechanism. Developmental cell. 2010; 19(6):903-912.
38. Li L, Shi J-Y, Zhu G-Q, Shi B. MiR-17-92 cluster regulates cell proliferation and collagen synthesis by targeting TGFB pathway in mouse palatal mesenchymal cells. Journal of Cellular Biochemistry. 2012; 113(4):1235-1244.
39. Luo T, Cui S, Bian C, Yu X. Crosstalk between TGF-β/Smad3 and BMP/BMPR2 signaling pathways via miR-17-92 cluster in carotid artery restenosis. Molecular and Cellular Biochemistry. 2014; 389(1):169-176.
40. Schwentner R, Herrero-Martin D, Kauer M O, et al. The role of miR-17-92 in the miRegulatory landscape of Ewing sarcoma. Oncotarget. 2017; 8(7):10980-10993.
41. Shao E S, Lin L, Yao Y, Bostrom K I. Expression of vascular endothelial growth factor is coordinately regulated by the activin-like kinase receptors 1 and 5 in endothelial cells. Blood. 2009; 114(10):2197-2206.
42. Yao Y, Shao E S, Jumabay M, Shahbazian A, Ji S, Bostrom K I. High-density lipoproteins affect endothelial BMP-signaling by modulating expression of the activin-like kinase receptor 1 and 2. Arteriosclerosis, thrombosis, and vascular biology. 2008; 28(12):2266-2274.
43. Yao Y, Shahbazian A, Bostrom K I. Proline and gamma-carboxylated glutamate residues in matrix Gla protein are critical for binding of bone morphogenetic protein-4. Circulation research. 2008; 102(9):1065-1074.
44. Beets K, Staring M W, Criem N, et al. BMP-SMAD signalling output is highly regionalized in cardiovascular and lymphatic endothelial networks. BMC Dev Biol. 2016; 16(1):34.
45. Dews M, Fox J L, Hultine S, et al. The myc-miR-17-92 axis blunts TGF{beta} signaling and production of multiple TGF{beta}-dependent antiangiogenic factors. Cancer Res. 2010; 70(20):8233-8246.
46. Han F, Wu Y, Jiang W. MicroRNA-18a Decreases Choroidal Endothelial Cell Proliferation and Migration by Inhibiting HIF1A Expression. Med Sci Monit. 2015; 21:1642-1647.
47. Kietzmann T, Samoylenko A, Roth U, Jungermann K. Hypoxia-inducible factor-1 and hypoxia response elements mediate the induction of plasminogen activator inhibitor-1 gene expression by insulin in primary rat hepatocytes. Blood. 2003; 101(3):907-914.
48. Kunz P, Sahr H, Lehner B, Fischer C, Seebach E, Fellenberg J. Elevated ratio of MMP2/MMP9 activity is associated with poor response to chemotherapy in osteosarcoma. BMC cancer. 2016; 16:223.
49. Xu M, Xu H, Qin Z, Zhang J, Yang X, Xu F. Increased expression of angiogenic factors in cultured human brain arteriovenous malformation endothelial cells. Cell Biochem Biophys. 2014; 70(1):443-447.
50. Hashimoto T, Wen G, Lawton M T, et al. Abnormal expression of matrix metalloproteinases and tissue inhibitors of metalloproteinases in brain arteriovenous malformations. Stroke. 2003; 34(4):925-931.
51. Schelter F, Kobuch J, Moss M L, et al. A disintegrin and metalloproteinase-10 (ADAM-10) mediates DN30 antibody-induced shedding of the Met surface receptor. J Biol Chem. 2010; 285(34):26335-26340.
52. Li D, Xiao, Z., Wang, G., & Song, X. Knockdown of ADAM10 inhibits migration and invasion of fibroblast-like synoviocytes in rheumatoid arthritis. Mol Med Rep. 2015; 12:5517-5523.
53. Marín-Ramos N I, Pérez-Hernández M, Anson T, et al. NEO100 targets glioma stem cells and inhibits motility through the calpain-1/RhoA pathway. J Neurosurgery. 2019:DOI: 10.3171/2019.3175.JNS19798.
54. Wang W, Swenson S, Cho H Y, Hofman F M, Schonthal A H, Chen T C. Efficient brain targeting and therapeutic intracranial activity of bortezomib through intranasal co-delivery with NEO100 in rodent glioblastoma models. J Neurosurg. 2019; doi: 10.3171/2018.11.JNS181161:1-9.
55. Nielsen C M, Huang L, Murphy P A, Lawton M T, Wang R A. Mouse models of cerebral arteriovenous malformation. Stroke. 2016; 47(1):293-300.
56. Gezercan Y, Acik V, Çavuş G, et al. Six different extremely calcified lesions of the brain: brain stones. Springerplus. 2016; 5(1):1941-1941.
57. Bokhari M R, Bokhari S R A. Brain, Arteriovenous Malformation. StatPearls Publishing, Treasure Island (FL); 2018.
58. Binder B R, Mihaly J. The plasminogen activator inhibitor “paradox” in cancer. Immunol Lett. 2008; 118(2):116-124.
59. Steingrimur S, Grainne A M, Eric P, Daniel A L. Plasminogen Activator Inhibitor-1 in tumor growth, angiogenesis and vascular remodeling. Curr Pharm Des. 2003; 9(19):1545-1564.
60. Zebboudj A F, Imura M, Bostrom K. Matrix GLA protein, a regulatory protein for bone morphogenetic protein-2. J Biol Chem. 2002; 277(6):4388-4394.
61. Bostrom K I, Jumabay M, Matveyenko A, Nicholas S B, Yao Y. Activation of vascular bone morphogenetic protein signaling in diabetes mellitus. Circulation research. 2011; 108(4):446-457.
62. Schliermann A, Nickel J. Unraveling the Connection between Fibroblast Growth Factor and Bone Morphogenetic Protein Signaling. International journal of molecular sciences. 2018; 19(10).
63. Kyono A, Avishai N, Ouyang Z, Landreth G E, Murakami S. FGF and ERK signaling coordinately regulate mineralization-related genes and play essential roles in osteocyte differentiation. J Bone Miner Metab. 2012; 30(1):19-30.
64. Neal A, Nornes S, Payne S, et al. Venous identity requires BMP signalling through ALK3. Nature Communications. 2019; 10(1):453.
65. Thomas J M, Surendran S, Abraham M, Rajavelu A, Kartha C C. Genetic and epigenetic mechanisms in the development of arteriovenous malformations in the brain. Clin Epigenetics. 2016; 8:78-78.
66. Mahmoud M, Allinson K R, Zhai Z, et al. Pathogenesis of arteriovenous malformations in the absence of endoglin. Circulation research. 2010; 106(8):1425-1433.
67. Sorensen L K, Brooke B S, Li D Y, Urness L D. Loss of distinct arterial and venous boundaries in mice lacking endoglin, a vascular-specific TGFbeta coreceptor. Developmental biology. 2003; 261(1):235-250.
68. Urness L D, Sorensen L K, Li D Y. Arteriovenous malformations in mice lacking activin receptor-like kinase-1. Nat Genet 2000; 26:328-331
69. Krebs L T, Starling C, Chervonsky A V, Gridley T. Notch1 activation in mice causes arteriovenous malformations phenocopied by ephrinB2 and EphB4 mutants. Genesis. 2010; 48(3):146-150.
70. Murphy P A, Kim T N, Huang L, et al. Constitutively active Notch4 receptor elicits brain arteriovenous malformations through enlargement of capillary-like vessels. Proc Natl Acad Sci USA. 2014; 111(50):18007-18012.
71. Carlson T R, Yan Y, Wu X, et al. Endothelial expression of constitutively active <em>Notch4</em> elicits reversible arteriovenous malformations in adult mice. Proceedings of the National Academy of Sciences of the United States of America. 2005; 102(28):9884-9889.
72. Murphy P A, Lam M T Y, Wu X, et al. Endothelial Notch4 signaling induces hallmarks of brain arteriovenous malformations in mice. Proceedings of the National Academy of Sciences of the United States of America. 2008; 105(31):10901-10906.
73. Valdimarsdottir G, Goumans M-J, Rosendahl A, et al. Stimulation of Id1 Expression by Bone Morphogenetic Protein Is Sufficient and Necessary for Bone Morphogenetic Protein& #x2013; Induced Activation of Endothelial Cells. Circulation. 2002; 106(17):2263-2270.
74. Chen X, Wu L, Li D, et al. Radiosensitizing effects of miR-18a-5p on lung cancer stem-like cells via downregulating both ATM and HIF-1α. Cancer Med. 2018; 7(8):3834-3847.
75. Suzuki Y, Montagne K, Nishihara A, Watabe T, Miyazono K. BMPs promote proliferation and migration of endothelial cells via stimulation of VEGF-A/VEGFR2 and Angiopoietin-1/Tie2 signalling. J Biochem. 2008; 143(2):199-206.
76. Garcia de Vinuesa A, Abdelilah-Seyfried S, Knaus P, Zwijsen A, Bailly S. BMP signaling in vascular biology and dysfunction. Cytokine Growth Factor Rev. 2016; 27:65-79.
77. Rodrigues M, Xin X, Jee K, et al. VEGF Secreted by Hypoxic Müller Cells Induces MMP-2 Expression and Activity in Endothelial Cells to Promote Retinal Neovascularization in Proliferative Diabetic Retinopathy. Diabetes. 2013; 62(11):3863-3873.
78. Liu C-T, Bi K-W, Huang C-C, et al. Davallia bilabiata exhibits anti-angiogenic effect with modified MMP-2/TIMP-2 secretion and inhibited VEGF ligand/receptors expression in vascular endothelial cells. Journal of Ethnopharmacology. 2017; 196:213-224.
79. Ghosh S, Basu M, Roy S S. ETS-1 protein regulates vascular endothelial growth factor-induced matrix metalloproteinase-9 and matrix metalloproteinase-13 expression in human ovarian carcinoma cell line SKOV-3. J Biol Chem. 2012; 287(18):15001-15015.
80. Belotti D, Calcagno C, Garofalo A, et al. Vascular Endothelial Growth Factor Stimulates Organ-Specific Host Matrix Metalloproteinase-9 Expression and Ovarian Cancer Invasion. Mol Cancer Res. 2008; 6(4):525-534.
81. Al-Olabi L, Polubothu S, Dowsett K, et al. Mosaic RAS/MAPK variants cause sporadic vascular malformations which respond to targeted therapy. The Journal of clinical investigation. 2018; 128(4):1496-1508.
82. Nikolaev S I, Vetiska S, Bonilla X, et al. Somatic Activating KRAS Mutations in Arteriovenous Malformations of the Brain. New England Journal of Medicine. 2018; 378(3):250-261.
83. Starke R M, McCarthy D, Komotar R J, Connolly E S. Somatic KRAS Mutation Found in Sporadic Arteriovenous Malformations. Neurosurgery. 2018; 83(1):E14-E15.
84. Tsang W P, Kwok T T. The miR-18a* microRNA functions as a potential tumor suppressor by targeting on K-Ras. Carcinogenesis. 2009; 30(6):953-959.
85. Takeshita S, Fumoto T, Naoe Y, Ikeda K. Age-related Marrow Adipogenesis Is Linked to Increased Expression of RANKL. J Biol Chem. 2014; 289(24):16699-16710.
86. Carmona R, Pritz J, Bydder M, et al. Fat composition changes in bone marrow during chemotherapy and radiation therapy. Int J Radiat Oncol Biol Phys. 2014; 90(1):155-163.
87. Vaka S R K, Sammeta S M, Day L B, Murthy S N. Delivery of nerve growth factor to brain via intranasal administration and enhancement of brain uptake. J Pharm Sci. 2009; 98(10):3640-3646.
88. Erdő F, Bors L A, Farkas D, Bajza Á, Gizurarson S. Evaluation of intranasal delivery route of drug administration for brain targeting. Brain Research Bulletin. 2018; 143:155-170.

TABLE 3
SEQUENCES

| Primer | Sequence | SEQ ID NO |
|---|---|---|
| ALK1-forward | 5′-AGGGCAAACCAGCCATTG-3′ | SEQ ID NO: 1 |
| ALK1-reverse | 5′-GGTTGCTCTTGACCAGCACAT-3′ | SEQ ID NO: 2 |
| ALK2-forward | 5′-GACGTGGAGTATGGCACTATCG-3′ | SEQ ID NO: 3 |
| ALK2-reverse | 5′-CACTCCAACAGTGTAATCTGGCG-3′ | SEQ ID NO: 4 |
| ALK5-forward | 5′-GACAACGTCAGGTTCTGGCTCA-3′ | SEQ ID NO: 5 |
| ALK5-reverse | 5′-CCGCCACTTTCCTCTCCAAACT-3′ | SEQ ID NO: 6 |
| B2M-forward | 5′-CCACTGAAAAAGATGAGTATGCCT-3′ | SEQ ID NO: 7 |
| B2M-reverse | 5′-CCAATCCAAATGCGGCATCTTCA-3′ | SEQ ID NO: 8 |
| BMP4-forward | 5′-AGCATGTCAGGATTAGCCGA-3′ | SEQ ID NO: 9 |
| BMP4-reverse | 5′-TGGAGATGGCACTCAGTTCA-3′ | SEQ ID NO: 10 |
| BMP9-forward | 5′-CATTGTGCGGAGCTTCAGCATG-3′ | SEQ ID NO: 11 |
| BMP9-reverse | 5′-CTGGTGATCTGCTCATGCCTAG-3′ | SEQ ID NO: 12 |
| ID1-forward | 5′-GCCGAGGCGGCATGCGTTC-3′ | SEQ ID NO: 13 |
| ID1-reverse | 5′-TCCCTCAGATCCGGCGAGGC-3′ | SEQ ID NO: 14 |
| HIF-1α-forward | 5′-TATGAGCCAGAAGAACTTTTAGGC-3′ | SEQ ID NO: 15 |
| HIF-1α-reverse | 5′-CACCTCTTTTGGCAAGCATCCTG-3′ | SEQ ID NO: 16 |
| MGP-forward | 5′-CCTCAGCAGAGATGGAGAGCTA-3′ | SEQ ID NO: 17 |
| MGP-reverse | 5′-ATGGCGTAGCGTTCGCAAAGTC-3′ | SEQ ID NO: 18 |
| MMP2-forward | 5′-CCATTTTGATGACGATGAGCTATG-3′ | SEQ ID NO: 19 |
| MMP2-reverse | 5′-GTTGTACTCCTTGCCATTGAACAA-3′ | SEQ ID NO: 20 |
| MMP9-forward | 5′-TTGACAGCGACAAGAAGTGG-3′ | SEQ ID NO: 21 |
| MMP9-reverse | 5′-GCCATTCACGTCGTCCTTAT-3′ | SEQ ID NO: 22 |
| PAI-1-forward | 5′-CTCATCAGCCACTGGAAAGGCA-3′ | SEQ ID NO: 23 |
| PAI-1-reverse | 5′-GACTCGTGAAGTCAGCCTGAAAC-3′ | SEQ ID NO: 24 |
| hsa-miR-18a | 5′-UAGGUGCAUCUAGUGCAGAUAG-3′ | SEQ ID NO: 25 |
| HIF1A | 5′-UCAUUUUAAAAAAUGCACCUUU-3′ | SEQ ID NO: 26 |
| MGP(+) Forward | 5′-GCCACAATTTCTGCATCCTGC-3′ | SEQ ID NO: 27 |
| MGP(+) Reverse | 5′-CGGGAAAGATGAGGAAGAAGGG-3′ | SEQ ID NO: 28 |
| MGP(-) Forward | 5′-TGCCTGAAGTAGCGGTTGTA-3′ | SEQ ID NO: 29 |
| MGP(-) Reverse | 5′-TGAATGAACTGCAGGACGAGG-3′ | SEQ ID NO: 30 |

What is claimed is:

1. A method of delivering a polynucleotide to a cell, the method comprising:
contacting the cell with the polynucleotide and perillyl alcohol (POH).

2. The method of claim 1, wherein the polynucleotide and POH are provided in one composition.

3. The method of claim 1, wherein the polynucleotide and POH are provided in separate compositions.

4. The method of claim 1, wherein the polynucleotide and POH are mixed prior to contacting the cell.

5. The method of claim 1, wherein the polynucleotide is a microRNA (miRNA).

6. The method of claim 5, wherein the miRNA is miR-18a.

7. The method of claim 1, wherein the polynucleotide is a short interfering RNA (siRNA) or a short-hairpin RNA (shRNA).

8. A method of delivering a polynucleotide to a cell, the method comprising:
contacting the cell with the polynucleotide, perillyl alcohol (POH) and an Argonaute protein or a variant thereof.

9. The method of claim 8, wherein the Argonaute protein is Argonaute-2 (Ago-2).

10. The method of claim 8, wherein the polynucleotide, POH and the Argonaute protein or a variant thereof are provided in one composition.

11. The method of claim 8, wherein the polynucleotide, POH and the Argonaute protein or a variant thereof are provided in two or three compositions.

12. The method of claim 8, wherein the polynucleotide, POH and the Argonaute protein or a variant thereof are mixed prior to contacting the cell.

13. The method of claim 8, wherein the polynucleotide is a microRNA (miRNA).

14. The method of claim 13, wherein the miRNA is miR-18a.

15. The method of claim 8, wherein the polynucleotide is a short interfering RNA (siRNA) or a short-hairpin RNA (shRNA).

16. A method of delivering a polynucleotide to a subject, the method comprising administering the polynucleotide, perillyl alcohol (POH), and optionally an Argonaute protein or a variant thereof to the subject.

17. The method of claim 16, wherein the Argonaute protein is Argonaute-2 (Ago-2).

18. The method of claim 16, wherein the polynucleotide is a microRNA (miRNA).

19. The method of claim 16, wherein the polynucleotide is a short interfering RNA (siRNA) or a short-hairpin RNA (shRNA).

20. The method of claim 19, wherein the miRNA is miR-18a.

21. The method of claim 16, wherein the polynucleotide, POH and optionally the Argonaute protein or a variant thereof are provided in one composition.

22. The method of claim 16, wherein the polynucleotide, POH and optionally the Argonaute protein or a variant thereof are provided in two or three compositions.

23. The method of claim 16, wherein the polynucleotide, POH and optionally the Argonaute protein or a variant thereof are mixed prior to administration to the subject.

24. A method of treating a condition in a subject, the method comprising of administering a polynucleotide, perillyl alcohol (POH), and optionally an Argonaute protein or a variant thereof to the subject.

25. The method of claim 24, wherein the polynucleotide is a microRNA (miRNA).

26. The method of claim 25, wherein the miRNA is miR-18a.

27. The method of claim 24, wherein the polynucleotide is a short interfering RNA (siRNA) or a short-hairpin RNA (shRNA).

28. The method of claim 24, wherein the Argonaute protein is Argonaute-2 (Ago-2).

29. The method of claim 24, wherein the polynucleotide, POH and optionally the Argonaute protein or a variant thereof are provided in one composition.

30. The method of claim 24, wherein the polynucleotide, POH and optionally the Argonaute protein or a variant thereof are provided in separate compositions.

31. The method of claim 24, wherein the polynucleotide, POH and optionally the Argonaute protein or a variant thereof are mixed prior to administration to the subject.

32. The method of claim 24, wherein the condition is a neurovascular disease.

33. The method of claim 24, wherein the condition is a stroke.

34. The method of claim 24, wherein the condition is a tumor.

35. The method of claim 24, wherein the condition is a brain tumor, glioma, glioblastoma, and/or glioblastoma multiforme (GBM).

36. The method of claim 24, wherein the condition is a spinal cord injury.

37. The method of claim 24, wherein the administration is intranasal, intratumoral, intracranial, intraventricular, intrathecal, epidural, intradural, intravascular, intravenous, intraarterial, intramuscular, subcutaneous, intraperitoneal, or oral.

38. The method of claim 24, wherein the administration is given once, twice, three or more times.

39. The method of claim 24, further comprising administering an anti-angiogenic drug to the subject.

40. The method of claim 24, further comprising administering a chemotherapeutic agent to the subject.

41. A kit comprising: (i) a polynucleotide; (ii) perillyl alcohol (POH); and
(iii) instructions for delivering the polynucleotide to a cell or a subject using POH.

42. The kit of claim 41, wherein the polynucleotide is a microRNA (miRNA).

43. The kit of claim 42, wherein the miRNA is miR-18a.

44. The kit of claim 41, wherein the polynucleotide is a short interfering RNA (siRNA) or a short-hairpin RNA (shRNA).

45. A kit comprising: (i) a polynucleotide; (ii) perillyl alcohol (POH); (iii) an Argonaute protein or a variant thereof; and (iv) instructions for delivering the polynucleotide to a cell or a subject using the POH and the Argonaute protein or a variant thereof.

46. The kit of claim 45, wherein the polynucleotide is a microRNA (miRNA).

47. The kit of claim 46, wherein the miRNA is miR-18a.

48. The kit of claim 45, wherein the polynucleotide is a short interfering RNA (siRNA) or a short-hairpin RNA (shRNA).

49. The kit of claim 45, wherein the Argonaute protein is Argonaute-2 (Ago-2).


SEQUENCE LISTING (30 sequences)

SEQ ID NO: 1 - DNA, 18 bp, Artificial sequence
  synthetic
agggcaaaccagccattg

SEQ ID NO: 2 - DNA, 21 bp, Artificial sequence
  synthetic
ggttgctcttgaccagcacat

SEQ ID NO: 3 - DNA, 22 bp, Artificial sequence
  synthetic
gacgtggagtatggcactatcg

SEQ ID NO: 4 - DNA, 23 bp, Artificial sequence
  synthetic
cactccaacagtgtaatctggcg

SEQ ID NO: 5 - DNA, 22 bp, Artificial sequence
  synthetic
gacaacgtcaggttctggctca

SEQ ID NO: 6 - DNA, 22 bp, Artificial sequence
  synthetic
ccgccactttcctctccaaact

SEQ ID NO: 7 - DNA, 24 bp, Artificial sequence
  synthetic
ccactgaaaaagatgagtatgcct

SEQ ID NO: 8 - DNA, 23 bp, Artificial sequence
  synthetic
ccaatccaaatgcggcatcttca

SEQ ID NO: 9 - DNA, 20 bp, Artificial sequence
  synthetic
agcatgtcaggattagccga

SEQ ID NO: 10 - DNA, 23 bp, Artificial sequence
  synthetic
ccaatccaaatgcggcatcttca

SEQ ID NO: 11 - DNA, 22 bp, Artificial sequence
  synthetic
cattgtgcggagcttcagcatg

SEQ ID NO: 12 - DNA, 22 bp, Artificial sequence
  synthetic
ctggtgatctgctcatgcctag

SEQ ID NO: 13 - DNA, 19 bp, Artificial sequence
  synthetic
gccgaggcggcatgcgttc

SEQ ID NO: 14 - DNA, 20 bp, Artificial sequence
  synthetic
tccctcagatccggcgaggc

SEQ ID NO: 15 - DNA, 24 bp, Artificial sequence
  synthetic
tatgagccagaagaacttttaggc

SEQ ID NO: 16 - DNA, 23 bp, Artificial sequence
  synthetic
cacctcttttggcaagcatcctg

SEQ ID NO: 17 - DNA, 22 bp, Artificial sequence
  synthetic
cctcagcagagatggagagcta

SEQ ID NO: 18 - DNA, 21 bp, Artificial sequence
  synthetic
tgaatgaactgcaggacgagg

SEQ ID NO: 19 - DNA, 24 bp, Artificial sequence
  synthetic
ccattttgatgacgatgagctatg

SEQ ID NO: 20 - DNA, 24 bp, Artificial sequence
  synthetic
gttgtactccttgccattgaacaa

SEQ ID NO: 21 - DNA, 20 bp, Artificial sequence
  synthetic
ttgacagcgacaagaagtgg

SEQ ID NO: 22 - DNA, 20 bp, Artificial sequence
  synthetic
gccattcacgtcgtccttat

SEQ ID NO: 23 - DNA, 22 bp, Artificial sequence
  synthetic
ctcatcagccactggaaaggca

SEQ ID NO: 24 - DNA, 23 bp, Artificial sequence
  synthetic
gactcgtgaagtcagcctgaaac

SEQ ID NO: 25 - RNA, 22 nt, Homo sapiens
uaggugcaucuagugcagauag

SEQ ID NO: 26 - RNA, 22 nt, Homo sapiens
ucauuuuaaaaaaugcaccuuu

SEQ ID NO: 27 - DNA, 21 bp, Artificial sequence
  synthetic
gccacaatttctgcatcctgc

SEQ ID NO: 28 - DNA, 22 bp, Artificial sequence
  synthetic
cgggaaagatgaggaagaaggg

SEQ ID NO: 29 - DNA, 20 bp, Artificial sequence
  synthetic
tgcctgaagtagcggttgta

SEQ ID NO: 30 - DNA, 21 bp, Artificial sequence
  synthetic
tgaatgaactgcaggacgagg